SEQUENCE LISTING (30 sequences)

SEQ ID NO: 1 - protein, 10 aa, Mus musculus
GFSLSTSGMG

SEQ ID NO: 2 - protein, 7 aa, Mus musculus
IWWDDDK

SEQ ID NO: 3 - protein, 11 aa, Mus musculus
ARTRRYFPFAY

SEQ ID NO: 4 - protein, 6 aa, Mus musculus
QNVGTN

SEQ ID NO: 5 - protein, 4 aa, Mus musculus
SAST

SEQ ID NO: 6 - protein, 9 aa, Mus musculus
QQYNTDPLT

SEQ ID NO: 7 - protein, 5 aa, Mus musculus
SYGMH

SEQ ID NO: 8 - protein, 17 aa, Mus musculus
VIWYEGSNKYYADSVKG

SEQ ID NO: 9 - protein, 15 aa, Mus musculus
GGSMVRGDYYYGMDV

SEQ ID NO: 10 - protein, 11 aa, Mus musculus
RASQGISSALA

SEQ ID NO: 11 - protein, 7 aa, Mus musculus
DASSLES

SEQ ID NO: 12 - protein, 9 aa, Mus musculus
QQFNSYPYT

SEQ ID NO: 13 - protein, 449 aa, synthetic construct
QVTLRESGPALVKPTQTLTLTCTFSGFSLSTSGMGVGWIRQPPGKALEWLAHIWWDDDKYYNPSLKSRLTISKDTSKNQVVLTMTNMDPVDTATYYCARTRRYFPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 234 aa, synthetic construct
MGWSCIILFLVATATGVHSEIVMTQSPATLSVSPGERATLSCKASQNVGTNVAWYQQKPGQAPRLLIYSASYRYSGIPARFSGSGSGTEFTLTISSLQSEDFAVYYCQQYNTDPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECS

SEQ ID NO: 15 - protein, 449 aa, synthetic construct
QVTLRESGPALVKPTQTLTLTCTFSGFSLSTSGMGVGWIRQPPGKALEWLAHIWWDDDKYYNPSLKSRLTISKDTSKNQVVLTMTNMDPVDTATYYCARTRRYFPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - DNA, 33 bp, synthetic construct
  source 1..33: mol_type = other DNA
gcacctgaactcctggccggaccgtcagtcttc

SEQ ID NO: 17 - DNA, 41 bp, synthetic construct
  source 1..41: mol_type = other DNA
gcacctgaactcctggccggaccggacgtcttcctcttccc

SEQ ID NO: 18 - DNA, 40 bp, synthetic construct
  source 1..40: mol_type = other DNA
caacaaagccctcccactgcccgaggagaaaaccatctcc

SEQ ID NO: 19 - DNA, 33 bp, synthetic construct
  source 1..33: mol_type = other DNA
cgggaggagcagtacgccagcacgtaccgtgtg

SEQ ID NO: 20 - DNA, 35 bp, synthetic construct
  source 1..35: mol_type = other DNA
ctgaactcctgggggacgactcagtcttcctcttc

SEQ ID NO: 21 - DNA, 42 bp, synthetic construct
  source 1..42: mol_type = other DNA
gtggtggacgtgagcgacgaagacggtgaggtcaagttcaac

SEQ ID NO: 22 - DNA, 1350 bp, synthetic construct
  source 1..1350: mol_type = other DNA
caggtgaccctgagggagagcggccccgccctggtgaagcccacccagaccctgaccctgacctgcaccttcagcggcttcagcctgagcaccagcggcatgggcgtgggctggatcaggcagccccccggcaaggccctggagtggctggcccacatctggtgggacgacgacaagtactacaaccccagcctgaagagcaggctgaccatcagcaaggacaccagcaagaaccaggtggtgctgaccatgaccaacatggaccccgtggacaccgccacctactactgcgccaggaccaggaggtacttccccttcgcctattgggggcaaggtacccttgtaactgtctcgagtgctagtacgaaagggccatcagtttttcctctcgctccgtcctccaagagcacttcaggcggtacagccgcgttgggctgtttggtgaaagattatttcccagagccggttactgtaagttggaactcaggggcactgacgtctggtgtccataccttccctgccgtattgcagagttctggcctgtatagcttgtcaagcgtggtcactgtaccttcttcaagcctgggtactcagacttacatctgtaatgtgaatcacaaaccaagcaacactaaagtcgataagaaggtcgagccgaagtcatgcgacaaaacccatacttgccccccctgcccggctcctgaactgcttgccggtcctagcgttttcctcttcccgcccaaaccgaaagacactctcatgattagtcggaccccggaagtaacttgtgtggtagttgacgtgagccatgaggaccccgaggtgaagttcaattggtacgtcgatggagtggaagttcataacgcaaagactaaacctagagaagagcagtacaattcaacttaccgcgtagtcagcgtccttacagttcttcatcaagactggctgaacggaaaggagtataagtgcaaggtcagtaataaggcgttgcccgcccctattgaaaagaccattagtaaagccaaagggcagccccgagaaccgcaagtctacactttgcccccgtcccgcgatgaattgaccaagaatcaagtcagtctcacctgtctggtgaaaggattttacccgtctgacatagctgtagagtgggaatctaacggacagccagagaataactataagactactcccccggttctcgattcagacgggtctttcttcctttacagcaaattgacggtggataagagtaggtggcagcagggcaacgtgttctcatgcagcgtgatgcatgaagctttgcataaccattatacccagaagtctctgtcattgagcccagggaaatga

SEQ ID NO: 23 - DNA, 1184 bp, synthetic construct
  source 1..1184: mol_type = other DNA
caggtgaccctgagggagagcggccccgccctggtgaagcccacccagaccctgaccctgacctgcaccttcagcggcttcagcctgagcaccagcggcatgggcgtgggctggatcaggcagccccccggcaaggccctggagtggctggcccacatctggtgggacgacgacaagtactacaaccccagcctgaagagcaggctgaccatcagcaaggacaccagcaagaaccaggtggtgctgaccatgaccaacatggaccccgtggacaccgccacctactactgcgccaggaccaggaggtacttccccttcgcctattgggggcaaggtacccttgtaactgtctcgagtgctagtacgaaagggccatcagtttttcctctcgctccgtcctccaagagcacttcaggcggtacagccgcgttgggctgtttggtgaaagattatttcccagagccggttactgtaagttggaactcaggggcactgacgtctggtgtccataccttccctgccgtattgcagagttctggcctgtatagcttgtcaagcgtggtcactgtaccttcttcaagcctgggtactcagacttacatctgtaatgtgaatcacaaaccaagcaacactaaagtcgataagaaggtcgagccgaagtcatgcgacaaaacccatacttgccccccctgcccggctcctgaactgcttgccggtcctagcgttttcctcttcccgcccaaaccgaaagacactctcatgattagtcggaccccggaagtaacttgtgtggtagttgacgtgagccatgaggaccccgaggtgaagttcaattggtacgtcgatggagtggaagttcataacgcaaagactaaacctagagaagagcagtacaattcaacttaccgcgtagtcagcgtccttacagttcttcatcaagactggctgaacggaaaggagtataagtgcaaggtcagtaataaggcgttgcccctgcctgaggaaaagaccattagtaaagccaaagggcagccccgagaaccgcaagtctacactttgcccccgtcccgcgatgaattgaccaagaatcaagtcagtctcacctgtctggtgaaaggattttacccgtctgacatagctgtagagtgggaatctaacggacagccagagaataactataagac

SEQ ID NO: 24 - DNA, 704 bp, synthetic construct
  source 1..704: mol_type = other DNA
atgggatggtcatgtatcatcctttttctagtagcaactgcaaccggtgtacattctgagatcgtgatgacccagagccccgccaccctgagcgtgagccctggagaaagggccaccctgagctgtaaggccagccagaacgtgggaaccaacgtggcctggtaccagcagaagcctggacaggcccccagactgctgatctacagcgccagctacaggtacagcggcatccccgccaggttctccggcagcggctccggaaccgagttcaccctgaccatctcctccctgcagagcgaggacttcgccgtgtactactgccagcagtacaacaccgaccccctgaccttcggcggcggcaccaaggtggagatcaagaggaccgtggccgcccccagcgtgttcatcttcccccccagcgacgagcagctgaagagcggcaccgccagcgtggtgtgcctgctgaacaacttctaccccagggaggccaaggtgcagtggaaggtggacaacgccctgcagagcggcaacagccaggagagcgtgaccgagcaggacagcaaggacagcacctacagcctgagcagcaccctgaccctgagcaaggccgactacgagaagcacaaggtgtacgcctgcgaggtgacccaccagggcctgagcagccccgtgaccaagagcttcaacaggggcgagtgcagctg

SEQ ID NO: 25 - protein, 8 aa, Mus musculus
GYTFTRYW

SEQ ID NO: 26 - protein, 8 aa, Mus musculus
IYPGDGDT

SEQ ID NO: 27 - protein, 14 aa, Mus musculus
ARNPLTTATAWFVY

SEQ ID NO: 28 - protein, 6 aa, Mus musculus
ENIYSN

SEQ ID NO: 29 - protein, 9 aa, Mus musculus
QHFWGPPWT

SEQ ID NO: 30 - DNA, 33 bp, synthetic construct
  source 1..33: mol_type = other DNA
caacaaagccctcccacgccccatcgagaaaac

(19) United States
(12) Patent Application Publication
(10) Pub. No.: US 2025/0179204 A1
(43) Pub. Date: Jun. 5, 2025

(54) ANTI GLUCOCORTICOID-INDUCED TNFR-RELATED (GITR) PROTEIN ANTIBODIES AND USES THEREOF

(71) Applicant: YEDA RESEARCH AND DEVELOPMENT CO. LTD., Rehovot (IL)

(72) Inventors: Rony DAHAN, Rehovot (IL); Yahel AVRAHAM, Rehovot (IL)

(73) Assignee: YEDA RESEARCH AND DEVELOPMENT CO. LTD., Rehovot (IL)

(21) Appl. No.: 18/843,848

(22) PCT Filed: Mar. 14, 2023
(86) PCT No.: PCT/IL2023/050267

§ 371 Date: Sep. 4, 2024

(30) Foreign Application Priority Data
Mar. 15, 2022 (IL) 291390

(51) Int. Cl. C07K 16/28; A61K 39/00; A61K 45/06; A61P 35/00; A61P 37/04

(52) U.S. Cl. CPC C07K 16/2878; A61K 45/06; A61P 35/00; A61P 37/04; A61K 2039/505; C07K 2317/24; C07K 2317/41; C07K 2317/52; C07K 2317/55; C07K 2317/565

(57) ABSTRACT

The invention provides agonistic and non-agonistic antibodies that specifically bind Glucocorticoid-induced TNFR-related (GITR) protein, comprising a modified human constant region (Fc) and optionally reduced fucosylation content. Also provided are polynucleotide sequences encoding the antibodies, pharmaceutical compositions thereof and methods of treating cancer.

### FIELD OF THE INVENTION

The present invention is in the field of immunotherapy and relates to engineered antibodies, to polynucleotide sequences encoding them, and to pharmaceutical compositions and therapeutic uses.

### BACKGROUND OF THE INVENTION

Cancer treatments have advanced significantly over the last decade, driven by immunomodulatory therapies that enhance host anti-tumor defense. In particular, immune checkpoint inhibitors, such as monoclonal antibodies (mAbs) that target the immune system rather than directly recognizing the tumor, have been shown to elicit durable responses in subsets of treated patients across multiple tumor types (Ribas, A. & Wolchok, J. D. Cancer immunotherapy using checkpoint blockade. Science 80-, 359, 1350-1355, 2018). These antibodies can be split into two groups, based on their means of inducing an anti-tumor immune response: (i) agonistic mAbs that stimulate activating co-regulatory receptors in the immune synapse (Mayes et al., A. The promise and challenges of immune agonist antibody development in cancer. Nature Reviews Drug Discovery, 17, 509-527, 2018), and (ii) antagonistic mAbs that block inhibitory signaling pathways in the immune synapse, and thereby dampen regulatory (suppressive) mechanisms (Sharma et al., The future of immune checkpoint therapy. Science 348, 56-61, 2015).

Antibodies consist of two structural regions: a variable fragment (Fab) that mediates antigen binding and a constant fragment (Fc) that mediates downstream effector functions via its interaction with Fc-receptors on (innate) immune cells or with proteins of the complement system.

Fcγ Receptors (FcγRs) belong to the immunoglobulin superfamily and share many structural and functional properties. FcγRs can be classified as activating or inhibitory, based on the presence of intracellular activating (immunoreceptor tyrosine activation motif, ITAM) or inhibitory (immunoreceptor tyrosine inhibition motif, ITIM)) signaling motifs. Three activating FcγRs are expressed in mice (FcγRI, FcγRIII, and FcγRIV) and humans (FcγRI, FcγRIIA, and FcγRIIIA), and a single inhibitory FcγRIIB, is expressed in both species. Each FcγR has a distinct pattern of cellular expression. Engagement of activating FcγRs induces effector mechanisms such as antibody-dependent cellular cytotoxicity (ADCC), phagocytosis, and production of pro-inflammatory cytokines. Signaling through the inhibitory receptor, FcγRIIB, results in negative functions such as inhibition of inflammatory immune responses. Engagement of activating or inhibitory FcγRs by Fc domains of antibodies is determined by the IgG subclass and Fc glycan composition (Pincetic, A. et al., Type I and type II Fc receptors regulate innate and adaptive immunity. Nat. Immunol. 15, 707-716, 2014).

Dendritic cells are a heterogenous population of antigen presenting cells that infiltrate tumors. While dendritic cells play a critical role in the priming and maintenance of local immunity, their functions are often diminished, or suppressed, by factors encountered in the tumor microenvironment. Furthermore, dendritic cell populations with immunosuppressive activities are also recruited to tumors, limiting T cell infiltration and promoting tumor growth. Activation of dendritic cells plays a critical role in priming anti-tumor T cell immunity and thereby represent a major therapeutic target for cancer immunotherapy (Whily et al., Dendritic Cells and Cancer: From Biology to Therapeutic Intervention. Cancer 11, 4, 521. 2019).

The main principle guiding the rationale design and development of antibodies so far has been the need to engineer their Fab domains to enhance their effect on specific receptor targets on immune cells. Recent in vivo findings, however, highlight the importance of the Fc domain for enhancing the potency of checkpoint antibody inhibitors by recruiting additional types of immune cells expressing either activating and/or inhibitory FcγRs (Dahan, R. et al., Therapeutic Activity of Agonistic, Human Anti-CD40 Monoclonal Antibodies Requires Selective FcγR Engagement. Cancer Cell 29, 820-831, 2016; Vargas, F. A. et al., Fc Effector Function Contributes to the Activity of Human Anti-CTLA-4 Antibodies. Cancer Cell 33, 649-663.e4, 2018).

Fc engagement of activating FcγRs is required to induce antibody effector functions, such as intratumoral regulatory T cells (Tregs) elimination by anti-CTLA4 (cytotoxic T-lymphocyte-associated protein 4/CD125), anti-OX-40 (CD134), or anti-4-1BB (Bulliard, Y. et al., OX40 engagement depletes intratumoral Tregs via activating FcγRs, leading to anti-tumor efficacy. Immunol. Cell Biol. 92, 475-480, 2014; Buchan, S. L. et al., Antibodies to Costimulatory Receptor 4-1BB Enhance Anti-tumor Immunity via T Regulatory Cell Depletion and Promotion of CD8 T Cell Effector Function. Immunity 49, 958-970.e7, 2018). Another Fc engagement can induce the reprograming of FcγR⁺ myeloid and natural killer cells (NK) in the tumor microenvironment (TME), for example by anti-PD-L1 (Programmed death ligand 1) and anti-CD73 mAbs. (Bulliard, Y. et al., Activating Fc 7 receptors contribute to the anti-tumor activities of immunoregulatory receptor-targeting antibodies. J. Exp. Med. 210, 1685-1693, 2013; Dahan, R. et al., FcγRs Modulate the Anti-tumor Activity of Antibodies Targeting the PD-1/PD-L1 Axis. Cancer Cell 28, 285-295, 2015; and Dahan, R. & Ravetch, J. V. Co-targeting of Adenosine Signaling Pathways for Immunotherapy: Potentiation by Fc Receptor Engagement. Cancer Cell 30, 2016).

Conversely, engagement of the inhibitory FcγRIIB provides an inert scaffold for antibody cross-linking to enhance agonistic mAb activities mainly those targeting tumor necrosis factor receptors (TNFRs), such as anti-CD40 mAbs (Li, F. & Ravetch, J. V. Anti-tumor activities of agonistic anti-TNFR antibodies require differential FcRγIIB co-engagement in vivo. Proc. Natl. Acad. Sci., 110(48), 19501-6, 2013). Thus, agonistic mAbs can work through multiple mechanisms in vivo (e.g., TNFR activation and Treg depletion), which can synergize their activities for optimal therapeutic efficacy. Hence, the selection of the appropriate scaffold (comprised of an IgG isoform or Fc region variant that selectively engages or avoids specific FcγR pathways) for each antibody is extremely important for achieving optimal anti-tumor activity.

One of the aforementioned immune checkpoints molecules is the Glucocorticoid-induced TNFR related (GITR, also termed TNFRSF18 and CD357) protein. GITR is a member of the cell surface TNF receptor superfamily, constitutively expressed at high levels on regulatory T cells (Tregs) and at low levels on Naïve and memory T cells (G. Nocentini, C. Riccardi GITR: a modulator of immune response and inflammation. Adv Exp Med Biol, 647 156-173, 2009).

GITR triggering co-activates effector T lymphocytes and modulates regulatory T cell (Treg) activity. GITR is activated by GITR ligand (GITRL), which is mainly expressed on antigen presenting cells (APC). GITR activation increases resistance to tumors and viral infections, is involved in autoimmune/inflammatory processes and regulates leukocyte extravasation. GITR is thus an attractive target for immunotherapy, owing to its capacity to promote effector T cell functions and hamper regulatory T cell suppression.

Activation of T cells by a number of different stimuli rapidly increases GITR expression within 24 hours, on both regulatory T cells (Treg) and effector T cells (Teff), (L. T. Krausz, et al., GITR-GITRL system, a novel player in shock and inflammation. Scientific World Journal, 7 533-566, 2007). FoxP3, a key regulator protein of Tregs development and function, promotes high-level GITR expression in mature T cells, while in activated T cells, canonical NFκB signaling induces GITR expression, suggesting a cell type-intrinsic regulation of expression (Y. Zhan, et al., Glucocorticoid-induced TNF receptor expression by T cells is reciprocally regulated by NF-kappaB and NFAT. J Immunol, 181 5405-5413, 2008).

Low to moderate levels of GITR are also detected on innate immune cells following activation (S. Hanabuchi, et al., Human plasmacytoid predendritic cells activate NK cells through glucocorticoid-induced tumor necrosis factor receptor-ligand (GITRL). Blood, 107 3617-3623, 2006). Within innate cell types, the highest induction is observed on activated natural killer cells (NK) with levels comparable to GITR expression on activated T effectors (Teff) cells. Only intermediate levels are seen on activated macrophage and dendritic cells (DCs) (D. L. Clouthier, T. H. Watts Cell-specific and context-dependent effects of GITR in cancer, autoimmunity, and infection. Cytokine Growth Factor Rev, 25 91-106, 2014).

High level of GITR expression in activated Tregs is an important distinction that becomes more apparent during the in vivo evaluation of GITR modulation. Numerous studies report, that tumor infiltrating Treg expressing GITR cells are present in tumors such as Non-Small Cell Lung Carcinoma (NSCLC), renal cell carcinoma, melanoma, glioblastoma, colorectal cancer, breast cancer and ovarian carcinoma, (Curiel, T., et al., Specific recruitment of regulatory T cells in ovarian carcinoma fosters immune privilege and predicts reduced survival. Nat Med 10, 942-949, 2004, Zhu et al., Evaluation of glucocorticoid-induced TNF receptor (GITR) expression in breast cancer and across multiple tumor types. Mod Pathol 33, 1753-1763, 2020; Vence L, et al. Characterization and Comparison of GITR Expression in Solid Tumors. Clin Cancer Res. 2019, 25(21), 6501-6510).

Anti mouse GITR antibodies were found to depend on FcγR-mediated Treg depletion and CD8 T cells proliferation for anti-tumor activity (D. Coe, et al., Depletion of regulatory T cells by anti-GITR mAb as a novel mechanism for cancer immunotherapy. Cancer Immunol Immunotherapy, 59, 1367-1377, 2010).

Anti-GITR agonist antibodies, as a monotherapy or in combination with additional immunotherapies, demonstrate significant therapeutic potential by enhancing effector T cells and inhibiting Tregs responses in the tumor microenvironment (TME), as demonstrated in pre-clinical models (A. D. Cohen et al., Agonist anti-GITR monoclonal antibody induces melanoma tumor immunity in mice by altering regulatory T cell stability and intra-tumor accumulation. PLoS One. 5, 2010; D. A. Schaer, J. et al., Modulation of GITR for cancer immunotherapy. Curr. Opin. Immunol. 24 217-224 2012; L. Lu, et al. Combined PD-1 blockade and GITR triggering induce a potent anti-tumor immunity in murine cancer models and synergizes with chemotherapeutic drugs. J Transl Med, 12, 36-47, 2014; Zappasodi R, et al. Rational design of anti-GITR-based combination immunotherapy. Nat Med. 2019, 25(5), 759-766). However, significant barriers were encountered to its clinical translation: the promising preclinical activity is yet to be recapitulated in clinical trials (Tran B., et al., Dose escalation results from a first-in-human, phase 1 study of glucocorticoid-induced TNF receptor-related protein agonist AMG 228 in patients with advanced solid tumors. J Immunother Cancer. 2018, 6(1), 93; and Siu L L, et al., Preliminary results of a phase I/II a study of BMS-986156, glucocorticoid-induced tumor necrosis factor receptor-related gene (GITR) agonist), alone and in combination with nivolumab in patients with advanced solid tumors. J Clin Oncol. 2017, 35(15_suppl), 104).

The anti-mouseGITR (mGITR) agonist antibody termed DTA-1 is an IgG2b rat antibody most widely used in research (J. Shimizu, et al., Stimulation of CD25+CD4+ regulatory T cells through GITR breaks immunological self-tolerance. Nat Immunol. 3, 135-142, 2002). Currently, there are few antibodies that are in pre-clinical, or clinical (phase I/II) trials, such as MK-4166 of Merck & Co Inc. (U.S. Pat. No. 8,709,424), ragifilimab of Incyte Corp. (U.S. Ser. No. 10/155,818), REGN-6569 of Regeneron Pharmaceuticals Inc. (U.S. Ser. No. 10/738,126), PTZ-522 of Potenza Therapeutics Inc. (US2018208665), GWN-323 of Novartis AG (U.S. Ser. No. 10/662,247), and BMS-986156 of Bristol-Myers Squibb Co. (U.S. Pat. No. 9,228,016). WO2015187835 discloses anti human GITR (hGITR) antibodies having engineered Fc region to have increased binding to FcγRIIB and increased ADCC activity.

U.S. Pat. Nos. 7,812,135, 1,121,358 and US2019010241 disclose specific mAbs to human GITR.

U.S. Pat. No. 9,040,041 discloses a method of enhancing activation of antibody dependent cellular phagocytosis (ADCP) by an antibody that comprises engineered human Fc. Protein targets for the engineered antibodies are Ep-CAM, CD19, CD20, CD22, CD30, CD33, CD40, CD40L, CD52, Her2/neu, EGFR, IGF-1R, EpCAM, MUC1, GD3, CEA, CA 125, HLA-DR, MUC18, and prostate specific membrane antigen (PMSA).

WO2019/125846 discloses human IgG Fc region variants specific to the tumor antigen sLeA, with improved effector function, having higher binding affinity to hFcγRIIA, hFcγRIIIA, and hFcγRIIB. One of the antibodies, hIgG1-G236A/A330I/I332E (GAALIE) has enhanced binding affinity to activatory hFcRIIA and hFcRIIIA and lower binding for the inhibitory FcγRIIB and also exhibits enhanced ADCC activity.

Weitzenfeld P., et al., disclose carbohydrate-targeting antibodies comprising combinations of specific mutations in the Fc domain, that result in enhancing the affinity for both activating αyRs, hFcγRIIA and hFcγRIIIA, while reducing the binding to the inhibitory receptor, hFcγRIIB (Antibodies targeting sialyl Lewis A mediate tumor clearance through distinct effector pathways. J. Clin. Invest, 129, 3952-3962, 2019).

U.S. patent Ser. No. 10/479,838 discloses specific antibodies that bind CD40 comprising a Fc region modified to enhance specificity of binding to FcγRIIB.

WO2003035835 discloses a cell line with reduced ability to attach fucose to N297-linked carbohydrates, resulting in hypofucosylation of the expressed antibodies, wherein about 80-100% of the glycoprotein comprises a mature core carbohydrate structure which lacks fucose. The antibodies produced bind human FcγRIIIA and have improved ADCC activity relative to fucosylated antibodies. One of the antibody variants disclosed has an increased binding to FcγRIIA and to FcγRIIIB.

The outcome of human FcγR interactions with anti-human GITR antibodies has not been comprehensively addressed and there is still an unmet need for appropriate IgG scaffolds to improve the anti-tumor activity of therapeutic anti GITR antibodies through selective binding to Fcγ receptors in the TME.

### SUMMARY OF THE INVENTION

The present invention provides, according to some embodiments, antibodies that bind Glucocorticoid-induced TNFR-related (GITR), having improved therapeutic anti-tumor activity compared to previously known antibodies to this protein. The present invention provides anti GITR antibodies comprising Fc modifications that result in increased binding to at least one of activating receptors FcγRIIA and FcγRIIIA, and optionally decreased binding to the inhibitory FcγRIIB receptor. It was unexpectedly found that the Fc-modified antibodies of the present invention, although having high binding ratio to FcγRIIA and/or FcγRIIIA relative to FcγRIIB and thus unable to engages FcγRIIB-mediated antibody cross-linking, elicit enhanced in vivo anti-tumor effect and long-term anti-tumor immunity. Antibodies having the same Fab region and lower binding ratio to FcγRIIA and/or FcγRIIIA relative to FcγRIIB, failed to elicit anti-tumor effect and long-term anti-tumor immunity.

The present invention provides therapeutic antibodies that bind with high affinity and specificity through their Fab region to GITR, and through their Fc region to activating FcγRIIA and FcγRIIIA receptors, with comparable lower binding affinity to the inhibitory receptor FcγRIIB. The antibodies of the present invention are characterized by having a modified human IgG1 Fc region wherein the modified human IgG1 Fc region comprises a substitution of the Glycine residue at position 236 with an Alanine residue (G236A), and at least one further modification selected from: reduction in the glycosylation content (e.g., afucosylation), and substitutions of Alanine at position 330 with Leucine (A330L), and of Isoleucine at position 332 with Glutamic acid (I332E). The numbering of the amino acid residues in the Fc region are according to the EU index.

The present invention thus provides, according to one aspect, an antibody, or a composition comprising at least one antibody, that specifically binds GITR, wherein the antibody comprises a variable region (Fab), and a modified human IgG1 constant region (Fc), wherein said antibody has increased binding ratio to (RIIA and/or RIIIA)/RIIB, compared to antibody having the same Fab and a parent non-modified human IgG1 Fc.

According to some embodiments, the human IgG1 Fc region modifications result in increase in the binding ratio of the antibody to RIIA/RIIB; RIIIA/RIIB; and (RIIA+RIIIA)/RIIB.

According to some embodiments of this aspect, the invention provides an antibody that specifically binds GITR, wherein the antibody comprises a variable region (Fab), and a modified human IgG1 Fc region, wherein the modified human IgG1 Fc region is selected from:
i. a human IgG1 Fc region comprising an Asparagine residue (N) at position 297, the substitution G236A of amino acid residue Glycine (G) at position 236 to Alanine residue (A), and a fucosylation content of up to about 40% out of all glycan structures (herein afuco-G236A); and
ii. a human IgG1 Fc region comprising the amino acid substitutions of: the Glycine residue at position 236 to an Alanine residue (G236A), substitution of the Alanine residue at position 330 to a Leucine residue (A330L), and substitution of the Isoleucine residue at position 332 to a Glutamic acid residue (I332E), (herein GAALIE).

The invention thus provides, according to some embodiments, an isolated afucosylated antibody that specifically binds Glucocorticoid-induced TNFR-related (GITR) protein, wherein the antibody comprises a variable region (Fab), and a modified human IgG1 constant region (Fc) comprising an Asparagine residue (N) at position 297, the substitution G236A of amino acid residue Glycine (G) at position 236 to Alanine residue (A), (herein afuco-G236A).

The invention also provides, according to yet other embodiments, an antibody that specifically binds Glucocorticoid-induced TNFR-related (GITR) protein, wherein the antibody comprises a variable region (Fab), and a modified human IgG1 constant region (Fc), wherein the modified human IgG comprises the amino acid substitutions of: the Glycine residue at position 236 to an Alanine residue (G236A), substitution of the Alanine residue at position 330 to a Leucine residue (A330L), and substitution of the Isoleucine residue at position 332 to a Glutamic acid residue (I332E), (herein GAALIE).

According to some embodiments, the GITR is human GITR (hGITR).

According to some embodiments, the antibody is a humanized antibody.

According to some embodiments, the antibody is a chimeric antibody.

Any binding site, hypervariable region (HVR) or Fab, of an antibody that specifically recognizes GITR, and particularly hGITR, can be used to generate the IgG1-Fc region modified antibodies of the present invention. This includes agonists, non-agonists, and mixed agonists/non-agonist antibodies, as well as antibodies that do not elicit response following binding to hGITR or do not mediate any of the activities associated with binding to hGITR. In some embodiments, binding to hGITR on T cells, is only used for targeting the antibodies of the invention to the required location of the immunological synapse while the modified Fc region mediates their activity on FcγR expressing cells.

According to some embodiments, the Fab of the anti hGITR antibody is derived from an agonist antibody that elicit GITR activation mediated by MAPK signaling.

According to some embodiments, the Fab of the anti hGITR antibody is derived from a non-agonist antibody.

According to some embodiments, the Fab of the anti-hGITR antibody comprises a set of 6 CDR sequences of an antibody specific to human GITR.

According to some embodiments, the antibody comprises a set of 6 CDR sequences, wherein the set is selected from the group consisting of:
i. Heavy chain (HC) CDR1 comprising the sequence GFSLSTSGMG (SEQ ID NO: 1), HC CDR2 comprising the sequence IWWDDDK (SEQ ID NO: 2). HC CDR3 comprising the sequence ARTRRYFPFAY (SEQ ID NO: 3), light chain (LC) CDR1 comprising the sequence QNVGTN (SEQ ID NO: 4). LC CDR2 comprising the sequence SAS or the sequence SAST (SEQ ID NO: 5), LC CDR3 comprising the sequence QQYNTDPLT (SEQ ID NO: 6); and
ii. HC CDR1 comprising the sequence SYGMH (SEQ ID NO: 7). HC CDR2 comprising the sequence VIWYEGSNKYYADSVKG (SEQ ID NO: 8), HC CDR3 comprising the sequence GGSMVRGDYYYGMDV (SEQ ID NO: 9), LC CDR1 comprising the sequence RASQGISSALA (SEQ ID NO: 10). LC CDR2 comprising the sequence DASSLES (SEQ ID NO: 11), and LC CDR3 comprising the sequence QQFNSYPYT (SEQ ID NO: 12).
iii. HC CDR1 comprising the sequence GYTFTRYW (SEQ ID NO: 25), HC CDR2 comprising the sequence IYPGDGDT (SEQ ID NO: 26). HC CDR3 comprising the sequence ARNPLTTATAWFVY (SEQ ID NO: 27). LC CDR1 comprising the sequence ENIYSN (SEQ ID NO: 28), LC CDR2 comprising the sequence AAT, and LC CDR3 comprising the sequence QHFWGPPWT (SEQ ID NO: 29).

Each option represents a separate embodiment of the present invention.

According to some embodiments, the afuco-G236A antibody comprises a human IgG1 heavy chain constant region comprising the sequences:

ASTKGPSVFPLAPSSKSTSGGTAALGCLVKDYFPEPVTVSWNS
GALTSGVHTFPAVLQSSGLYSLSSVVTVPSSSLGTQTYICNVN
HKPSNTKVDKKVEPKSCDKTHTCPPCPAPELLAGPSVFLFPPK
PKDTLMISRTPEVTCVVVDVSHEDPEVKFNWYVDGVEVHNAKT
KPREEQYNSTYRVVSVLTVLHQDWLNGKEYKCKVSNKALPAPI
EKTISKAKGQPREPQVYTLPPSRDELTKNQVSLTCLVKGFYPS
DIAVEWESNGQPENNYKTTPPVLDSDGSFFLYSKLTVDKSRWQ
QGNVFSCSVMHEALHNHYTQKSLSLSPGK
(SEQ ID NO: 13, wherein the G236A
substitution is underlined).

According to some embodiments, the afucosylated-G236A antibody comprises a human kappa light chain constant region comprising the sequence:

(SEQ ID NO: 14)
RTVAAPSVFIFPPSDEQLKSGTASVVCLLNNFYP
REAKVQWKVDNALQSGNSQESVTEQDSKDSTYSL
SSTLTLSKADYEKHKVYACEVTHQGLSSPVTKSF
NRGEC.

According to some embodiments, the afuco-G236A antibody comprises a heavy chain comprising the sequence set forth in SEQ ID NO: 13, and a light chain comprising a sequence set forth in SEQ ID NO: 14.

According to some embodiments, the GAALIE antibody (Fc region substitutions G236A, A330L, and 1332E) comprises the heavy chain constant region sequence:

ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKKVEPKSCDKTHTCPPCPAPELLAGPSVFLFPPKPKDTLM
ISRTPEVTCVVVDVSHEDPEVKFNWYVDGVEVHNAKTKPREEQYN
STYRVVSVLTVLHQDWLNGKEYKCKVSNKALPLPEEKTISKAKGQ
PREPQVYTLPPSRDELTKNQVSLTCLVKGFYPSDIAVEWESNGQP
ENNYKTTPPVLDSDGSFFLYSKLTVDKSRWQQGNVFSCSVMHEAL
HNHYTQKSLSLSPGK
(SEQ ID NO: 15, wherein the substitutions
are underlined).

According to some embodiments, the GAALIE antibody comprises a heavy chain comprising the sequence set forth in SEQ ID NO: 15, and a light chain comprising a sequence set forth in SEQ ID NO: 14.

According to some embodiments, the Fc region modification results in enhanced activation of dendritic cells (DCs), production of inflammatory cytokines or both.

According to some embodiments, the Fc region modification does not result in enhancement of ADCC activity.

According to some embodiments, the Fc region modification results in activation of dendritic cells leading to upregulation of DC activation markers, with no enhancement of ADCC.

Conjugates comprising the anti-hGITR antibodies described above are also within the scope of the present invention. These conjugates may include a moiety such as a detectable probe or a toxin attached to an anti GITR antibody of the present invention. The moiety may be attached to any part of the antibody as long as it does not interfere with its binding to hGITR and with binding to human FcγRIIA and FcγRIIIA.

The present invention also provides, according to another aspect, polynucleotide sequences encoding at least one chain of an antibody described above that specifically binds hGITR.

According to some embodiments, the antibody encoded by the polynucleotide sequences of the present invention, comprises a variable region (Fab) that recognizes hGITR, and a modified human IgG1 constant region (Fc), wherein the modified human IgG1 Fc region is selected from:
i. a human IgG1 Fc region comprising N at position 279 and the substitution G236A; and
ii. a human IgG1 Fc region comprising the amino acid substitutions: G236A, A330L, and 1332E (herein GAALIE).

According to some embodiments, the polynucleotide sequence encodes at least one chain of an antibody having high affinity and specificity for hGITR, wherein the antibody comprises a modified IgG1 Fc region comprising the substitution of amino acid residue Glycine at position 236 to Alanine residue (G236A), a substitution of amino acid residue Alanine at position 330 to Leucine residue (A330L), and a substitution of amino acid residue Isoleucine at position 332 to Glutamic acid residue (I332E).

According to some embodiments, the polynucleotide sequence encodes an amino acid heavy or light chain of an antibody described above.

According to some embodiments, the encoded heavy chain is human IgG1.

According to some embodiments, the polynucleotide sequence encodes at least one amino acid chain of an antibody selected a humanized antibody and a chimeric antibody.

According to some embodiments, the polynucleotide sequence encodes an antibody chain comprising an amino acid sequence set forth in any one of SEQ ID NOS: 13, 14 or 15, or an analog or derivative thereof having at least 90% sequence identity with any of said amino acid sequences. Each possibility represents a separate embodiment of the present invention.

According to some embodiments, the polynucleotide sequence encodes an antibody heavy chain amino acid sequence comprising a sequence selected from SEQ ID NO: 13 and SEQ ID NO: 15, or a variant thereof having at least 90% sequence identity with said amino acid sequence.

According to some embodiments, the polynucleotide that encodes an antibody heavy chain is set forth in a sequence selected from (SEQ ID NO: 22); and (SEQ ID NO: 23); or a variant thereof having at least 80% sequence identity with said polynucleotide sequence.

According to some embodiments, the polynucleotide sequence encodes an antibody light chain amino acid sequence comprising a sequence set forth in SEQ ID NO: 14, or a variant thereof having at least 90% sequence identity with said amino acid sequence.

According to some embodiments, the polynucleotide sequence that encodes an antibody light chain amino acid sequence comprises the sequence set forth in SEQ ID NO: 24, or a variant thereof having at least 80% sequence identity with said amino acid sequence.

Vectors, plasmids and constructs carrying the polynucleotide sequences disclosed above, are provided according to yet another aspect of the present invention.

According to some embodiments, the vector, plasmid or construct comprises a polynucleotide sequence encoding a heavy chain, wherein the polynucleotide sequence is selected from the group consisting of SEQ ID NO: 13 and SEQ ID NO: 15 or a variant thereof having at least 80% sequence identity with said polynucleotide sequence.

According to some embodiments, the vector, plasmid or construct comprises a polynucleotide sequence encoding a heavy chain selected from the group consisting of SEQ ID NO: 13 and 15 or a variant thereof having at least 80% sequence identity; and a polynucleotide sequence encoding a light chain of SEQ ID NO: 14 or a variant thereof having at least 80% sequence identity.

In still another aspect, the present invention provides a host cell or a population or a culture of host cells, comprising at least one of the above polynucleotide sequences, vectors or constructs, wherein these cells are capable of producing at least one chain of an antibody described above.

The present invention also provides, according to another aspect, a composition comprising a plurality of antibody molecules that specifically bind Glucocorticoid-induced TNFR-related (GITR) protein, wherein each antibody molecule comprises a variable region (Fab), and a modified human IgG1 constant region (Fc region), wherein the Fc region comprises an Asparagine residue (N) at position 297, the substitution G236A of amino acid residue Glycine (G) at position 236 to Alanine residue (A), and wherein about 65-100% of the antibody molecules in the composition comprise a mature core carbohydrate structure which lacks fucose, attached to the Asparagine residue (N) at position 297 of the Fc region.

According to some embodiments, about 80-100% of the antibody molecules in the composition comprise a mature core carbohydrate structure which lacks fucose, attached to the Asparagine residue (N) at position 297 of the Fc region.

According to yet other embodiments, about 82%, 83%, 84%, 85%, 86%, 87%, 88%, 89%, 80%, 91%, 92%, 93%, 94%, 95%, 96%, 97%, 98% or 99% of the antibody molecules in the composition comprise a mature core carbohydrate structure which lacks fucose, attached to the Asparagine residue (N) at position 297 of the Fc region.

According to other embodiments, at least 70%, 75% or 80% of the IgG Fc regions of the antibody molecules are non-fucosylated. According to some specific embodiments, about 60-80% of the antibody molecules are afucosylated. Each option represents a separate embodiment of the present invention.

According to yet other embodiments, the invention provides a composition comprising a plurality of afuco-G236A antibodies comprising less than 20%, less than 25%, or less than 30% fucosylation of their human IgG1 Fc regions. Each option represents a separate embodiment of the present invention.

According to some embodiments, the composition contains at least 50%, 60%, 70% 80% or 90% less fucose moieties attached to the N-297 of the human IgG1 Fc region of the afuco-G236A antibody molecules, compared to non-afucosylated antibodies. Each option represents a separate embodiment of the present invention.

According to some embodiments, the GITR is human GITR (hGITR).

According to some embodiments, the Fab of each of the antibody molecules comprises a set of 6 CDR sequences, wherein the set is selected from the group consisting of:
i. Heavy chain (HC) CDR1 comprising the sequence GFSLSTSGMG (SEQ ID NO: 1), HC CDR2 comprising the sequence IWWDDDK (SEQ ID NO: 2), HC CDR3 comprising the sequence ARTRRYFPFAY (SEQ ID NO: 3), light chain (LC) CDR1 comprising the sequence QNVGTN (SEQ ID NO: 4), LC CDR2 comprising the sequence SAS or the sequence SAST (SEQ ID NO: 5), LC CDR3 comprising the sequence QQYNTDPLT (SEQ ID NO: 6);
ii. HC CDR1 comprising the sequence SYGMH (SEQ ID NO: 7), HC CDR2 comprising the sequence VIWYEGSNKYYADSVKG (SEQ ID NO: 8). HC CDR3 comprising the sequence GGSMVRGDYYYGMDV (SEQ ID NO: 9), LC CDR1 comprising the sequence RASQGISSALA (SEQ ID NO: 10), LC CDR2 comprising the sequence DASSLES (SEQ ID NO: I1), and LC CDR3 comprising the sequence QQFNSYPYT (SEQ ID NO: 12); and
iii. HC CDR1 comprising the sequence GYTFTRYW (SEQ ID NO: 25). HC CDR2 comprising the sequence IYPGDGDT (SEQ ID NO: 26). HC CDR3 comprising the sequence ARNPLTTATAWFVY (SEQ ID NO: 27), LC CDR1 comprising the sequence ENIYSN (SEQ ID NO: 28). LC CDR2 comprising the sequence AAT, and LC CDR3 comprising the sequence QHFWGPPWT (SEQ ID NO: 29).

According to some embodiments, each of the antibody molecules comprises a heavy chain sequence set forth in SEQ ID NO: 13, a light chain sequence set forth in SEQ ID NO: 14, or both.

According to some embodiments, the antibody molecules are chimeric antibodies or humanized antibodies.

According to some embodiments, the composition is in the form of a pharmaceutical composition further comprising at least one carrier, excipient, or diluent.

Pharmaceutical compositions comprising at least one antibody or antibody conjugate described above that specifically binds GITR, and at least one carrier, excipient or diluent, are provided according to yet another aspect of the present invention.

According to some embodiments, the pharmaceutical composition comprises, as an active ingredient, an antibody comprising a variable region (Fab) that binds GITR, and a modified human IgG1 constant region (Fc), wherein the modified human IgG1 Fc region is selected from:
i. a human IgG1 Fc region comprising the substitution G236A and a fucosylation content of 20-40% out of all glycan structures (herein afuco-G236A); and
ii. a human IgG1 Fc region comprising the amino acid substitutions: G236A, A330L, and I332E (herein GAALIE).

According to some embodiments, the pharmaceutical composition comprises, as an active ingredient, an antibody or a plurality of antibodies selected from: an isolated afucosylated antibody that specifically binds Glucocorticoid-induced TNFR-related (GITR) protein, wherein the antibody comprises a variable region (Fab), and a modified human IgG1 constant region (Fc) comprising an Asparagine residue (N) at position 297, the substitution G236A of amino acid residue Glycine (G) at position 236 to Alanine residue (A), (herein afuco-G236A); and an antibody that specifically binds Glucocorticoid-induced TNFR-related (GITR) protein, wherein the antibody comprises a variable region (Fab), and a modified human IgG1 constant region (Fc), wherein the modified human IgG comprises the amino acid substitutions of: the Glycine residue at position 236 to an Alanine residue (G236A), substitution of the Alanine residue at position 330 to a Leucine residue (A330L), and substitution of the Isoleucine residue at position 332 to a Glutamic acid residue (I332E), (herein GAALIE).

According to some embodiments, the pharmaceutical composition comprises as an active ingredient, at least one antibody that specifically binds GITR, or a conjugate thereof, said antibody comprises a modified IgG1 Fc region comprising the substitution of amino acid residue Glycine (G) at position 236 to Alanine (A), and a fucosylation content of up to 40% out of all glycan structures.

According to some embodiments, the pharmaceutical composition comprises as an active ingredient, at least one antibody that specifically binds GITR, or a conjugate thereof, said antibody comprises a modified IgG1 Fc region comprising the substitution of amino acid residue Glycine at position 236 to Alanine residue (G236A), a substitution of amino acid residue Alanine at position 330 to Leucine residue (A330L), and a substitution of amino acid residue Isoleucine at position 332 to Glutamic acid residue (I332E).

According to some embodiments, the pharmaceutical composition comprises an antibody comprising a set of six CDRs wherein: heavy chain CDR1 comprises SEQ ID NO: 1; heavy chain CDR2 comprises SEQ ID NO: 2; heavy chain CDR3 comprises SEQ ID NO: 3; light chain CDR1 comprises SEQ ID NO: 4; light chain CDR2 comprises SEQ ID NO: 5; and light chain CDR3 comprises SEQ ID NO: 6.

According to some embodiments, the pharmaceutical composition comprises an antibody comprising a set of six CDRs wherein: heavy chain CDR1 comprises SEQ ID NO: 7; heavy chain CDR2 comprises SEQ ID NO: 8; heavy chain CDR3 comprises SEQ ID NO: 9; light chain CDR1 comprises SEQ ID NO: 10; light chain CDR2 comprises SEQ ID NO: 11; and light chain CDR3 comprises SEQ ID NO: 12.

According to a specific embodiment, the pharmaceutical composition comprises an antibody comprising a heavy chain having a sequence selected from SEQ ID NO: 13 and 15 and a light chain having the sequence of SEQ ID NO: 14. Each possibility represents a separate embodiment of the invention.

The pharmaceutical compositions of the present invention may be formulated for administration by any mean suitable for administration of antibodies. According to some embodiments, the pharmaceutical compositions are formulated for intravenous (i.v.) administration. In some embodiments, the pharmaceutical compositions are formulated for administration by injection or by infusion.

Also provided are pharmaceutical compositions, comprising at least one antibody, or antibody conjugate described above, for use in enhancing the immune co-stimulatory activity of hGITR.

According to some embodiments, the pharmaceutical composition comprising at least one Fc-modified antibody to hGITR described above is for use in activating dendritic cells, activating phagocytosis and enhancing production of pro-inflammatory cytokines.

According to some embodiments, the pharmaceutical composition is for use in treatment of cancer or tumor.

According to some embodiments, the tumor is a solid tumor. According to other embodiments, the cancer is a hematological cancer.

According to some embodiments, the cancer is a metastatic cancer.

According to some specific embodiments, the solid cancer is selected from the group consisting of non-small cell lung carcinoma (NSCLC), renal cell carcinoma, melanoma, glioblastoma, colorectal cancer, breast cancer and ovarian carcinoma.

According to some embodiments, treatment of a cancer or a tumor comprises administering or performing at least one additional anti-cancer therapy. According to certain embodiments, the additional anti-cancer therapy is selected from surgery, chemotherapy, radiotherapy, immunotherapy and hormone therapy.

According to some embodiments, treatment of cancer or a tumor comprises administration of the anti hGITR antibody or the conjugate thereof, and an additional anti-cancer agent. According to some embodiments, the additional anti-cancer agent is selected from the group consisting of an immune-modulator, an agent that binds to a tumor antigen or to a receptor over-expressed on tumor cells, and a chemotherapeutic agent.

According to some embodiments, treatment of a cancer or a tumor comprises delaying, slowing, or preventing tumor growth or recurrence.

According to some embodiments, treatment of a cancer or a tumor, results in preventing or reducing formation, growth or spread of metastases in the subject.

According to some embodiments, treatment of a cancer or a tumor, results in enhancing anti-tumor effect.

According to some embodiments, treatment of a cancer or a tumor, results in induction of long-term anti-tumor immunity.

According to some embodiments, treatment of a cancer or a tumor, results in modulating tumor microenvironment by targeting hGITR and by Fc region mediated FcγRIIA and/or FcγRIIIA activation of effector cells.

According to some embodiments, the pharmaceutical composition comprising at least one Fc-modified antibody to hGITR described above is for use in enhancing anti-tumor effect by activating effectors cells via hGITR and via FcγRIIA and/or FcγRIIIA.

According to some embodiments, the pharmaceutical composition comprising at least one Fc-modified antibody to hGITR described above is for use in inducing long-term anti-tumor immunity.

According to some embodiments, the pharmaceutical composition comprising at least one Fc-modified antibody to hGITR described above is for use in depleting or inhibiting regulatory T cells (Tregs).

According to some embodiments, the pharmaceutical composition comprising at least one Fc-modified antibody to hGITR described above is for use in activating dendritic cells.

According to some embodiments, the pharmaceutical composition comprising at least one Fc-modified antibody to hGITR described above is for use in modulating tumor microenvironment by targeting GITR and by Fc region mediated FcγRIIA and/or FcγRIIA activation of effector cells.

According to some embodiments, the pharmaceutical composition comprising at least one Fc-modified antibody to hGITR described above is for use in delaying, slowing, or preventing tumor growth and metastasis formation or spread.

According to yet another aspect and some embodiments, the present invention provides methods of enhancing the activity of hGITR in a subject in need thereof, comprising administering to said subject a therapeutically effective amount of an anti GITR antibody or conjugate thereof or a pharmaceutical composition comprising them, as defined herein, wherein the anti GITR antibody has an agonistic activity.

According to yet another aspect, the present invention provides a method of treating a cancer or tumor comprising administering to a subject in need thereof, a pharmaceutical composition comprising a therapeutically effective amount of an Fc region modified anti-hGITR antibody or conjugate thereof described herein.

According to some embodiments, the method of treating a cancer or a tumor in a subject in need of such a treatment comprises administering or performing at least one additional anti-cancer therapy. According to certain embodiments, the additional anti-cancer therapy is selected from surgery, chemotherapy, radiotherapy, immunotherapy and hormone therapy.

According to some embodiments, the method of treating cancer or tumor comprises administration of the anti hGITR antibody or the conjugate thereof, and an additional anti-cancer agent. According to some embodiments, the additional anti-cancer agent is selected from the group consisting of an immune-modulator, an agent that bind a tumor antigen or a receptor over-expressed on tumor cells, and a chemotherapeutic agent.

According to some embodiments, the immune modulator is a checkpoint inhibitor.

According to some embodiments, the method of treating cancer or tumor in a subject in need thereof results in preventing or reducing formation, growth or recurrence in the subject.

According to some embodiments, the method of treating cancer or tumor comprises delaying, slowing or preventing metastasis formation or spread.

According to some embodiments, the method of treating cancer or tumor comprises enhancing anti-tumor effect.

According to some embodiments, the method of treating cancer or tumor comprises inducing long-term anti-tumor immunity.

According to some embodiments, the method of treating cancer or tumor comprises modulating tumor microenvironment by targeting hGITR and by Fc region mediated FcγRIIA and/or FcγRIIIA activation of effector cells.

According to some embodiments, the present invention provides methods of activating dendritic cells, activating phagocytosis and enhancing production of pro-inflammatory cytokines, the method comprising administration of at least one Fc-modified antibody to hGITR described above.

According to some embodiments, the present invention provides methods of preventing, delaying, depleting or inhibiting Tregs, comprising administering at least one Fc-modified antibody to hGITR described above.

Also provided according to the present invention is a kit comprising an antibody, antibody conjugate or composition described above, being packaged in a packaging material and identified in print, in or on said packaging material.

Methods for producing and purifying the anti GITR antibodies or antibody conjugates of the present invention, are also included within its scope. Any method known in the art to produce, isolate and purify recombinant antibodies may be used.

According to some embodiments, the antibodies are produced by transient transfection of plasmids comprising polynucleotide sequences encoding heavy and light chain pairs, into cells.

According to some embodiments, the method of producing a Fc-modified antibody to GITR, comprises:
(i) transfecting of vectors, plasmids, or constructs comprising polynucleotide sequences encoding heavy and light chain pairs, into cells.
(ii) culturing cells which comprise at least one polynucleotide encoding a heavy or light chain of an antibody under conditions which allow expression of the heavy and/or light chains; and
(iii) recovering said chains from the cells.

According to other embodiments, the antibodies are assembled in the cells, secreted to the cell culture supernatant and then recovered, separated and purified.

According to some embodiments, separation and purification comprises one or more of the steps: collecting the culture medium, centrifuged to pellet cells and any particulate matter, filtration, purification using chromatography and buffer exchange.

According to some embodiments, the chromatography is a protein G chromatography.

According to a specific embodiment, the humanized antibody of antibody fragment is purified to a level of at least 95%, 96%, 97%, 98%, 99% or even higher level purity (less than 5% host cell contaminants w/w or w/v).

Any method known in the art to produce afucosylated antibodies may be used to produce the Fc-afucosylated antibodies of the present invention.

According to some embodiments, afucosylation of the antibodies is performed without modifying an amino acid glycosylation site, e.g., without substituting or deleting the Asparagine (N) amino acid residue at position 297 of the Fc.

According to some embodiments, afucosylation of the antibodies is performed at the transfection step, post translation.

According to some embodiments, the method of producing the afucosylated Fc-modified antibodies comprises adding 50-500 μM of 2-Deoxy-2-fluoro-L-fucose to the transfection medium.

### BRIEF DESCRIPTION OF THE DRAWINGS

FIGS. 1A and 1B are schematic presentations of the FcγR pathways for agonistic anti-GITR antibodies, presenting both activation of the FcγR-pathway (FIG. 1A) and engagement of FcγRs as a scaffold for antibody crosslinking (FIG. 1B).

FIG. 2 is a schematic presentation of an exemplary cloning strategy used to produce some of the antibodies of the present invention. A human IgG1 was cloned and introduced into mammalian expression vectors. The hIgG1, labeled bands of heavy (HC) and light (LC) constant regions were extracted from 1% agarose gel, ligated with heavy and light chain variable regions (VH and VL) of anti-mouse GITR (mGITR), and used for expression and production of Fc region variant antibodies.

FIGS. 3A-3B non-reduced (FIG. 3A) and reduced (FIG. 3B) SDS-PAGE of chimeric rat anti mGITR hIgG1 Fc region variants based on the rat anti mGITR antibody DTA-1. In the non-reduced SDS-PAGE the disulfide bonds between the heavy and the light chains are intact, therefore only one band is apparent at the size of the intact antibody (FIG. 3A), while in the reduced SDS-PAGE (FIG. 3B), two distinct bands appeared: one representing the heavy chain ˜50 kDa and the other for the light chain ˜25 kDa. Lane 1—molecular weight marker, lane 2—anti-mGITR V11, lane 3—afucosylated anti-mGITR, lane 4—N279A, lane 5—GAALIE, lane 6—GASDALIE, lane 7—G236A, lane 8 anti-mGITR IgG1.

FIG. 4 is a graphic presentation showing similar binding affinities of several chimeric anti mGITR hIgG1 Fc-modified variants (comprising the Fab of anti mGITR DTA-1) to plate-bound mouse GITR. ELISA OD450 values were plotted against increasing concentrations of the tested antibody. Data is represented as mean±SEM.

FIGS. 5A-5D are graphs demonstrating the differential binding affinities of chimeric anti mGITR hIgG1 Fc region variants (comprising the Fab of DTA-1) to the indicated hFcγRs; hFcγRIa (FIG. 5A), hFcγRIIA (FIG. 5B), hFcγRIIB (FIG. 5C), and hFcγRIIIA (FIG. 5D). Data is represented as mean±SEM.

FIG. 6 depicts the results of a pharmacokinetics assay for several chimeric anti mGITR IgG1 Fc region variants (comprising the Fab of DTA-1), measured as in-vivo half-life of the antibody. Data is represented as mean±SEM. n=3.

FIGS. 7A-7H present the differential anti-tumor activity of chimeric anti mGITR hIgG Fc region variants. FcγR humanized (hFcγR) mice were inoculated with MC38 cells and treated with chimeric human Fc region variants of DTA-1 antibody. Mice were followed for tumor progression, and overall survival. Untreated mice (n=10) (FIG. 7A), IgG1 treatment (n=8) (FIG. 7B), IgG1-N297A treatment (n=10) (FIG. 7C), IgG1-V11 treatment (n=10) (FIG. 7D), IgG1-G236A treatment (n=9) (FIG. 7E), Afuco-IgG1 treatment (n=10) (FIG. 7F). An unpaired two-tailed t test was used to compare between Fc region variants to IgG1-N297A group (FIG. 7G), comparison of the probability of survival between the Fc region variants (FIG. 7H). Asterisks indicate statistical comparison to IgG1-N297A. Data is represented as mean±SEM.

FIGS. 8A-8B are graphic presentations of the protection provided by anti GITR treatment in tumor re-challenge model as an indication of long-term immunity. Naïve mice and MC38-bearing hFcγR mice treated with anti mGITR Fc region variants that completely respond to previous treatment and become tumor free; IgG1(n=4), IgG1-N297A (n=1), IgG1-G236A (n=6), Afuco-IgG1(n=7) were re-challenged and followed for tumor growth (FIG. 8A) and for probability of survival (FIG. 8B). Asterisks indicate statistical comparison to naïve mice. Data are represented as mean±SEM.

FIGS. 9A-9F depict the long-term protection against re-appearance of a tumor and memory response by anti mGITR mAbs. MC38-bearing hFcγR mice treated with anti-GITR Fc region variants that completely respond to previous treatment and became tumor free were compared with same type of tumor-free mice that were injected with antibodies depleting CD4 and CD8 cells. Comparison of tumor growth of T-cells depleted mice to non T-cells depleted mice for each Fc region variant treatment: untreated (n=6, FIG. 9A), IgG1-G236A (T cells depleted n=4, non T cells depleted n=2, FIG. 9B), Afuco-IgG1 (T cells depleted n=3, non T cells depleted n=2, FIG. 9C), and Afuco-IgG1-G236A (T cells depleted n=2, non T cells depleted n=4, FIG. 9D). Comparisons of tumor growth over time (FIG. 9E) and survival probabilities (FIG. 9F) were made. Asterisks indicate statistical comparison between T cell depleted mice to non T-cell depleted mice, which treated with the same Fc region variant. Data are represented as mean±SEM.

FIGS. 10A-10C are graphic presentations of the binding of the chimeric Fc region modified anti mGITR (DTA-1 Fab) variants to hFcγRIIA (FIG. 10A), hFcγRIIIA (FIG. 10B) and hFcγRIIB (FIG. 10C), measured by ELISA. Data presented as mean±SEM.

FIGS. 11A-11F are graphic presentations of the enhanced FcγRIIA and FcγRIIIA activity mediated by Fc region modified chimeric anti mouseGITR antibodies. Humanized FcγR mice were inoculated with MC38 cells and once tumor reached average volume of 115 mm³ they were treated with the indicated human Fc region variants and monitored for tumor progression: untreated mice (FIG. 11A), IgG1-G236A (FIG. 11B), Afuco-IgG1 (FIG. 11C), Afuco-IgG1-G236A (FIG. 11D), comparison of average tumor growth of all Fc region variants, an unpaired two-tailed t test was used to compare between Afuco-IgG1-G236A to Afuco-IgG1 group (FIG. 11E) and probability of survival (FIG. 11F). Data is represented as mean±SEM.

FIGS. 12A-12F graphic presentation of the enhanced anti-tumor activity of chimeric anti mGITR hIgG Fc region variants. Humanized FcγR Mice were inoculated with MC38 cells once tumor reached average volume of 115 mm³ they were treated with the indicated chimeric antibody variants (DTA-1 based). Mice were followed for tumor progression: untreated mice (FIG. 12A), IgG1 (FIG. 12B), IgG1-N297A (FIG. 12C), Afuco-IgG1-G236A (FIG. 12D), comparison of average tumor growth of all Fc region variants, an unpaired two-tailed t test was used to compare between Afuco-IgG1-G236A to IgG1 group. (FIG. 12E) and probability of survival (FIG. 12F). Data are represented as mean±SEM.

FIGS. 13A-13F present T cells frequencies in the TME, 1-, 4- and 8-days post onset of anti-GITR Fc region variants treatment. Humanized FcγR mice with refractory MC38 tumors were treated with chimeric anti mGITR Fc region variants IgG1-N297A (NA), Afuco-IgG1-G236A (GA-aFuc), and IgG1. Mice were sacrificed and tumors were harvested at three time points following treatment, 1 day, 4 days (total of 2 mAb administrations) and 8 days (total of 3 mAb administrations), untreated mice were used as control. FIGS. 13A-13C, flow cytometry analyses of Treg frequency detected for CD45+CD3+CD11b-CD8-CD4+FoxP3+: 1 day following the onset of treatment (FIG. 13A), 4 days following the onset of treatment (FIG. 13B), and 8 days following the onset of treatment (FIG. 13C). FIGS. 13D-183F, flow cytometry analyses of CD8+frequency detected for CD45+CD3+CD11b−CD8+CD4−: 1 day following the onset of treatment (FIG. 13D), 4 days following the onset of treatment (FIG. 13E), and 8 days following the onset of treatment (FIG. 13F). FIGS. 13G-13I, CD8/Tregs ratios in the TMEs of mice: 1 day following the onset of treatment (FIG. 13G), 4 days following the onset of treatment (FIG. 13H), and 8 days following the onset of treatment (FIG. 13I).

FIGS. 14A-14G depict flow cytometry analyses of DCs activation state within the TMEs and dLNs from humanized FcγR mice with refractory MC38 tumors treated with either Afuco-IgG1-G236A (GA-aFuc), IgG1 Fc-silent N297A (NA), and IgG1. The TMEs and dLNs were harvested 4- and 8-days post treatment onset, untreated mice served as control (untreated). DCs were detected for CD45+ CD11b− NK1.1− F4/80− CD11c+ MHCII+; activated CD80 CDs were detected for CD45+ CD11b− NK1.1− F4/80− CD11c+ MHCII+ CD80+ CD86−, and activated CD86 DCs were detected for CD45+ CD11b− NK1.1− F4/80− CD11c+ MHCII+ CD80− CD86+. FIG. 14A presents, the percentages of DCs in the TMEs 4 days after treatment onset. FIG. 14B presents, the geometric mean fluorescence intensity (gMFI) of activated CD80 DCs in the TMEs 4 days after treatment onset. FIG. 14C presents, gMFI of activated CD86 DCs in the TMEs 4 days after treatment onset. FIG. 14D presents, the percentages of DCs in the dLN 4 days after treatment onset. FIG. 14E presents, the geometric mean fluorescence intensity (gMFI) of activated CD80 DCs in the dLN 4 days after treatment onset. FIG. 14F presents, gMFI of activated CD86 DCs in the dLN 4 days after treatment onset. FIG. 14G presents, the percentages of DCs in TMEs, 8 days post treatment onset.

FIGS. 15A-15C are graphic presentations of differential binding affinities of humanized anti hGITR (TRX518) IgG1 Fc region variants to hGITR and to the indicated hFcγRs. The binding affinities of anti-human IgG1 Fc region variants to hGITR (FIG. 15A) and to human FcγRIIA (FIG. 15B) and FcγRIIIA (FIG. 15C) were evaluated using comparative ELISA. OD450 values were plotted against increasing concentrations of test antibody to assess binding to plate-bound protein. Data is represented as mean±SEM

FIG. 16 is a graphic presentation of the effect of T cell frequencies in blood after treatment with anti hGITR antibody. Humanized FcγR and hGITR mice inoculated with MC38 tumors were treated with humanized Fc region modifies variants of anti hGITR antibodies IgG1-N297A, and Afuco-IgG1-G236A, both comprising Fab of TRX518. Following one administration of mAb, blood was collected at the following time points: 2 days before treatment, and 2, 22 and 96 hours after treatment. Data are represented as mean±SEM and Ordinary one-way ANOVA using Tukey's multiple comparisons statistical test. For all group n=5.

FIGS. 17A-17C present T cell frequencies in the TME following anti hGITR treatments. Humanized FcγR/GITR mice with refractory MC38 tumors were treated with anti hGITR Fc region variants IgG1-N297A (TRX518), and Afuco-IgG1-G236A. Tumors were harvested 4 days (total of 2 mAb administrations) from treatment onset and single cell suspensions were generated and analyzed by flow cytometry for modulation of the immune compartment of the TME of CD4 FoxP3—(FIG. 17A), CD8 (FIG. 17B) and Tregs (FIG. 17C). Data is represented as mean±SEM and Kruskal-Wallis statistical test was performed.

FIG. 18A a timeline of an in vivo NK depletion assay, from the inoculation of refractory MC38 tumor cells into mice to day 4 from the onset of anti-mGITR hIgG1 Fc region variants treatment when blood was drawn and tumors were harvested, including the schedule of NK depletion by αNK1.1. Assessing NK cells is required for the depletion of Tregs by Afuco-IgG1-G236A.

FIGS. 18B-18C flow cytometry of NK cell in the blood samples from mice on days 0 (FIG. 18B) and 4 (FIG. 18C) from onset of Afuco-IgG1-G236A, or IgG1 Fc-silent N297A (NA) treatment and following NK cells depletion. NK cells were detected for CD45+CD3-NKp46+, blood from mice treated with PBS only served as negative control. FIGS. 18D-18F, depict, cells count per mg tumor of: NK cells (FIG. 18D), Treg cells (detected for CD45+CD3+CD11b−CD8−CD4+ FoxP3+, (FIG. 18E), and CD8+ cells (detected for CD45+CD3+CD11b−CD8+CD4−, FIG. 18F).

FIGS. 19A-19C depict ELISA analyses, measuring the binding of novel anti-mGITR bispecific variants to the different hFcγRs, compared to the monospecific Afuco-IgG1-G236A (DTA1 GA-aFuc, circles) which served as a control. Non-agonistic bispecific antibodies were generated based on the Fc scaffolds of (i) Afuco-IgG1-G236A and the Synagsis antibody (GITR/Afuco-IgG1-G236A Syn, DTA1/Syn GA-aFuc, triangles) and (ii) IgG1-N297A and Synagsis (GITR/NA Syn DTA1/NA Syn, upside down triangles), the Abs were assessed by ELISA for their binding to FcγRIIA (FIG. 19A), FcγRIIB (FIG. 19B) and FcγRIIIA (FIG. 19C). FIG. 19D, HPLC characterization of the bispecific anti-GITR GITR/Afuco-IgG1-G236A Synagis non-agonistic antibody digested with trypsin.

FIG. 20A depicts binding capacity of a novel non agonist bispecific anti-mGITR mAb with the N297A substitution in the Fc, denoted “mGITR/NA Synagis” (DTA-1/Synagsis NA, squares) compared to the monospecific mIgG1-N297A (full circles). Non-GITR binding antibody with the N297A substitution in the Fc region as an isotype control (isotype control NA, triangles) The binding capacity was assayed by ELISA, OD450 values were plotted against increasing concentrations of indicated antibody.

FIG. 20B depicts IL-2 secretion by isolated T cells from wild type (WT) mice (activated with anti-CD3 antibodies) following their incubation with increasing concentrations of anti-mGITR/NA Synagis (DTA-1/Synagsis NA, squares), the monospecific mIgG1-N297A (DTA-1 NA full circles), or a non-GITR binding antibody with the N297A substitution in the Fc region (isotype control NA) as an isotype control (triangles). ELISA was performed on the supernatant of the activated T cells incubated with the Abs.

FIG. 21 flow cytometry analysis of the percent of Tregs in the TME of hFcγR mice with MC38 tumors following different treatments. The mice were treated (IP) with 100 μg, 200 μg, or 400 μg of a humanized GA-aFuc Fc region variant of the non-agonistic bispecific Ab (GITR/syn GA-aFuc), or with 100 μg of mouse anti-DTA-1 (anti-GITR) GA-aFuc Ab. Tumors from untreated nice were analyzed as well as a negative control.

FIGS. 22A-22C depicts tumor volume (mm³) progression in hFcγR mice with MC38 tumors following treatment with 100 μg GA-aFuc Ab (full triangles) or with 200 μg of the non-agonistic GITR/syn GA-aFuc Ab (empty triangles). Untreated mice served as a control (full circles). FIG. 22A depicts, 20 days tumor volume measurements. FIG. 22B depicts, tumors volumes of mice of all groups on day 13 from treatment onset. FIG. 22C depicts, tumors volumes of mice of GA-aFuc Ab and GITR/syn GA-aFuc Ab tested groups on day 15 from treatment onset.

FIGS. 23A-23B flow cytometry analyses of the percent of Tregs and dendritic cells (DCs) in the TME of hFcγR mice with MC38 tumor, respectively, following the treatment with 200 μg, of a GITR/syn GA-aFuc (triangles), and with 100 μg GA-aFuc (squares). Tumors from untreated nice were analyzed as well as a negative control (circles). Tregs were detected by CD45+CD3+CD11b−CD8−CD4+FoxP3+ and DCs were detected by CD45+ CD11b− NK1.1− F4/80− CD11c+ MHCII+.

FIGS. 23C-23D flow cytometry analyses of the activation of DCs by detecting the percent of CD80+ or CD86+ expressing DCs, respectively. Tumors from untreated nice were analyzed as well as a negative control. DCs were detected for by CD45+ CD11b− NK1.1− F4/80− CD11c+ MHCII+, CD80+ DCs were detected with the addition of anti-CD80, CD86+ DCs were detected with the addition of anti-CD86.

FIG. 24A depicts 17 days tumor volume progression surveillance in wild type WT C57BL/6J mice with MC38 tumors following treatment with 100 μg DTA-1 mIgG2a (squares) or with 100 μg of anti-GITR-mIgG2a N279A Ab (DTA-1 mIgG2a NA, empty triangles). Untreated mice served as a control (full circles). FIG. 24B-24E, flow cytometry analyses for modulation in the immune compartment in the TME of WT mice with MC38 tumors were treated with 100 μg of DTA-1 mIgG2a Ab (squares) and 100 μg of DTA-1 mIgG2a NA (triangles) or of untreated mice (circles) used as a control. Tumors were harvested 4 days post treatment and analyzed. FIG. 24B, Tregs count/mg tumor detected using anti-FoxP3 in all tested groups. FIG. 24C, DCs percentages out of total lymphocyte (detected using anti-CD11c/anti-CD45) in all tested groups. FIG. 24D, delta gMFI of CD80+ activated DCs in all tested groups. FIG. 24E, delta gMFI of CD86+ activated DCs in all tested groups.

FIG. 25, a graphic presentation of tumor volume (mm³) measurements for 13 days in BATF3^{-/-} mice with MC38 tumors treated with 100 μg of DTA-1 mIgG2a (squares) Ab and 100 μg DTA-1 mIgG2a-NA Ab (triangles), or untreated mice (circles) used as control.

FIGS. 26A-26B, graphic presentations of tumor volume (mm³) progression in XCR1-iDTR and ZBTB46-iDTR NC38 tumor bearing mice treated with 100 μg DTA-1 mIgG2a Ab, 20 ng/gr body weight Diphtheria Toxin (DT), or the combination of 100 μg DTA-1 mIgG2a Ab and 20 ng/gr body weight DT, respectively. Untreated mice were used as control. The DT was injected every other day 20 ng/g, to deplete DCs. FIG. 26A 13 days tumor volume (mm³) progression in XCR1-iDTR XCR1-iDTR mice, a DT mediated inducible cDC1 cells depleting model. FIG. 26B 20 days tumor volume (mm³) progression in ZBTB46-iDTR mice, a DT mediated inducible cDC1 and cDC2 cells depleting model.

FIGS. 27A-27B, percentage of Tregs in the TME and percentage of DCs in the dLN, respectively, from MC38 tumor bearing mice treated with 100 μg of mouse anti-GITR Ab, 100 μg of anti-CD25 Ab, or of untreated mice. The organs were harvested 4 days post treatment.

FIGS. 27C-27D, depict the delta gMFI of CD80+ and CD86+ activated DCs, respectively, in dLNs from mice treated with 100 μg of mouse anti-GITR Ab, 100 μg of anti-CD25 Ab, or of untreated mice. The dLNs were harvested 4 days post treatment. FIG. 27C depicts, the delta gMFI of CD80+ activated DCs. FIG. 27D depicts, the delta gMFI of CD86+ activated DCs.

FIGS. 28A-28C, depict tumor volume progression in MC38 tumor bearing mice treated three times with 100 μg/mice of IgG1-N297A (N297A), or IgG1-GAALIE (GAALIE). Untreated mice were used as control. Each line represents one animal.

FIG. 28D, graphic presentation of average tumor volume progression for each treatment group IgG1-N297A (N297A, n=8), IgG1-GAALIE (GAALIE, n=9), control, untreated mice (untreated n=8). An unpaired two-tailed t test was used to compare between IgG1-N297A and IgG1-GAALIE groups. Data are represented as mean±SEM

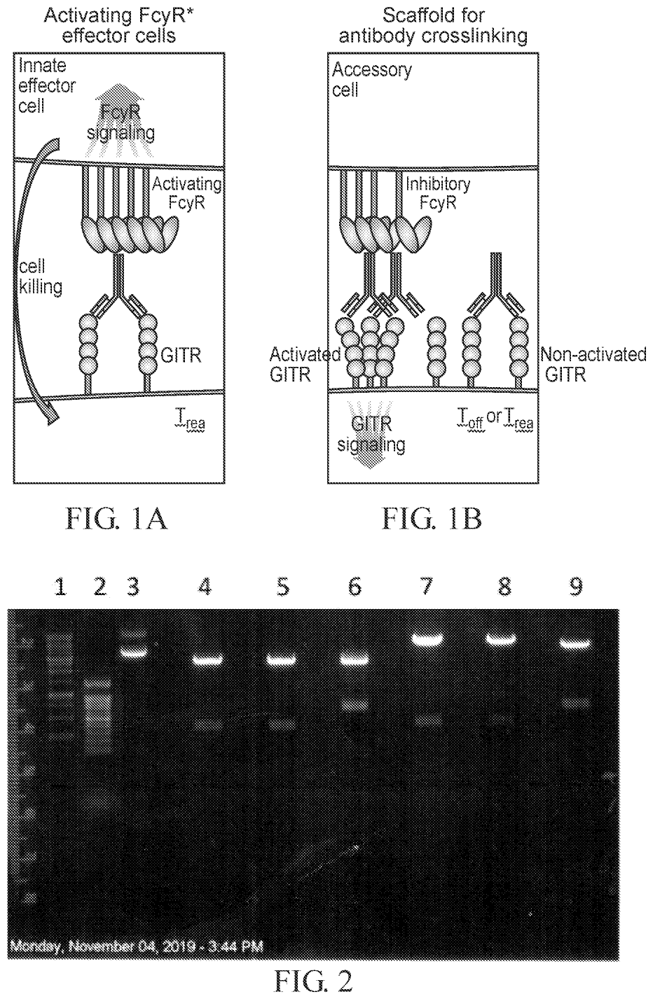

Any embodiment disclosed herein above can optionally be combined with the subject matter of one or any combination of another embodiment disclosed herein. Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### DETAILED DESCRIPTION OF THE INVENTION

The present invention provides antibodies, specific to human protein Glucocorticoid-induced TNFR-related (hGITR, also termed CD357 or TNFRSF18) that comprises modified human IgG1 Fc, that exhibited enhanced binding ratio to activating FcγRIIA and/or FcγRIIIA relative to binding to FcγRIIB, augmenting control of tumor growth and overall survival. The Fc-modified anti hGITR antibodies of the present invention were designed by combining two strategies-glyco-engineering, namely altering the content of the sugar moieties (e.g., afucosylation), and protein engineering by substitutions of one or more amino acids (e.g., G236A). In the process of developing the anti GITR antibodies of the present invention, initial experiments were performed in human FcγR mice, testing chimeric anti mouse GITR antibodies comprising human IgG1 Fc region and variants thereof. Following promising anti-tumor activity, anti hGITR antibodies comprising the modified hIgG1 Fc region were generated and tested in human FcγR-human GITR mice models.

The enhanced affinity towards FcγRIIA and/or FcγRIIIA relative to the decreased or unchanged binding affinity towards FcγRIIB by the antibodies of the present invention results in a significant increase of the Activating/Inhibitory ratio. This ratio directly correlates with the ability of the antibodies to stimulate the immune system via activation of dendritic cells, phagocytosis, and production of pro-inflammatory cytokines which in turn elicit anti-tumor effect and long-term anti-tumor immunity. Surprisingly, the Fc-modified antibodies of the present invention, enhance anti-tumor immune activity without the need of activating the GITR receptor.

Without wishing to be bound to any theory of mechanism of action, it is suggested that the decrease in binding of the Fc-modified IgG1 of anti hGITR antibodies of the present invention to FcγRIIB, relative to binding to the activating receptors, impairs the crosslinking activity of the antibodies. Antibody crosslinking mechanism, through binding to FcγRIIB is known for TNFR family members for its role in enhancing the agonistic activity. Surprisingly, the Fc-modified anti hGITR antibodies of the present invention demonstrate significant enhancement of anti-tumor activities even without such crosslinking.

Compared to antibodies comprising substantially the same antigen binding site (e.g., antibodies herein termed “IgG1”, “IgG1-N297A” or “V11”), the antibodies of the present invention (e.g., antibodies termed “afucosylatedIgG1-G236A” or “afuco-G236A”, and “GAALIE”) exhibited enhanced binding to both FcγRIIA and FcγRIIIA activating receptors, relative to binding to the inhibitory receptor FcγRIIB. The “VII” Fc variant which exhibits high affinity towards the FcγRIIB, failed to elicit anti-tumor effect and long-term anti-tumor immunity.

The anti hGITR antibodies of the present invention decrease Treg frequency in the TME by Fc-mediated depletion mechanism. CD4 Tregs decrease occurs at early time point with the Fc-modified Afuco-G236A antibody. Anti hGITR-mediated Treg depletion is FcγR dependent. Afuco-G236A anti hGITR antibody of the present invention demonstrated an FcγR mediated-Treg depletion, while the antibody TRX518, currently in clinical trials, that is an Fc region silent antibody does not mediate Treg depletion. Moreover, The Afuco-G236A Fc-modified variant of the present invention demonstrated the highest increase in density of both DCs activating markers, CD80 and CD86 within the tumor and draining lymph nodes (dLN), among all Fc region variants tested. The IgG1 Fc-silent N297A showed no increase in CD80 or CD86 markers.

Once again, unexpectedly, the Fc-modified antibodies of the present invention although mainly engaging activating receptors, but do not enhance crosslinking, demonstrate increased anti-cancer activity compared to similar antibodies having different Fc scaffolds.

### Definitions

The term “glucocorticoid-induced TNFR family-related receptor (abbreviated herein as “GITR”), also known as TNF receptor superfamily 18 (TNFRSF18), AITR, CD357, ENERGEN, or GITR-D, as used herein, refers to a member of the tumor necrosis factor/nerve growth factor receptor family. The human member is a type I transmembrane protein characterized by three cysteine pseudorepeats in the extracellular domain (Nocentini, G. et al. (1997) Proc. Natl. Acad. Sci., USA 94:6216-622). The Fc-modified antibodies of the present invention may be directed against any human GITR protein variant expressed on human cancerous cells and/or on human immune cells. Non limiting examples for human protein variants include accession number NM_004195.3, 241 amino acids protein of accessions NP_004186.1 and GI: 4759246, 255 amino acids protein of accessions NP_683699.1 and GI: 23238194, and 234 amino acids protein of accessions NP_683700.1 and GI: 23238197. Non-limiting exemplary mouse GITR protein variants accession numbers are NM_021985.3, 132 amino acids protein of accessions NP_068820.1 and NM_009400.3, 228 amino acids protein of accessions NP_033426.1.

Antibodies, or immunoglobulins, comprise two heavy chains linked together by disulfide bonds and two light chains, each light chain being linked to a respective heavy chain by disulfide bonds in a “Y” shaped configuration. Each heavy chain has at one end a variable domain (V_{H}) followed by a number of constant domains (C_{H}). Each light chain has a variable domain (V_{L}) at one end and a constant domain (C_{L}) at its other end, the light chain variable domain being aligned with the variable domain of the heavy chain and the light chain constant domain being aligned with the first constant domain of the heavy chain (CH1). The variable domains of each pair of light and heavy chains form the antigen-binding site (Fab). The domains on the light and heavy chains have the same general structure and each domain comprises four framework regions, whose sequences are relatively conserved, joined by three hyper-variable domains known as complementarity determining regions (CDRs). These domains contribute specificity and affinity of the antigen-binding site.

“Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273, 927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January, 27(1):55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309(3):657-70, (“Aho” numbering scheme); and Whitelegg N R and Rees A R, “WAM: an improved algorithm for modelling antibodies on the WEB,” Protein Eng. 2000 December; 13(12):819-24 (“AbM” numbering scheme.

There are several methods known in the art for determining the CDR sequences of a given antibody molecule, but there is no standard unequivocal method. Determination of CDR sequences from antibody heavy and light chain variable regions can be made according to any method known in the art, including, but not limited to, the Kabat Chothia, and IMGT methods. A selected set of CDRs according to the present invention may include sequences identified by more than one method, namely, some CDR sequences may be determined using Kabat and some using IMGT, for example. According to some embodiments, the CDR sequences of the antibody variable regions are determined using the Kabat and/or Chothia methods. In certain embodiments the CDRs of the antibodies described herein can be defined by a method selected from Kabat, Chothia, IMGT, Aho, AbM, or combinations thereof. In some embodiments, the CDRs are defined using Kabat.

Alterations (e.g., substitutions) may be made in CDRs, e.g., to improve antibody affinity. Such alterations may be made in CDR encoding codons with a high mutation rate during somatic maturation (e.g., Chowdhury, Methods Mol. Biol. 207:179-196 (2008)), and the resulting variant can be tested for binding affinity. Affinity maturation (e.g., using error-prone PCR, chain shuffling, randomization of CDRs, or site/oligonucleotide-directed mutagenesis) can be used to improve antibody affinity (e.g., Hoogenboom et al. in Methods in Molecular Biology 178:1-37 (2001)). CDR residues involved in antigen binding may be specifically identified, e.g., using alanine scanning mutagenesis or modeling (e.g., Cunningham and Wells Science, 244:1081-1085 (1989)). CDR-H3 and CDR-L3 in particular are often targeted.

Further included within the scope of the invention are chimeric antibodies; human and humanized antibodies; recombinant and engineered antibodies, and conjugates thereof. Furthermore, the DNA encoding the variable region of the antibody can be inserted into the DNA encoding other antibodies to produce chimeric antibodies.

The antibodies herein specifically include “chimeric” antibodies. Chimeric antibodies are molecules, the different portions of which are derived from different animal species, such as those having a variable region derived from a murine mAb and a human immunoglobulin constant region. Antibodies which have variable region framework residues substantially from human antibody and complementarity determining regions substantially from a mouse antibody are also referred to as humanized antibodies. Chimeric antibodies are primarily used to reduce immunogenicity in application and to increase yields in production, for example, where murine mAbs have higher yields from hybridomas but higher immunogenicity in humans, such that human/murine chimeric mAbs are used. Chimeric antibodies and methods for their production are known in the art (for example PCT patent applications WO 86/01533, WO 97/02671, WO 90/07861, WO 92/22653 and U.S. Pat. Nos. 5,693,762, 5,693,761, 5,585,089, 5,530,101 and 5,225,539).

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody typically comprises a modified human immunoglobulin constant region (Fc).

Non-human antibodies may be humanized by any methods known in the art. In one method, the non-human complementarity determining regions (CDRs) are inserted (grafted) into a human antibody or consensus antibody framework sequence. Further changes can then be introduced into the antibody framework to modulate affinity or immunogenicity.

For example, U.S. Pat. No. 5,585,089 of Queen et al. discloses a humanized immunoglobulin and methods of preparing same, wherein the humanized immunoglobulin comprises complementarity determining regions (CDRs) from a donor immunoglobulin and heavy and light chain variable region frameworks from human acceptor immunoglobulin heavy and light chains, wherein said humanized immunoglobulin comprises amino acids from the donor immunoglobulin framework outside the Kabat and Chothia CDRs, wherein the donor amino acids replace corresponding amino acids in the acceptor immunoglobulin heavy or light chain frameworks. U.S. Pat. No. 5,225,539, of Winter, also discloses an altered antibody or antigen-binding fragment thereof and methods of preparing same, wherein a variable domain of the antibody or antigen-binding fragment has the framework regions of a first immunoglobulin heavy or light chain variable domain and the complementarity determining regions of a second immunoglobulin heavy or light chain variable domain, wherein said second immunoglobulin heavy or light chain variable domain is different from said first immunoglobulin heavy or light chain variable domain in antigen binding specificity, antigen binding affinity, species, class or subclass.

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art. In one embodiment, the human antibody is selected from a phage library, where that phage library expresses human antibodies (Vaughan et al. Nature Biotechnology 1996 14, 309-314; Sheets et al. PNAS (USA), 1998, 95, 6157-6162); Hoogenboom and Winter, J. Mol. Biol., 1991, 227, 381; Marks et al., J. Mol. Biol., 1991, 222, 581). Human antibodies can also be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al, Bio/Technology 10: 779-783 (1992); Lonberg et al., Nature 368: 856-859 (1994); Morrison, Nature 368:812-13 (1994); Fishwild et al., Nature Biotechnology 14: 845-51 (1996); Neuberger, Nature Biotechnology 14: 826 (1996); Lonberg and Huszar, Intern. Rev. Immunol. 13:65-93 (1995). Alternatively, the human antibody may be prepared via immortalization of human B lymphocytes producing an antibody directed against a target antigen (such B lymphocytes may be recovered from an individual or may have been immunized in vitro). See, e.g., Cole et al., Monoclonal Antibodies and Cancer Therapy, Alan R. Liss, p. 77 (1985); Boerner et al., J. Immunol., 147(1):86-95 (1991); and U.S. Pat. No. 5,750,373.

The term “site-directed mutagenesis” as used herein refers to an in vitro procedure that uses custom designed oligonucleotide primers to confer a desired mutation in a double-stranded DNA plasmid. The most widely-used methods incorporate mutations into the plasmid by inverse PCR with standard primers. For example, one of those methods utilizes overlapping primers special affinity designed to introduce the specific desired substitution.

Affinity is the strength of binding of a single molecule to its ligand. It is typically measured and reported by the equilibrium dissociation constant (K_{D}), which is used to evaluate and rank order strengths of bimolecular interactions. The binding of an antibody to its antigen is a reversible process, and the rate of the binding reaction is proportional to the concentrations of the reactants.

K_{D} is the equilibrium dissociation constant, a ratio of k_{off}/kₒₙ, between the antibody and its antigen. K_{D} and affinity are inversely related. The K_{D} value relates to the concentration of antibody (the amount of antibody needed for a particular experiment) and so the lower the K_{D} value (lower concentration) and thus the higher the affinity of the antibody.

Ki refers to an inhibition constant, used to describe the binding affinity of molecule to its ligand (e.g., Antibody and antigen, enzyme and ligand). Ki also represents dissociation constant as K_{D}, but more narrowly for the binding of a ligand whose binding reduces the activity of the binding molecule. The binding equilibrium described by the Ki value depends on the kinetic mechanism of inhibition.

Ki is measured through inhibition kinetics, for a non-limiting example by competitive ELISA, while ‘K_{D}’ is preferred when the binding is measured more directly, for example by surface plasmon resonance (SPR) (Scarano S, Mascini M, Turner A P, Minunni M. Surface plasmon resonance imaging for affinity-based biosensors. Biosens Bioelectron. 2010, 25: 957-66). A lower K_{D} and Kᵢ values reflect higher affinity and inhibition.

The Fc region of monoclonal antibodies acts as an important bridge between adaptive and innate immune response. When antigens expressed on the surfaces of cancer cells, virus-infected cells or invading pathogens are recognized by specific antibodies, the cells or pathogens become coated with the antibodies. The Fc region of the antibodies bound to these surfaces assists in the elimination of the targets via different mechanisms. Firstly, it can interact with the C1 molecule of the complement system and trigger the activation of classical pathway of the complement system. It can also recruit phagocytes via Fc receptors and activate the phagocytosis (ADCP) pathway and, ADCC mediated by NK cells macrophages and additional effector cells. Among these mechanisms, studies on rituximab (anti CD20) and trastuzumab (anti HER2/neu, also known as Herceptin) have suggested that ADCC/ADCP are the key mechanisms of action to eliminate cancer cells.

As used hereinafter, the term “Activation of dendritic cells (DCs)” refers to the process triggered by pathogens, inflammatory stimuli, or by T helper lymphocytes. Activation of DCs sets multiple changes, enabling the activated DCs transform into the most efficient antigen-presenting cells. One of those changes is the marked increase in the surface expression of co-stimulatory molecules for T cells, such as CD80 and CD86, which serve as a marker for activated DCs.

The Fc region modifications of the present invention may be made by site/oligonucleotide-directed mutagenesis leading to substitution, addition or deletion of at least one amino acid residue of the polypeptide or executed on the glycosylation level of the Fc region, e.g., by removal or addition of at least one oligosaccharide (N-glycan) moiety, or more specifically by reduction of fucose units (afucosylation).

As used herein after the term “Afucosylated antibody” refers to an antibody that its Fc region carries significantly reduced, namely more than 50% reduction, or no fucose on the glycan structure, relative to a parent antibody comprising an unmodified IgG1.

Significant reduced fucosylation according to some embodiments of the present invention refers to a fucose content or level of up to about 40% out of all glycan structures. According to some embodiments, the antibody comprises 20-40% fucose, out of all glycan structures, on its Fc region.

Afucosylation, namely reduction of the fucose content in an antibody Fc, may be performed by any method know in the art, including in cell lines (e.g., CHO cells) in which the fucosylation mechanism is either genetically knocked out, blocked, or altered. According to some embodiments, afucosylation is performed post-translationally.

Afucosylation may be also performed according to any one of the following, non-limiting approaches:
(i) Fucosyltransferase (FUT) inhibitors, e.g., the L-fucose analog, 2-fluoro peracetylated fucose (2FF), which interferes with the normal synthesis of GDP-fucose and inhibits core fucosylation (Mishra N et al., “Comparison of two glycoengineering strategies to control the fucosylation of a monoclonal antibody”. J Biotechnol. 2020; Zhou, Y., et al., “Inhibition of fucosylation by 2-fluorofucose suppresses human liver cancer HepG2 cell proliferation and migration as well as tumor formation”. Sci Rep 7, 11563 (2017).
(ii) Lec13 cells as the host cell line for the production of afucosylated antibodies (Shields R. L., et al., Lack of fucose on human IgG1 N-linked oligosaccharide improves binding to human Fcgamma RIII and antibody-dependent cellular toxicity. J Biol Chem. 2002, 277:26733-40).
(iii) A GDP-keto-6-deoxymannose 3,5-epimerase/4 reductase (FX)-knockout CHO cell line to produce antibodies with completely afucosylated N-glycans (Louie S., et al., FX knockout CHO hosts can express desired ratios of fucosylated or afucosylated antibodies with high titers and comparable product quality. Biotechnol Bioeng. 2017, 114:632-44)
(iv) FUT8 (fucosyltransferase 8) −/− cell line which shown to express completely afucosylated antibodies compared to the same antibody produced in the parental cell line (Yamane-Ohnuki, N. et al., Establishment of FUT8 knockout Chinese hamster ovary cells: An ideal host cell line for producing completely defucosylated antibodies with enhanced antibody-dependent cellular cytotoxicity. Biotechnology and bioengineering, 87, 614-622).
(v) rat hybridoma YB2/0 cells with lower levels of the Fut8 (fucosyltransferase 8) mRNA than CHO cells (Shinkawa T. et al., The absence of fucose but not the presence of galactose or bisecting N-acetylglucosamine of human IgG1 complex-type oligosaccharides shows the critical role of enhancing antibody-dependent cellular cytotoxicity. J Biol Chem. 2003 Jan. 31, 278(5):3466-73).
(vi) FUT8 siRNA CHO cells (Mori K. et al., Engineering Chinese hamster ovary cells to maximize effector function of produced antibodies using. Biotechnol Bioeng, 2004, 88:901-8).
(vii) CHO-gmt3 (CHO-glycosylation mutant3) cells which showed a complete lack of core fucose on N-glycans attached to the EPO-Fc fusion protein and IgG1 antibodies produced in the CHO-gmt3 cells (Chan K. F. et al., Inactivation of GDP-fucose transporter gene (Slc35cl) in CHO cells by ZFNs, TALENs and CRISPR-Cas9 for production of fucose-free antibodies. Biotechnol J. 2016, 11:399-414),
(viii) CHO cells that overexpress both GnT-III and αManII which give rise to the highest level of bisecting and afucosylated glycans on IgG antibodies (Ferrara C. et al., Modulation of therapeutic antibody effector functions by glycosylation engineering: influence of Golgi enzyme localization domain and co-expression of heterologous beta1, 4-N-acetylglucosaminyltransferase III and Golgi alpha-mannosidase II. Biotechnol Bioeng, 2006, 93:851-61).
(ix) Small molecules inhibiting antibody fucosylation to complement existing platforms that involve genetic engineering of cell lines for the production of afucosylated antibodies, Okeley et al., Development of orally active inhibitors of protein and cellular fucosylation. Proc Natl Acad Sci USA, 2013, 110:5404-9).
(x) Alternative expression platforms such as plants (Loos A. IgG-Fc glycoengineering in non-mammalian expression hosts. Arch Biochem Biophys, 2012, 526:167-73).

According to some embodiments, a fucosyilation is performed by adding 20-600 μM of 2-Deoxy-2-fluoro-L-fucose to the transfection medium.

Antibody fucose content and afucosylation rate may be determined by any method know in the art, including but not limited to mass spectrometry methods.

Sequence identity is the percentage of amino acids or nucleotides which match exactly between two different sequences. Sequence similarity permits conservative substitution of amino acids to be determined as identical amino acids. The polynucleotide sequences described herein may be codon-optimized for expression in specific cells, such as human cells. Codon optimization does not change the encoded amino acid sequences of the antibody's chain but may, for example, increase the expression in cells.

Variants, analogs and derivatives of the antibody sequences are also within the scope of the present application. These include, but are not limited to, conservative and non-conservative substitution, insertion and deletion of amino acids within the sequence. Such modification and the resultant antibody analog or variant are within the scope of the present invention as long as they confer, or even improve the binding profile to hGITR and to human FcγRs.

The term “antibody variant” as used herein refers to an antibody derived from another antibody by one or more conservative amino acid substitutions and/or by change in the glycosylation content of its Fc region. Variants according to the invention may be made that conserve the overall molecular structure of the encoded proteins. Given the properties of the individual amino acids comprising the disclosed protein products, some rational substitutions will be recognized by the skilled worker.

Conservative substitutions of amino acids as known to those skilled in the art are within the scope of the present invention. Conservative amino acid substitutions include replacement of one amino acid with another having the same type of functional group or side chain, e.g., aliphatic, aromatic, positively charged, negatively charged. These substitutions may enhance oral bioavailability, penetration, and targeting to specific cell populations, immunogenicity, and the like. One of skill will recognize that individual substitutions, deletions or additions to a peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. For example, according to one table known in the art, the following six groups each contain amino acids that are conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

The term “antibody conjugate” as used herein refers to any molecule comprising the antibody of the present invention. For example, fusion proteins in which the antibody is linked to another entity, such as an anti-cancer drug or an identifiable moiety, is considered an antibody conjugate.

The term “nucleic acid” refers to single-stranded or double-stranded sequence (polymer) of deoxyribonucleotides or ribonucleotides. In addition, the polynucleotide includes variants of natural polynucleotides, unless specifically mentioned. According to an embodiment, the nucleic acid may be” selected from the group consisting of deoxyribonucleic acid (DNA), ribonucleic acid (RNA), peptide nucleic acid (PNA), locked nucleic acid (LNA), and analogs thereof, but is not limited thereto. The term encompasses DNA, RNA, single-stranded or double-stranded and chemical modifications thereof.

The term “polynucleotide” as used herein refers to a long nucleic acid comprising more than 150 nucleotides.

The terms “nucleic acid” and “polynucleotide” are used interchangeably herein.

The antibodies described herein are encoded by a nucleic acid. In certain embodiments, the nucleic acid is a component of a vector that can be used to transfer the polypeptide encoding polynucleotide into a cell. As used herein, the term “vector” refers to a nucleic acid molecule capable of transporting another nucleic acid to which it has been linked. One type of vector is a genomic integrated vector, or “integrated vector,” which can become integrated into the chromosomal DNA of the host cell. Another type of vector is an “episomal” vector, e.g., a nucleic acid capable of extra-chromosomal replication. Vectors capable of directing the expression of genes to which they are operatively linked are referred to herein as “expression vectors”. Suitable vectors comprise plasmids, bacterial artificial chromosomes, yeast artificial chromosomes, viral vectors and the like. In the expression vectors regulatory elements such as promoters, enhancers, polyadenylation signals for use in controlling transcription can be derived from mammalian, microbial, viral or insect genes. The ability to replicate in a host, usually conferred by an origin of replication, and a selection gene to facilitate recognition of transformants may additionally be incorporated. Vectors derived from viruses, such as lentiviruses, retroviruses, adenoviruses, adeno-associated viruses, and the like, may be employed. Plasmid vectors can be linearized for integration into a chromosomal location. Vectors can comprise sequences that direct site-specific integration into a defined location or restricted set of sites in the genome (e.g., AttP-AttB recombination). Additionally, vectors can comprise sequences derived from transposable elements.

The nucleic acids encoding the antibodies described herein can be used to infect, transfect, transform, or otherwise render a suitable cell transgenic for the nucleic acid, thus enabling the production of antibodies for commercial or therapeutic uses. Standard cell lines and methods for the production of antibodies from a large-scale cell culture are known in the art. In certain embodiments, the cell is a Eukaryotic cell. In certain embodiments, the Eukaryotic cell is a mammalian cell. In certain embodiments, the mammalian cell is a cell line useful for producing antibodies is a Chines Hamster Ovary cell (CHO) cell, an NS0 murine myeloma cell, or a PER.C6® cell. In certain embodiments, the nucleic acid encoding the antibody is integrated into a genomic locus of a cell useful for producing antibodies. In certain embodiments, described herein is a method of making an antibody comprising culturing a cell comprising a nucleic acid encoding the antibody under conditions in vitro sufficient to allow production and secretion of said antibody.

Any method know for production of recombinant antibodies may be used to produce the antibodies of the present invention. According to one method, cells transformed with nucleotide sequences encoding antibody polypeptides are cultured under effective conditions, which allow for the expression of high amounts of the recombinant polypeptide or polypeptides. Effective culture conditions include, but are not limited to, effective media, bioreactor, temperature, pH and oxygen conditions that permit protein production. An effective medium refers to any medium in which a cell is cultured to produce the recombinant polypeptide of the present invention. Such a medium typically includes an aqueous solution having assimilable carbon, nitrogen and phosphate sources, and appropriate salts, minerals, metals and other nutrients, such as vitamins. Cells of the present invention can be cultured in conventional fermentation bioreactors, shake flasks, test tubes, microtiter dishes and petri plates. Culturing can be carried out at a temperature, pH and oxygen content appropriate for a recombinant cell. Such culturing conditions are within the expertise of one of ordinary skill in the art.

Depending on the vector and host system used for production, resultant polypeptides of the present invention may either remain within the recombinant cell, secreted into the fermentation medium, secreted into a space between two cellular membranes; or retained on the outer surface of a cell or viral membrane.

Following a predetermined time in culture, recovery of the recombinant antibody is affected, e.g., by collecting the whole fermentation medium containing the antibody polypeptide/s, with or without additional steps of separation or purification.

If the antibody polypeptide is expressed in the cell, the cell membrane is preferably disrupted so as to release the polypeptide, using methods known in the art including homogenization.

Any method known in the art for expressing and purifying antibodies may be used to produce the antibodies of the present invention, including but not limited to the method described in Vazquez-Lombardi et al., 2018, nature Protocols, 13, 1, 99-117.

It should be emphasized that different sequencing methods employed on the same protein or nucleotide sequence may result in slightly different sequences due to technical issues and different primers, particularly in the sequence terminals.

Notwithstanding the above, antibodies, of some embodiments of the invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, protein A/G/L separation, mix mode chromatography, metal affinity chromatography, Lectins affinity chromatography, chromatofocusing and differential solubilization.

### Pharmacology and Methods of Treatments

In pharmaceutical and medicament formulations, the active agent is preferably utilized together with one or more pharmaceutically acceptable carrier(s) and optionally any other therapeutic ingredients. The carrier(s) must be pharmaceutically acceptable in the sense of being compatible with the other ingredients of the formulation and not unduly deleterious to the recipient thereof. The active agent is provided in an amount effective to achieve the desired pharmacological effect, as described above, and in a quantity appropriate to achieve the desired exposure.

The antibodies of the present invention as active ingredients are dissolved, dispersed or admixed in an excipient that is pharmaceutically acceptable and compatible with the active ingredient as is well known. Suitable excipients are, for example, water, saline, phosphate buffered saline (PBS), dextrose, glycerol, ethanol, or the like and combinations thereof. Other suitable carriers are well known to those skilled in the art. In addition, if desired, the composition can contain minor amounts of auxiliary substances such as wetting or emulsifying agents, pH buffering agents.

According to some embodiments, the pharmaceutical composition comprises 1-50 mg/ml of an anti-hGITR antibody. According to some embodiments, the pharmaceutical composition comprises a basic amino acid. According to some embodiments, the pharmaceutical composition comprises a sugar. According to some embodiments, the pharmaceutical composition comprises a surfactant. According to some embodiments, the pharmaceutical composition comprises a basic amino acid, a sugar and a surfactant. According to some embodiments, the pharmaceutical composition comprises (i) 1-10 mg/ml of basic amino acid; (ii) 10-200 mg/ml of a sugar; (iii) 0.01-1 mg/ml of a surfactant; (iv) 1-50 mg/ml of anti-hGITR antibody.

According to some embodiments, the basic amino acid is selected from the group consisting of: Histidine, Arginine, Lysine and Ornithine. Each possibility represents a separate embodiment of the present invention.

The term “sugar” refers to monosaccharides, disaccharides, and polysaccharides, Examples of sugars include, but are not limited to, sucrose, trehalose, dextrose, and others. According to some embodiments, the sugar is selected from the group consisting of: sucrose, trehalose, glucose, dextrose and maltose. Each possibility represents a separate embodiment of the present invention. According to some embodiments, the composition comprises 10-200, 10-100, 50-150 or 70-100 mg/ml of sugar. Each possibility represents a separate embodiment of the present invention.

According to yet other embodiments, the composition comprises polyol, including but not limited to mannitol and sorbitol.

According to some embodiments, the surfactant is a non-anionic. According to some embodiments, the surfactant selected from the group consisting of: polysorbates, sorbitan esters and poloxamers. Each possibility represents a separate embodiment of the present invention. According to some embodiments, the composition comprises 0.01-10, 0.01-1, 0.05-5 or 0.1-1 mg/ml of surfactant. Each possibility represents a separate embodiment of the present invention.

Typically, the antibodies and conjugates thereof of the present invention will be suspended in a sterile saline solution for therapeutic uses. The pharmaceutical compositions may alternatively be formulated to control release of active ingredient or to prolong its presence in a patient's system. Numerous suitable drug delivery systems are known and include, e.g., implantable drug release systems, hydrogels, hydroxymethylcellulose, microcapsules, liposomes, microemulsions, microspheres, and the like. Controlled release preparations can be prepared through the use of polymers to complex or adsorb the molecule according to the present invention. For example, biocompatible polymers include matrices of poly(ethylene-co-vinyl acetate) and matrices of a polyanhydride copolymer of a stearic acid dimer and sebaric acid. The rate of release of the molecule according to the present invention, i.e., of an antibody, from such a matrix depends upon the molecular weight of the molecule, the amount of the molecule within the matrix, and the size of dispersed particles.

The pharmaceutical compositions of this invention are formulated for administration by any suitable means, such as intravenously, subcutaneously, intramuscularly, orally, topically, intranasally, intra-arterially, intraarticulary, intralesionally, intratumorally or parenterally. Ordinarily, intravenous (i.v.) administration is used for delivering antibodies. In some embodiments, the antibodies or antibody conjugates are administered by infusion.

According to an aspect, the present invention provides a method of treating cancer comprising administering to a subject in need thereof, a therapeutically effective amount of a pharmaceutical composition comprising an antibody or antibody conjugate described herein.

As used herein the term “subject”, “individual”, or “patient” refers to individuals diagnosed with, suspected of being afflicted with, or at-risk of developing at least one disease for which the described compositions and method are useful for treating. According to some embodiments the individual is a mammal. According to some embodiments, the individual is a human.

It will be apparent to those of ordinary skill in the art that the therapeutically effective amount of the molecule according to the present invention will depend, inter alia upon the administration schedule, the unit dose of molecule administered, whether the molecule is administered in combination with other therapeutic agents, the immune status and health of the patient, the therapeutic activity of the molecule administered, its persistence in the blood circulation, and the judgment of the treating physician.

As used herein the term “therapeutically effective amount” refers to an amount of a drug effective to treat a disease or disorder in a mammal. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the duration of survival, time to disease progression (TTP), the response rates (RR), duration of response, and/or quality of life.

The term “cancer” refers to or describes the physiological condition in mammals that is typically characterized by unregulated cell growth. The cancer amendable for treatment by the present invention includes, but is not limited to: carcinoma, lymphoma, blastoma, sarcoma, and leukemia or lymphoid malignancies. More particular examples of such cancers include squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high-grade immunoblastic NHL; high-grade lymphoblastic NHL; high-grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome. According to some embodiments, the cancer is selected from the group consisting of: non-small cell lung carcinoma (NSCLC), renal cell carcinoma, melanoma, glioblastoma, colorectal cancer, breast cancer and ovarian carcinoma. The cancerous conditions amendable for treatment of the invention include metastatic cancers.

The pharmaceutical composition according to the present invention may be administered together with or in combination with an anti-cancer composition.

As used herein the term “combination” or “combination treatment” can refer either to concurrent administration of the articles to be combined or sequential administration of the articles to be combined. As described herein, when the combination refers to sequential administration of the articles, the articles can be administered in any temporal order.

The term “treatment” as used herein refers to both therapeutic treatment and prophylactic or preventative measures. Those in need of treatment include those already with the disorder as well as those in which the disorder is to be prevented.

According to some embodiments, the method of treating cancer comprises administering the pharmaceutical composition as part of a treatment regimen comprising administration of at least one additional anti-cancer agent or treatment.

According to some embodiments, the anti-cancer agent is selected from the group consisting of an antimetabolite, a mitotic inhibitor, a taxane, a topoisomerase inhibitor, a topoisomerase II inhibitor, an asparaginase, an alkylating agent, an anti-tumor antibiotic, an immune-modulator, a checkpoint inhibitor, an antibody targeting a tumor antigen, and combinations thereof. Each possibility represents a separate embodiment of the invention.

According to some embodiments, the anti-metabolite is selected from the group consisting of cytarabine, fludarabine, fluorouracil, mercaptopurine, methotrexate, thioguanine, gemcitabine, and hydroxyurea. According to some embodiments, the mitotic inhibitor is selected from the group consisting of vincristine, vinblastine, and vinorelbine. According to some embodiments, the topoisomerase inhibitor is selected from the group consisting of topotecan and irinotecan. According to some embodiments, the alkylating agent is selected from the group consisting of busulfan, carmustine, lomustine, chlorambucil, cyclophosphamide, cisplatin, carboplatin, ifosfamide, mechlorethamine, melphalan, thiotepa, dacarbazine, and procarbazine. According to some embodiments, the anti-tumor antibiotic is selected from the group consisting of bleomycin, dactinomycin, daunorubicin, doxorubicin, idarubicin, mitomycin, mitoxantrone, and plicamycin. According to some embodiments, the topoisomerase II is selected from the group consisting of etoposide and teniposide. Each possibility represents a separate embodiment of the present invention.

According to some particular embodiments, the additional anti-cancer agent is selected from the group consisting of bevacizumab, carboplatin, cyclophosphamide, doxorubicin hydrochloride, gemcitabine hydrochloride, topotecan hydrochloride, thiotepa, and combinations thereof. Each possibility represents a separate embodiment of the present invention.

Antibodies according to the present invention may be used as part of combined therapy with at least immuno-modulator, an activated lymphocyte cell, a kinase inhibitor or a chemotherapeutic agent.

According to some embodiments, the anti-cancer agent is an immuno-modulator, whether agonist or antagonist, such as antibody against an immune checkpoint inhibitor.

Checkpoint immunotherapy blockade has proven to be an exciting new venue of cancer treatment. Immune checkpoint pathways consist of a range of co-stimulatory and inhibitory molecules which work in concert in order to maintain self-tolerance and protect tissues from damage by the immune system under physiological conditions. Tumors take advantage of certain checkpoint pathways in order to evade the immune system. Therefore, the inhibition of such pathways has emerged as a promising anti-cancer treatment strategy.

The anti-cytotoxic T lymphocyte 4 (CTLA-4) antibody ipilimumab (approved in 2011) was the first immunotherapeutic agent that showed a benefit for the treatment of cancer patients. The antibody interferes with inhibitory signals during antigen presentation to T cells. Anti-programmed cell death 1 (PD-1) antibody pembrolizumab (approved in 2014) blocks negative immune regulatory signaling of the PD-1 receptor expressed by T cells. An additional anti-PD-1 agent was filed for regulatory approval in 2014 for the treatment of non-small cell lung cancer (NSCLC). Active research is currently exploring many other immune checkpoints, among them: CEACAM1, NKG2A, B7-H3, B7-H4, VISTA, lymphocyte activation gene 3 (LAG3), CD137, OX40 (also referred to as CD134), and killer cell immunoglobulin-like receptors (KIR).

According to other embodiments the additional anti-cancer agent is a chemotherapeutic agent. The chemotherapy agent, which could be administered together with the antibody according to the present invention, or separately, may comprise any such agent known in the art exhibiting anticancer activity, including but not limited to: mitoxantrone, topoisomerase inhibitors, spindle poison from vinca: vinblastine, vincristine, vinorelbine (taxol), paclitaxel, docetaxel; alkylating agents: mechlorethamine, chlorambucil, cyclophosphamide, melphalan, ifosfamide; methotrexate; 6-mercaptopurine; 5-fluorouracil, cytarabine, gemcitabine; podophyllotoxins: etoposide, irinotecan, topotecan, dacarbazine; antibiotics: doxorubicin (adriamycin), bleomycin, mitomycin; nitrosoureas: carmustine (BCNU), lomustine, epirubicin, idarubicin, daunorubicin; inorganic ions: cisplatin, carboplatin; interferon, asparaginase; hormones: tamoxifen, leuprolide, flutamide, and megestrol acetate.

According to some embodiments, the chemotherapeutic agent is selected from alkylating agents, antimetabolites, folic acid analogs, pyrimidine analogs, purine analogs and related inhibitors, vinca alkaloids, epipodophyllotoxins, antibiotics, L-asparaginase, topoisomerase inhibitor, interferons, platinum coordination complexes, anthracenedione substituted urea, methyl hydrazine derivatives, adrenocortical suppressant, adrenocorticosteroids, progestins, estrogens, antiestrogen, androgens, antiandrogen, and gonadotropin-releasing hormone analog. According to another embodiment, the chemotherapeutic agent is selected from the group consisting of 5-fluorouracil (5-FU), leucovorin (LV), irinotecan, oxaliplatin, capecitabine, paclitaxel and docetaxel. One or more chemotherapeutic agents can be used.

According to still another aspect the present invention provides a method of treating cancer in a subject in need thereof comprising administering to said subject a therapeutically effective amount of an antibody or antibody conjugate according to the present invention.

Toxicity and therapeutic efficacy of the compositions described herein can be determined by standard pharmaceutical procedures in cell cultures or experimental animals, e.g., by determining the IC50 (the concentration which provides 50% inhibition) and the maximal tolerated dose for a subject compound. The data obtained from these cell culture assays, and animal studies can be used in formulating a range of dosages for use in humans. The dosage may vary depending inter alia upon the dosage form employed, the dosing regimen chosen, the composition of the agents used for the treatment and the route of administration utilized, among other relevant factors. The exact formulation, route of administration and dosage can be chosen by the individual physician in view of the patient's condition. Depending on the severity and responsiveness of the condition to be treated, dosing can also be a single administration of a slow-release composition, with course of treatment lasting from several days to several weeks or until cure is affected or diminution of the disease state is achieved. The amount of a composition to be administered will, of course, be dependent on the subject being treated, the severity of the affliction, the manner of administration, the judgment of the prescribing physician, and all other relevant factors.

The term “administering” or “administration of” a substance, a compound or an agent to a subject can be carried out using one of a variety of methods known to those skilled in the art. For example, a compound or an agent can be administered enterally or parenterally. Enterally refers to administration via the gastrointestinal tract including per os, sublingually or rectally. Parenteral administration includes administration intravenously, intradermally, intramuscularly, intraperitoneally, subcutaneously, ocularly, sublingually, intranasally, by inhalation, intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods. In some embodiments, the administration includes both direct administrations, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient.

Antibodies are generally administered in the range of about 0.1 to about 50 mg/kg of patient weight, commonly about 0.5 to about 20 mg/kg, and often about 1 to about 10 mg/kg. In this regard, it is preferred to use antibodies having a circulating half-life of at least 12 hours, preferably at least 4 days, more preferably up to 21 days. In some cases, it may be advantageous to administer a large loading dose followed by periodic (e.g., weekly) maintenance doses over the treatment period. Antibodies can also be delivered by slow-release delivery systems, pumps, and other known delivery systems for continuous infusion.

The term “about” means that an acceptable error range, e.g., up to 5% or 10%, for the particular value should be assumed.

The terms “a,” “an,” and “the” are used herein interchangeably and mean one or more.

The term “and/or” is used to indicate one or both stated cases may occur, for example A and/or B includes, (A and B) and (A or B).

The term “or,” as used herein, denotes alternatives that may, where appropriate, be combined; that is, the term “or” includes each listed alternative separately as well as their combination if the combination is not mutually exclusive.

The terms “comprising”, “comprise(s)”, “include(s)”, “having”, “has” and “contain(s),” are used herein interchangeably and have the meaning of “consisting at least in part of”. When interpreting each statement in this specification that includes the term “comprising”, features other than that or those prefaced by the term may also be present. Related terms such as “comprise” and “comprises” are to be interpreted in the same manner. The terms “have”, “has”, having” and “comprising” may also encompass the meaning of “consisting of” and “consisting essentially of”, and may be substituted by these terms. The term “consisting of” excludes any component, step or procedure not specifically delineated or listed. The term “consisting essentially of” means that the composition or component may include additional ingredients, but only if the additional ingredients do not materially alter the basic and novel characteristics of the claimed compositions or methods.

The following methods and examples are presented in order to more fully illustrate some embodiments of the invention. They should, in no way be construed as limiting the scope of the invention.

Generally, the nomenclature used herein, and the laboratory procedures utilized in the present invention include molecular, biochemical, microbiological, immunological and recombinant DNA techniques. Such techniques are well known in the art. Other general references referring to well-known procedures are provided throughout this document for the convenience of the reader.

### Materials and Methods

### ELISA Assay

Binding specificity and affinity of antibody variants were determined by ELISA using recombinant mouse or human GITR (SinoBiological). ELISA plates (Nunc) were coated overnight at 4° C. with the indicated recombinant GITR (1 μg/mL/well). All sequential steps were performed at room temperature in phosphate buffer saline (PBS) supplemented with 2% Bovine serum Albumin (BSA). After being washed, the plates were blocked for 1 hr and were subsequently incubated for 1 hr with serially diluted antibody samples. After washing, plates were incubated for 1 hr with HRP-conjugated anti-mouse IgG (Jackson ImmunoResearch). Detection was performed by TMB Soluble Reagent (3,3′,5,5′-Tetramethylbenzidine, ScyTek Laboratories) and reactions stopped with the addition of 0.18 M sulfuric acid. Absorbance at 450 nm was immediately recorded using a SpectraMax Plus spectrophotometer (Molecular Devices) and background absorbance from negative control samples was subtracted.

The following modifications of the protocol described above were performed for FcγR binding ELISA. Human FcγRs soluble ectodomains (2 μg/ml/well) were immobilized to the plate. After being washed, the plates were blocked for 40 minutes with PBS with 10% BSA and were subsequently incubated for 2 hours with serially diluted antibodies.

### In Vivo Studies in Mice

C57BL/6J mice were purchased from Harlan Laboratories. FcγR humanized mice (mFcγRa, Fcgr1^{-/-}, hFcγRI⁺, hFcγRIIAR¹³¹⁺, hFcγRIIB⁺, hFcγRIIIAF¹⁵⁸⁺, and hFcγRIIIB⁺) was generated in the C57Bl/6J background as described in Rankin C T, et al., (CD32B, the human inhibitory Fc-gamma receptor IIB, as a target for monoclonal antibody therapy of B-cell lymphoma. Blood, 2006, 108, 2384-91).

To assess the modulation of the immune cells following administration of anti hGITR Fc region variants, mice humanized for FcγR and hGITR (huFcγR/hGITR) were generated.

For the pharmacokinetics assay assessing the effect of the Fc region modification on in-vivo half-life of the antibodies, MC38 (mouse colon adenocarcinoma) tumor-bearing human FcγR mice were injected with hIgG1 Fc region variants of DTA-1 anti mouse GITR (200 μg/mouse) and bled at indicated time points.

For assessing anti-tumor activity (with one challenge or first challenge), FcγR humanized mice inoculated with MC38 cells were treated with chimeric human Fc region variants based on the Fab of the anti mGITR antibody DTA-1. Mice, 8-10 weeks of age, were anesthetized and implanted subcutaneously (s.c) with MC38 cells (2×10⁶) in the right flank. Tumor volumes were measured every 2-3 days with an electronic caliper and reported as volume using the formula (L2²×L1)/2, where L1 is the longest diameter and L2 is the shortest diameter. After tumor inoculation, mice were randomized by tumor size (day 0), average of 55 mm³ or 115 mm³ (unless otherwise indicated) and received intraperitoneal (i.p) injection of the Abs, or PBS. Mice were treated with 100 μg/mouse of antibody variant comprising hIgG1 and an additional antibody treatment at days 3 and 6, as indicated.

For the re-challenge experiment assessing long term immunity and memory response, mice with complete response and rejection of the primary tumor (tumor-free mice) were followed for 62 days after the treatment onset and then were re-inoculated with MC38 tumor cells (2×10⁶) on the contrary flank.

For the CD4/CD8 depletion experiment, several tumor-free mice were injected with antibodies against CD4 and/or CD8 prior to re-challenge.

### Tissue Processing and Flow Cytometry

For functional experiments, mice were challenged and treated as described above, and were killed at day 8, unless otherwise indicated. Spleens were dissected through a 70 μm nylon cell strainer, incubated with red blood cells lysis buffer (Sigma), and washed. Tumors were mechanically dissected into small pieces and transferred to GentleMACS™ C tube (Milteny) with 0.33 mg/ml DNase (Sigma-Aldrich) and 0.27 mg/ml Liberase TL (Roche). GentleMACS™ Octo Dissociator program “m_impTumor_02_01” (Milteny) was performed twice before incubating for 40 minutes at 37° C. with continuous rotation at 25 rpm. Before dispersed through a 70 μm nylon cell strainer and washed, program “m_impTumor_03_01” (Milteny) was performed twice. Lymph nodes were dissected through a 70 μm nylon cell strainer and washed.

Different cell populations were identified after excluding dead cells using live/dead fixable blue dead cell satin kit (Invitrogen). For intracellular staining, cells were fixed and permeabilized with Foxp3 Fix/Perm buffer kit (BioLeagend). CountBright™ Absolute Counting Beads (Life Technologies) were added prior to acquisition. Cell populations were defined by the following markers (BioLegened dendritic cells (CD11b⁺ CD11c⁺ MHCII⁺ F4/80⁻), NK cells (NKp46⁺ CD3⁻), CD8 T cells (CD3⁺ CD8⁺ CD4⁻), CD4 T cells (CD3⁺ CD4⁺ CD8⁻), CD4 effector T cells (CD3⁺ CD4⁺ CD8⁻ Foxp3⁻ CD44⁺), and CD4 regulatory T cells (CD3⁺ CD4⁺ CD8⁻ Foxp3⁺).

### Mass Spectrometry

Samples were digested with trypsin using the S-trap method, followed by HILIC enrichment of glycopeptides. The resulting peptides were analyzed using nanoflow liquid chromatography (nanoAcquity) coupled to high resolution, high mass accuracy mass spectrometry (Fusion Lumos). The resulting data was searched using Byonic, against the human IgG1 glycosylated peptide and the human Fc glycan database. ID's were verified manually, and quantified using Skyline (v19.1.0.193). The experiment was performed and analyzed by the INCPM unit in Weizmann Institute of Science.

### Determination of Dissociation Constant (K_{D}) by Surface Plasmon Resonance (SPR)

SPR experiments were performed using a Biacore™ T200 (Cytiva) instrument. Antibodies were captured onto a Protein G Chip. All measurements were done using PBS-Tween 0.05%. Antibodies at a concentration of 5 μg/mL were immobilized for 20s at a flow rate of 10 L/min. FcγRs were prepared in different concentration ranges: 0.78 nM-200 nM FcγRIIA, 2.34-600 nM FcγRIIIA and 2-500 nM FcγRIIB. Each concentration was injected for 180s at a flowrate of 30 μL/min and left to dissociate for 340s. After each cycle, the surface was regenerated with Glycine Buffer pH 1.5. Background binding to the blank immobilized path was subtracted from each binding event. Data were fit to a Two State 1:1 binding model using the T200 Evaluation Software and K_{D} values were calculated. Calculation of the activating to inhibitory affinity ratio of every variant was done as follows: K_{D} activating/K_{D} inhibitory.

### Statistical Analysis

An unpaired two-tailed t test was used when two groups were compared and to compare groups in experiments assessing percentages of cell types. Data were analyzed with GraphPad Prism software (GraphPad) and p values of <0.05 were considered statistically significant, indicated as *p≤5 0.05, **p≤5 0.01, and ***p≤0.001 in the figures. Asterisks indicate statistical comparison as indicated on the graphs.

### EXAMPLES

Reference is now made to the following examples, which together with the above descriptions, illustrate the invention in a non-limiting fashion.

### Example 1. Generation and Characterization of Anti GITR hIgG1 Fc Region Variants

To generate chimeric anti mouse GITR antibodies having human IgG1 from the rat anti-mouse DTA-1 clone, the variable regions of the heavy and light chains were sequenced from DTA-1 hybridoma clone and synthesized (by BioBasic). To generate humanized anti-human GITR antibodies, the heavy chain (HC) and the light chain (LC) sequences of the anti hGITR antibody were used and synthesized (by BioBasic). Sequences were PCR-amplified and cloned into mammalian expression vectors with human IgG1 constant regions. FIG. 2, present gel electrophoresis results of the HC and LC expression vectors following the insertion of anti-GITR VH and VL. FIG. 2, present gel electrophoresis results of the HC and LC expression vectors following the insertion of anti-GITR VH and VL from TRX518 (This gel is DTA-1). Lane 1—1 Kb molecular weight marker (Mw), lane 2—100 bp Mw, lane 3-uncut expression vector, lane 4 and 5—vectors with an anti-GITR VH insert, lane 6—vector with an anti-GITR VL and LC constant region (Fc), Lane 7—HC IgG1 expression vector with the constant region (Fc), lane 8—HC IgG1 expression vector with the constant region (Fc) with Vii mutations in the constant heavy region. lane 9—LC IgG1 expression vector. For the generation of Fc-modified variants of human IgG1, site-directed mutagenesis techniques were applied to create pre-defined point mutations by PCR using the specific primers listed in Table 1 (Agilent Technologies) according to the manufacturer's instructions.

TABLE 1
Primers used for generation of Fc-
modified variants

| | | Primer sequence | SEQ |
|---|---|---|---|
| Variant | Substitution | 5′->3′ | ID NO: |
| G236A | G236A | GCACCTGAACTCCTG | 16 |
| GAALIE | | gccGGACCGTCAGTC | |
| | | TTC | |
| GASDALIE | G236A/S239D | GCACCTGAACTCCTG | 17 |
| | | GccGGACCGgacGTC | |
| | | TTCCTCTTCCC | |
| GASDALIE | A330L/1332E | CAACAAAGCCCTCCC | 18 |
| GAALIE | | ActgCCCgagGAGAA | |
| | | AACCATCTCC | |
| N297A | N297A | CGGGAGGAGCAGTAC | 19 |
| | | gcCAGCACGTACCGT | |
| | | GTG | |
| V11 | G237D/P238D | CTGAACTCCTGGGGG | 20 |
| | | acgacTCAGTCTTCC | |
| | | TCTTC | |
| | H268D/P271G | GTGGTGGACGTGAGC | 21 |
| | | gACGAAGACggTGAG | |
| | | GTCAAGTTCAAC | |
| | A330R | CAACAAAGCCCTCCC | 30 |
| | | AcgCCCCATCGAGAA | |
| | | AAC | |

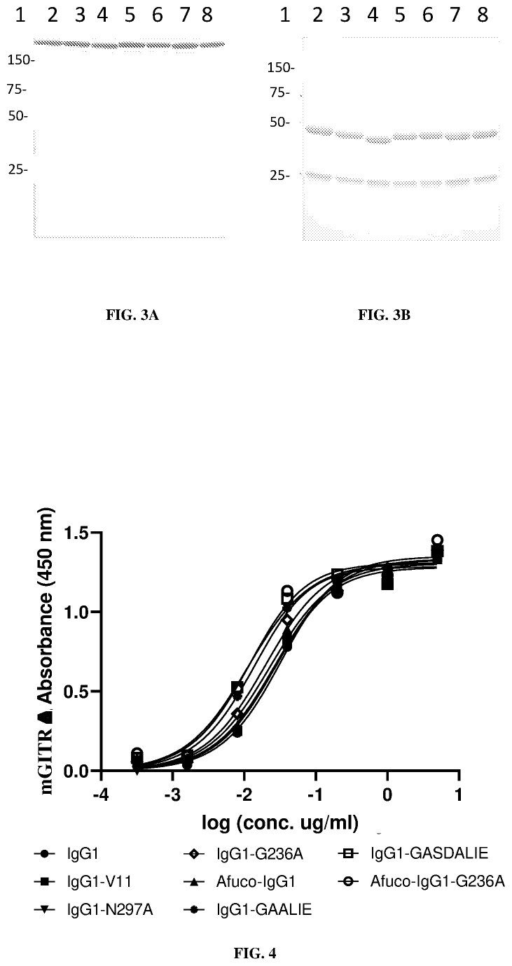

Mutated plasmid sequences were validated by direct sequencing (Life science core facility, Weizmann Institute of Science). To produce antibodies, heavy and light chain expression vectors were transfected transiently into Expi293 cells (ThermoFisher). The antibodies secreted to the supernatant were purified by protein G Sepharose 4 Fast Flow (GE Healthcare). Purified antibodies were dialyzed in PBS and sterile filtered (0.22 μm) and the purity was assessed by SDS-PAGE followed by Imperial™ blue staining (ThermoFisher), which demonstrated that all antibodies were expressed in expected form. For creation of afucosylated Fc region variants, 200 μM of 2-Deoxy-2-fluoro-L-fucose (Carbosynth), as an inhibitor of fucosyltransferase, was added to the transfection medium, one-day post transfection to the supernatant. FIG. 3A-3B present SDS page stained with Imperial™ blue of the expressed anti-GITR variants: lane 1—molecular weight marker, lane 2—anti-mGITR V11, lane 3—afucosylated anti-mGITR, lane 4—N279A, lane 5—GAALIE, lane 6—GASDALIE, lane 7—G236A, lane 8 anti-mGITR IgG1. FIG. 3A, presents results of a non-reduced SDS-PAGE. FIG. 3B, presents results of a reduced SDS-PAGE.

To test whether the activity of anti mouse GITR (mGITR) chimeric antibodies comprising human IgG1 requires interaction with hFcγR and to determine whether such interactions can be further engineered to optimize the activity of the parent antibody, the variable regions of the anti mouse GITR (DTA-1) antibody was cloned into human IgG1. Different mutations were introduced, by site-directed mutagenesis, into the CH2 domain of the Fc region to generate a series of mutated hIgG1 anti-mGITR antibodies. Binding affinities were measured using SPR analysis with immobilized FcγRs and soluble antibodies and the results are indicated in Table 2.

TABLE 2
Binding affinities of IgG Fc region variants for selected human FcγRs.

| | Inhibitory | | Activating | | | |
|---|---|---|---|---|---|---|
| | huFcγRIIB | | huFcγRIIA^{R131} | | huFcγRIIIA^{F158} | |
| IgG1 | K_{D}(μM) | Fold* | K_{D}(μM) | Fold | K_{D}(μM) | Fold |
| Wildtype | 3.01 | 1 | 1.16 | 1 | 6.7 | 1 |
| N297A | n.b | | n.b | | n.b | |
| G236A | 4.38 | 0.7 | 0.12 | 9.3 | 3.9 | 1.7 |
| G236A/S239D/A330L/I332E | 0.23 | 14 | 0.05 | 24.5 | 2.23 | 29.5 |
| Afucosylated-IgG1 | 2.8 | 1.1 | 1.16 | 1 | 0.58 | 11.6 |
| G237D/P238D/H268D/P271G/ | 0.03 | 95.6 | 3.84 | 0.3 | n.b | |
| A330R (V11) | | | | | | |

*Fold = K_{D}(IgG1)/K_{D}(Fc region variant).
n.b = no binding.

Following production, the binding affinities of the Fc region variants to mouse GITR were measured and verified by comparative ELISA. FIG. 4 present results of ELISA assay for the Fc region variants to mouse GITR; IgG1 (full circle), IgG1-V11 (full square), IgG1-N297A (upside down triangle), IgG1-G236A (rhombus), Afuco-IgG1 (triangle), IgG-GAALIE (asterisk), IgG1-GASDALIE (empty squares), and Afuco-IgG1-G236A. As shown in FIG. 4, chimeric anti mGITR (comprising the Fab of DTA-1) hIgG1 Fc region variants show binding affinity similar to the parent antibody, indicating that the Fc region modification does not impair binding to mouse GITR.

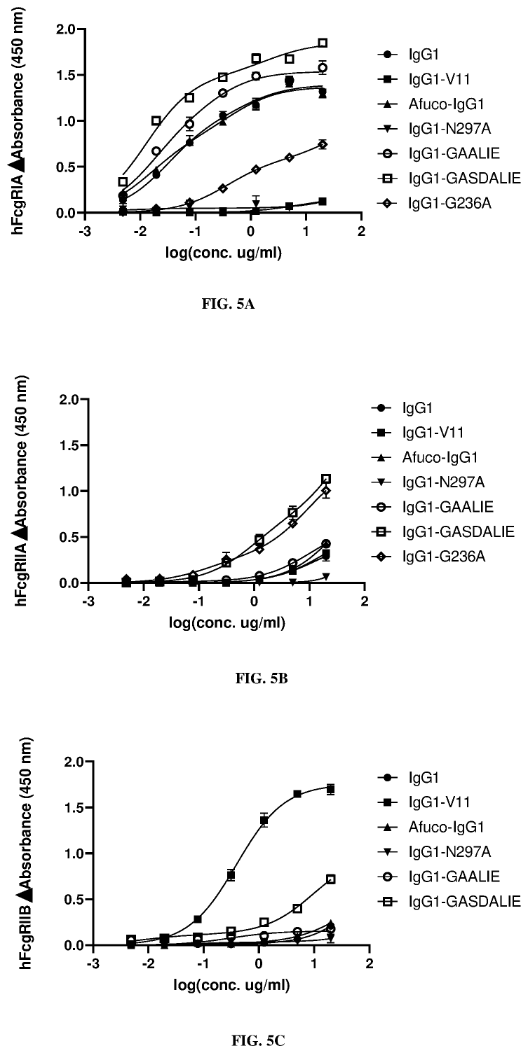

Further, the binding affinities of anti mGITR antibodies having modified human IgG1, to each FcγR were compared and evaluated using comparative ELISA. FIG. 5A-5D present the comparative EISA results for the binding of the anti-mGITR Fc region variants: IgG1 (full circle), IgG1-V11 (full square), Afuco-IgG1 (triangle), IgG1-N297A (upside down triangle), IgG-GAALIE (empty circle), IgG1-GASDALIE (empty squares), and IgG1-G236A (empty rhombus). Anti mouse GITR antibodies having DTA-1 binding site and modified human IgG1 demonstrate differential binding affinities to the indicated hFcγRs. The human IgG1 variant with the multiple Fc region mutations G236A/S239D/A330L/I332E (termed “GASDALIE”), mainly enhances hFcRγIIIA binding, but also binds hFcγRIIA and hFcγRIIB. Similar variant without the S239D mutation (G236A/A330L/I332E, termed “GAALIE”), losses the binding to hFcγRIIB, while preserving the enhanced binding to hFcRγIIIA and hFcRγIIA. The variant “IgG1-G236A” shows selective enhancement of hFcγRIIA binding, and the “IgG1-V11” variant (G237D/P238D/H268D/P271G/A330R, GDPDHDPGAR) selectively enhances hFcγRIIB and exhibits no binding to human FcγRIIIA. The aglycosylated FcγR-null variant “IgG1-N297A” lacks the ability to engage hFcγR pathways due to modification of the glycosylation site. The post-translationally glycoengineered IgG1 variant “Afuco-IgG1”, that comprises non modified Fc-N297 enhances the binding to hFcγRIIIA.

Two non-fucosylated forms of anti GITR variants, namely IgG1 and IgG1-G236A were produced using fucosyltransferases inhibitor (2FF) which was added one-day post transfection to the transfection medium of IgG1 and IgG1-G236A producing cells. Fucosylation was determined by mass spectrometry and percentages of non-fucosylated forms are presented in Table 3. Antibody concentration was measured using NanoDrop machine.

TABLE 3
Percentages of non-fucosylated forms of the anti-GITR antibodies.

| | | Change fold |
|---|---|---|
| GITR variants | % of non-fucosylated forms | (relative to IgG1) |
| IgG1 | ~0.6% | |
| Afuco-IgG1 | ~74.2% | ~123 |
| Afuco-IgG1-G236A | ~80.6% | ~134 |

### Example 2. Pharmacokinetics of Anti mGITR IgG1 Fc Region Variants

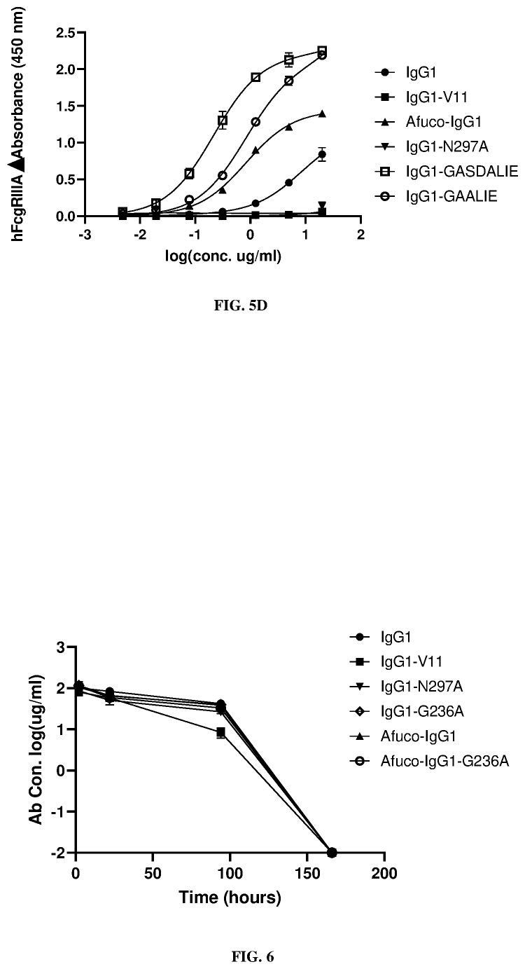

The effect of the Fc region modification on in-vivo half-life of the antibody was tested. MC38 tumor-bearing human FcγR mice were injected with hIgG1 Fc region variants of anti mGITR comprising the Fab of DTA-1 (200 μg/mouse) and bled at indicated time points. Serum was stored at −80 C until all time points were collected. Antibody concentration in serum was determined using a standard colorimetric ELISA assay. Briefly, assay plates were coated with recombinant mouse GITR (1 μg/mL, Sino Biological, #13643-H08H) and incubated overnight at 4° C. Plates were then blocked for 2 hours with PBS containing 10% FCS. Serial dilutions of serum were added to the plates and incubated for 2 hours. After washing, plates were incubated for 1 hour with horseradish peroxidase conjugated anti human IgG (#109-035-088, Jackson IummunoResearch). Absorbance at 450 nm was immediately recorded using a SpectraMax Plus spectrophotometer (Molecular Devices), and background absorbance from negative control samples was subtracted. FIG. 6 presents ELISA results of in-vivo half-life (PK assay) of the Fc-modified variants, IgG1 (full circle), IgG1-V11 (full square), IgG1-N297A (upside down triangle), IgGa-G236A (empty rhombus), Afuco-IgG1 (triangle), and, Afuco-IgG1-G236A (empty circle).

As indicated in FIG. 6, the in-vivo half-life of the Fc-modified variants is similar to that of the antibody with parental IgG1, except for “IgG1-V11” which demonstrate decreased level in the blood 4 days post injection.

### Example 3. In-Vivo Anti-Tumor Activity of Anti-GITR hIgG1 Fc Region Variants

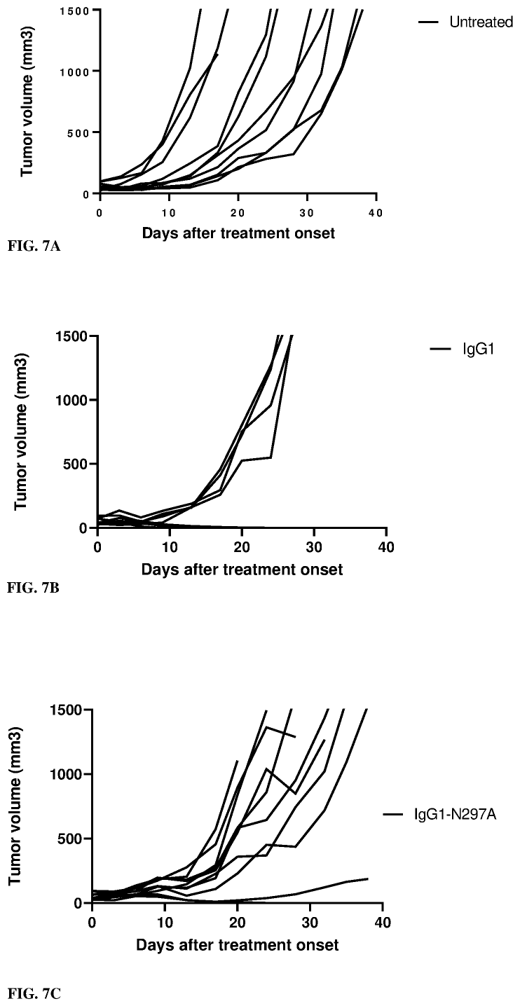

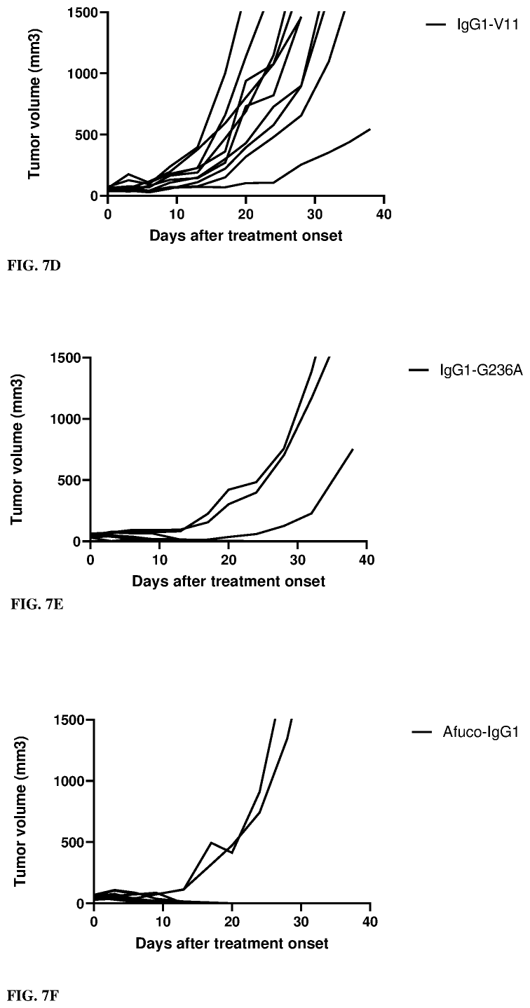

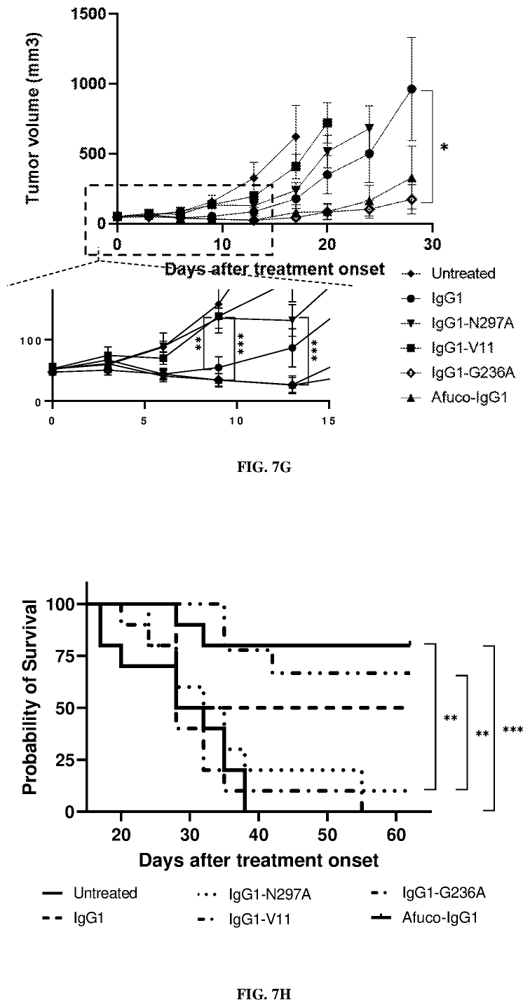

The anti-tumor activity of anti mGITR hIgG Fc region variants was evaluated in-vivo in FcγR humanized mice inoculated with MC38 (mouse colon adenocarcinoma) cells and treated with human Fc region variants of DTA-1. FcγR humanized C57BL/6J mice (mFcγRα^{-/-}, Fcgr1^{-/-}, hFcγRI⁺, hFcγRIIAR¹³¹⁺, hFcγRIIB⁺, hFcγRIIIAF¹⁵⁸⁺, and hFcγRIIIB⁺) were generated in the C57Bl/6J background as described in Rankin C T, et al., Blood, 2006 ibid. Mice, 8-10 weeks of age, were anesthetized and implanted subcutaneously (s.c) with MC38 cells (2×10⁶) in the right flank. Tumor volumes were measured every 2-3 days with an electronic caliper and reported as volume using the formula (L2²×L1)/2, where L1 is the longest diameter and L2 is the shortest diameter. After tumor inoculation, mice were randomized by tumor size (day 0), average of 55 mm³ (unless otherwise indicated), and received intraperitoneal (i.p) injection of the Abs, or PBS. For the anti-tumor activity, mice were treated with 100 μg/mouse anti mGITR (DTA-1-based) comprising hIgG and received an additional antibody treatment at days 3 and 6. For the re-challenged experiment, tumor-free mice from all treated groups were re-challenged with MC38 tumor cells (2×10⁶) in the contrary flank 62 days post anti GITR treatment onset. Mice were followed for tumor progression, and overall survival and the results are depicted in FIGS. 7A-7H.

Tumor volume and survival were evaluated for each tested animal of each human Fc region variant antibody and an unpaired two-tailed t test was used to compare between Fc variants to IgG1-N297A group. Each line represents one animal. The results were as follow: untreated mice (n=10), as expected no tumor free mice (FIG. 7A), IgG1 treatment (n=8), 4/8 tumor free mice (FIG. 7B), IgG1-N297A treatment (n=10), 1/10 tumor free mice (FIG. 7C), IgG1-V11 treatment (n=10), 0/10 tumor free mice (FIG. 7D), IgG1-G236A treatment (n=9), 6/9 tumor free mice (FIG. 7E), Afuco-IgG1 treatment (n=10), 8/10 tumor free mice (FIG. 7F). The average tumor volume of all tested animals per each Fc region variant is shown in FIG. 7G, Naïve (untreated, full rhombus), IgG1 (full circle), IgG1-N297A (upside down triangle), IgG1-G236A (empty rhombus), and Afuco-IgG1 (triangle). The probability of survival over time (up to 60 days post treatment onset) was also followed, results are presented in FIG. 7H, untreated mice (untreated, black line), IgG1 (dashed line), IgG1-N297A (dotted line), IgG1-V11 (dash and dot), IgG1-G236A dash and two dots) Afuco-IgG1 (line ending with a perpendicular segment). As indicated in FIG. 7H several treatments exhibited enhanced probability of survival, 80% for Afuco-IgG1, 66.6% for IgG1-G236A and 50% for IgG1. Treatment with Fc-engineered or glycoengineered variants IgG1-G236A and Afuco-IgG1 result in decreased tumor growth and increased overall survival compared to the other Fc region variants.

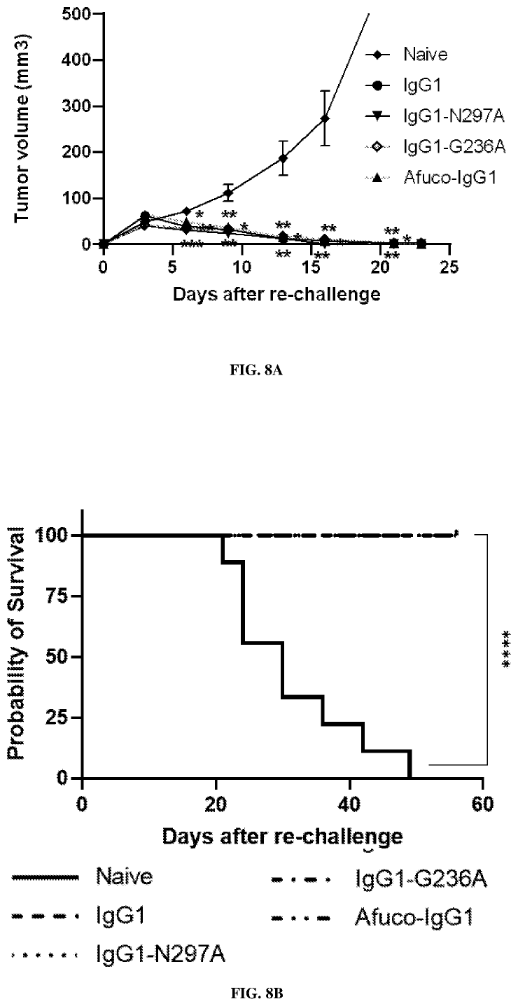

To evaluate the ability of anti-GITR treatments to mediate long-term immunity and memory response, tumor-free mice from all treated groups were re-challenged. Mice with complete response and rejection of the primary tumor were followed for 62 days after the treatment onset and then were re-inoculated on the contrary flank with 2×10⁶ MC38 tumor cells without any additional treatment. Mice were followed for tumor progression, and overall survival and the results are depicted as: tumor volume (FIG. 8A) over time (days after re-challenge) and probability of survival (FIG. 8B), calculated for each previous Fc region variant treatment (tumor-free animal). FIG. 8A Naïve (untreated, full rhombus, n=9), IgG1 (full circle, n=4), IgG1-N297A (upside down triangle, n=1), IgG1-G236A (empty rhombus, n=6), and Afuco-IgG1 (triangle, n=7). FIG. 8B Naïve (untreated, black line), IgG1 (dashed line), IgG1-N297A (dotted line), IgG1-G236A (dash and dot), and Afuco-IgG1 (dash and two dots). While tumors were rapidly developed in Naïve control mice, all animals which were previously treated with anti-GITR Abs, demonstrated long-term immunity, namely rejected the tumors and completely survived the re-challenge. GITR antibodies exhibit potential in mediating a long-term anti-tumor T cell memory protection against re-appearance of a tumor.

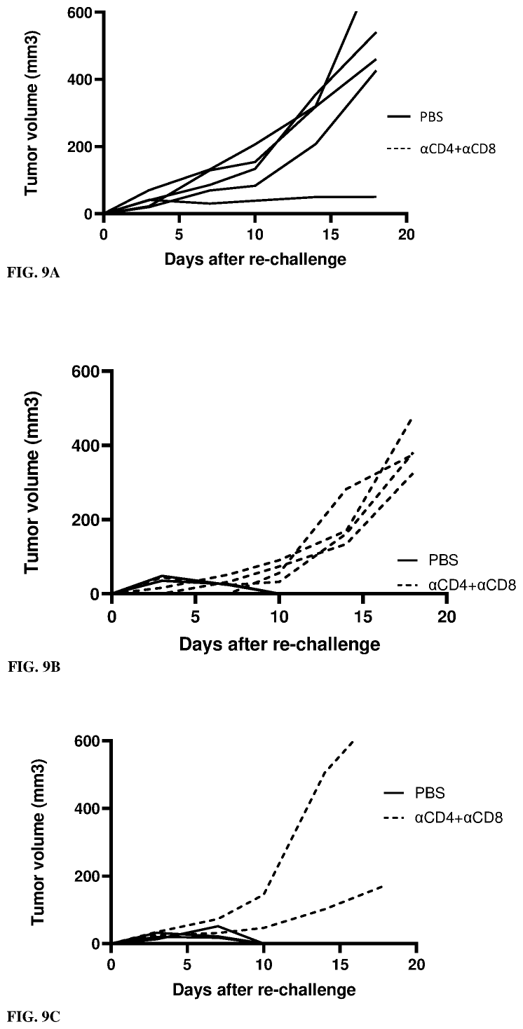

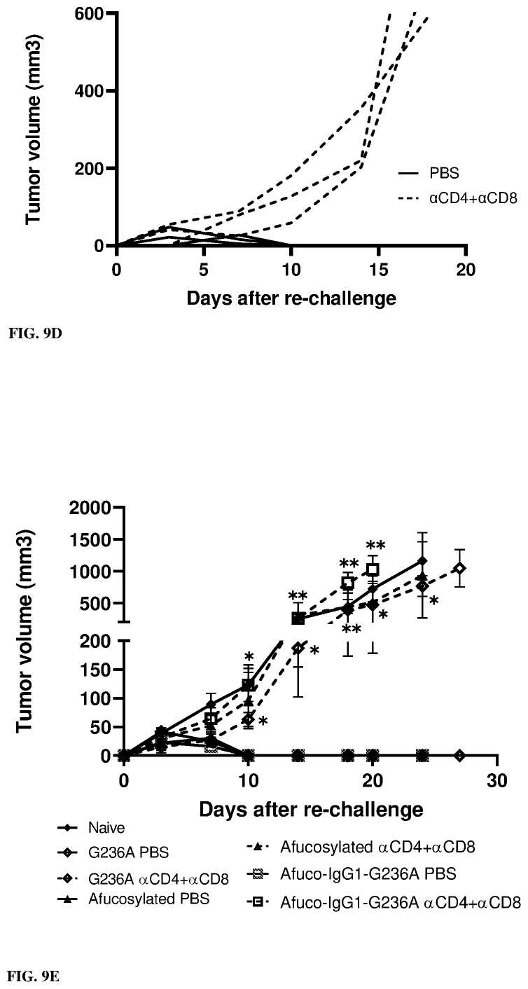

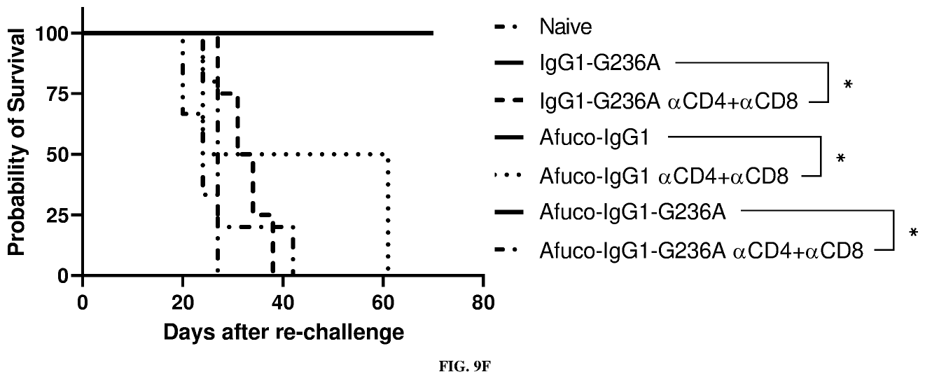

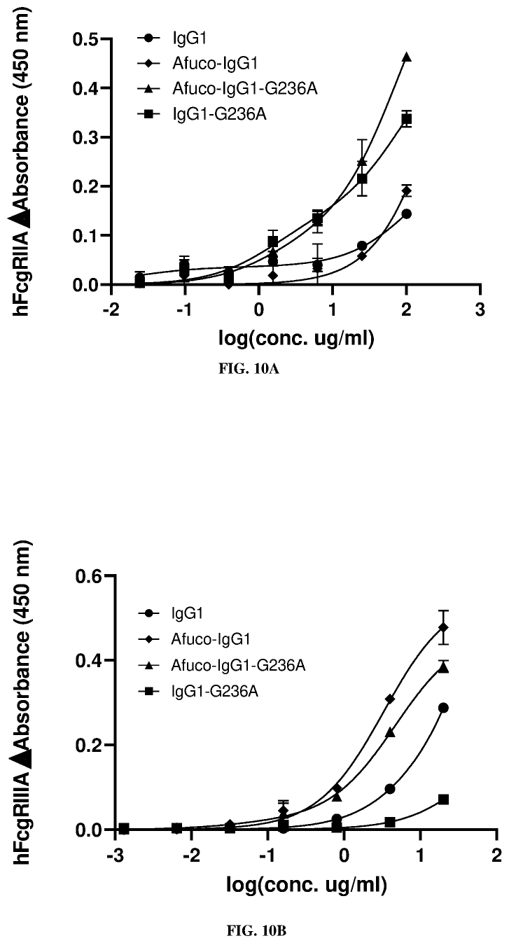

To further assess the mechanism of the long-term protection and memory response by GITR antibodies, the experiment was performed in a similar setting as above, but in addition, several tumor-free mice were injected with antibodies against CD4 or CD8. MC38-bearing hFcγR mice treated with anti-GITR Fc region variants that completely respond to previous treatment and become tumor free, and control Naïve mice, were inoculated with MC38 cells, injected with antibodies against CD4 and CD8, and followed for tumor growth. PBS was used as a control treatment. Tumor volume was followed over time for each animal tested. FIG. 9A-9D present the results for tumor growth progression in mice injected with aCD4 and aCD8 (dotted line) and with PBS as control (line) in addition to the treatment with the anti-GITR Fc region variants. FIG. 9A, control, untreated mice. FIG. 9B, IgG1-G236A. FIG. 9C, Afuco-IgG1. FIG. 9D, Afuco-IgG1-G236A. FIG. 9E present the average tumor volume for each previous Fc region variant treatment Naïve (n=6, full rhombus), IgG1-G236A with PBS (G236A PBS, n=2, empty rhombus and a black line), IgG1-G236A with aCD4 and aCD8 (G236A αCD4+αCD8, n=4, empty rhombus and a dashed line), Afuco-IgG1 with PBS (Afucosylated PBS, n=2, triangle and a black line), Afuco-IgG1 with aCD4 and aCD8 (Afucosylated αCD4+αCD8, n=3, triangle and a dashed line), Afuco-IgG1-G236A with PBS (n=3, empty squares and a black line), Afuco-IgG1-G236A with aCD4 and aCD8 (n=4, empty square and a dashed line). The probability of survival was calculated for each previous Fc region variant treatment presented in FIG. 9F, . Naïve (dash and two dots), IgG1-G236A (line), IgG1-G236A αCD4+αCD8 (dashed line), Afuco-IgG1 (line), Afuco-IgG1 with aCD4 and aCD8 (dotted line), Afuco-IgG1-G236A (line), and Afuco-IgG1-G236A with aCD4 and aCD8 (dash and dot). The abundance of CD8+ and CD4+ T cells in peripheral blood was determined by flow cytometry at various time points after antibody administration. For the flow cytometry analysis, fluorescently conjugated monoclonal antibodies targeting the β subunit of mouse CD8 (clone YTS156.7.7; Biolegend) or epitopes non-overlapping with the anti CD4 GK1.5 epitope (clone RM4-4; Thermofisher) were used to avoid competition with the depleting antibodies.

Taken together, the tumor growth and overall survival results assert a T cell-dependent mechanism for the long-term tumor immunity mediated by GITR antibodies. Tumor free mice were re-challenge with a second injection of tumor cells, then their T cell were depleted to test for T cell dependency on tumor reoccurrence. It was found that T cells are crucial for long-term immunity, because in their absence tumors expanded and were not rejected.

### Example 4. Afucosylated G236A Variant Improves the Antibody's Activation/Inhibitory Affinity Ratio

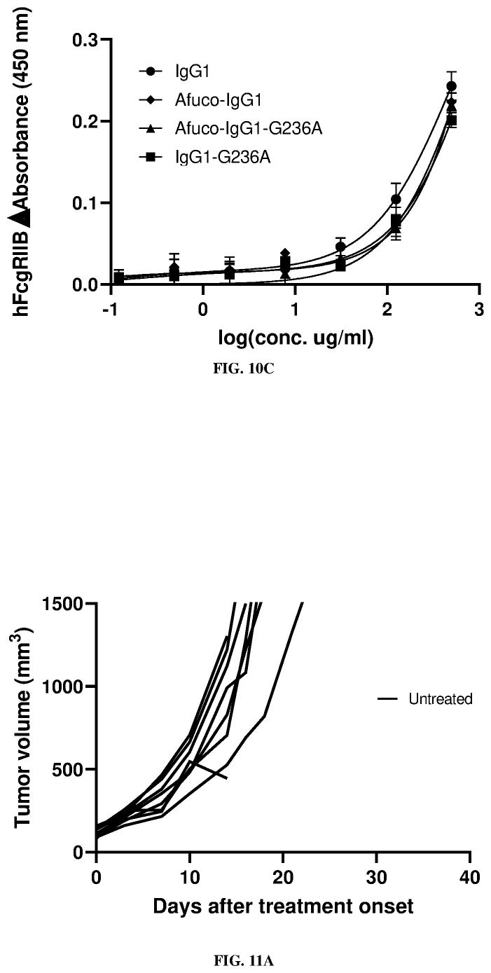

The activating receptor FcγRIIA is 90% homologous to the inhibitory receptor FcγRIIB (Rankin C. T. et al., Blood, 2006, ibid), therefore binding to the FcγRIIA and not to the FcγRIIB is a challenging task. To date, some modifications were introduced but most of them result with enhancement of binding to FcγRIIB (DAE, variant18 and GASDALIE disclosed in Smith P, et al., Mouse model recapitulating human Fcγ receptor structural and functional diversity. Proc Natl Acad Sci USA, 2012, 109, 6181-6). In the present invention, a new Fc region variant was created by combining two strategies to the Fc scaffold: glyco-engineering (afucosylation) and protein engineering by amino acid substitutions (G236A) to create the afucosylated IgG1-G236A variant. This double engineered antibody has enhanced binding to both FcγRIIA and FcγRIIIA activating receptors and subsequent capacity to elicit antibody-mediated effector functions, e.g., activation of dendritic cells and phagocytosis, while minimizing engagement to the inhibitory FcγRIIB. FIGS. 10A-10C presents ELISA assay results for the binding of the aGITR IgG1 (circle), Afuco-IgG1 (rhombus) Afuco-IgG1-G236A (triangle), and IgG1-G236A (square) to FcγRIIA (FIG. 10A), FcγRIIIA (FIG. 10B) and FcγRIIB (FIG. 10c).

The novel Afuco-IgG1-G236A variant that target GITR, has improved affinity toward both activating Fc receptors, relative to the other Abs tested, while having a seemingly lower affinity to the inhibitory FcγRIIB when compared to the hIgG1 (FIGS. 10A-10C).

### Example 5. FcγRIIA and FcγRIIIA Requirement for Optimal Anti mGITR Antibodies-Mediated Anti-Tumor Immunity

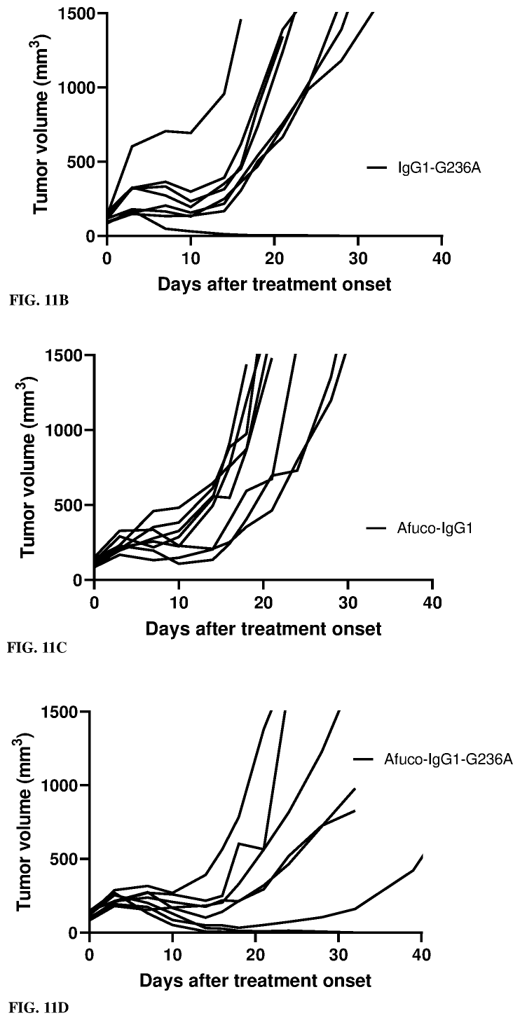

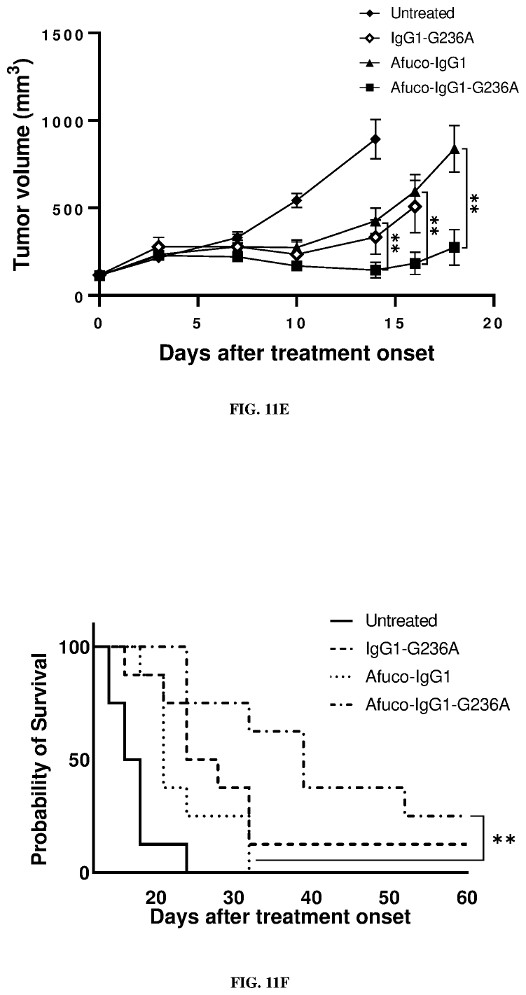

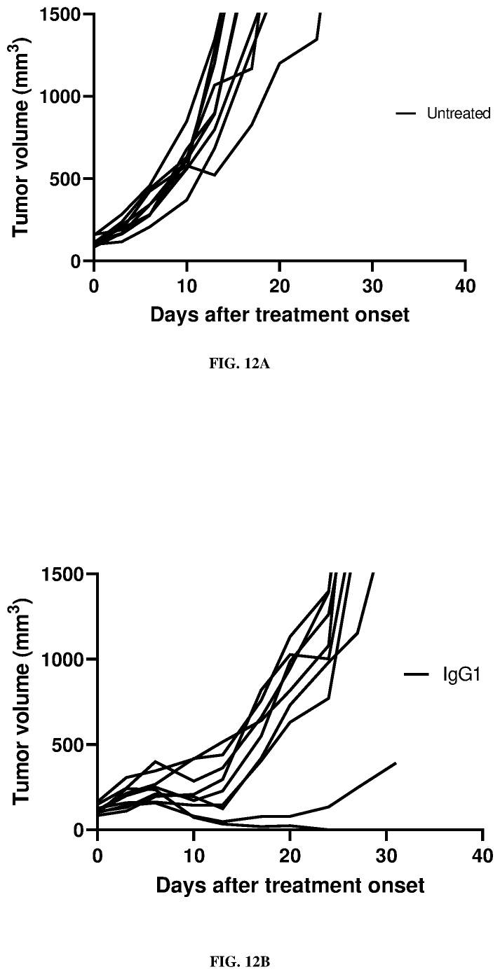

Since both afucosylated IgG1 and IgG1-G236A result with similar enhancement of anti-tumor activity, further evaluation was performed to establish whether one of these Fc variants is superior to the other and whether they engage distinct mechanisms (FcγRIIA vs. FcγRIIIA pathways). The anti-tumor activity was compared in vivo. Humanized FcγR mice were inoculated with MC38 cells and once tumor reached average volume of 115 mm³, the mice were treated with the indicated chimeric human Fc region variants (comprising the anti mGITR binding domain of DTA-1 antibody), IgG1-G236A, Afuco-IgG1 and Afuco-IgG1-G236A. Tumor progression was monitored for each animal (FIGS. 11A-11D), as average tumor volume for each treatment group (FIG. 11E), and as probability of survival (FIG. 11F). An unpaired two-tailed t test was used to compare between Afuco-IgG1-G236A and IgG1 groups (n=8 in all groups). The treatment groups were: untreated mice, no tumor free mice (FIG. 11A), IgG1-G236A treatment, 1/8 tumor free mice (FIG. 11B), Afuco-IgG1 treatment, 0/8 tumor free mice (FIG. 11C), and Afuco-IgG1-G236A treatment, 2/8 tumor free mice (FIG. 11D). FIG. 11E presents the average tumor volume for each treatment group, control, untreated mice (untreated, full rhombus), IgG-G236A (empty rhombus), Afuco-IgG1 (triangle), Afuco-IgG1-G236A (square). FIG. 11F presents the probability of survival of mice in each of the tested groups, untreated mice (untreated, line), IgG-G236A (dashed line), Afuco-IgG1 (dotted line), Afuco-IgG1-G236A (dash and dot).

As shown in the results, all three Fc region variants still result in significant anti-tumor activity. Mice treated with the Fc region variant Afuco-IgG1-G236A showing FcγRIIA and FcγRIIIA-enhancement, result with statistically significant decrease in tumor growth and complete response when comparing to Afuco-IgG1 but not to the IgG1-G236A variant.

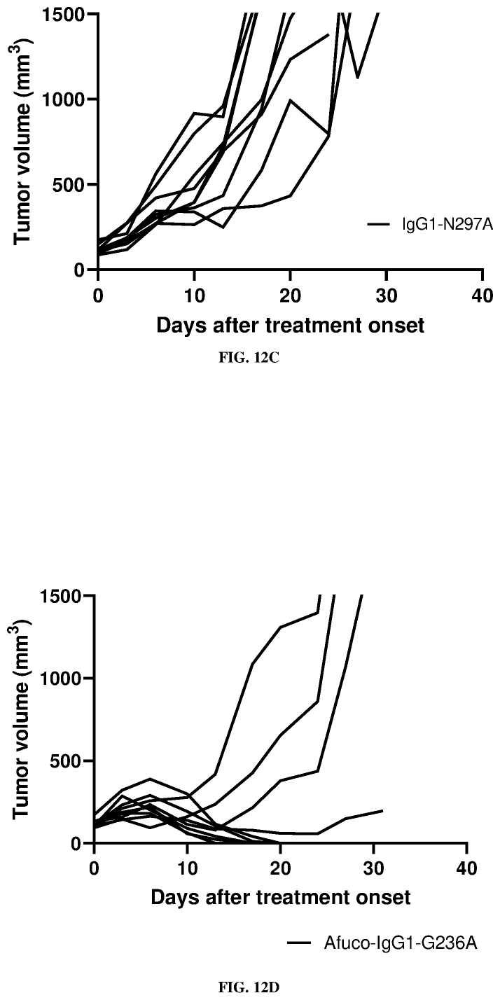

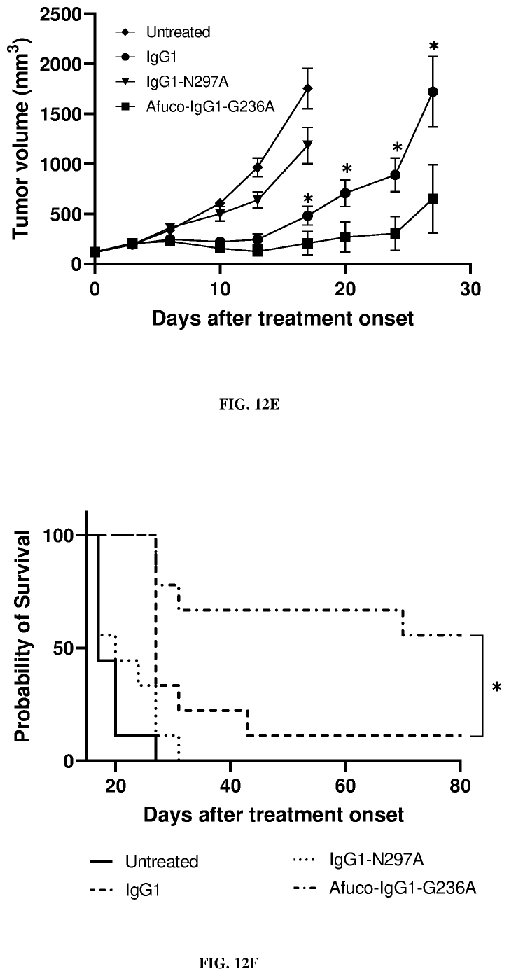

When comparing to IgG1 or IgG1-N297A, Fc scaffolds that are part of antibodies currently in clinical trials, the novel Afuco-IgG1-G236A demonstrates superior anti-tumor activity and overall survivor in advanced refractory tumor model. Humanized FcγR Mice were inoculated with MC38 cells and once tumor reached average volume of 115 mm³ they were treated with the indicated human Fc region variants, IgG1, IgG1-N297A, or Afuco-IgG1-G236A. Mice were followed for tumor progression (FIGS. 12A-12D), average tumor volume for each treatment group and overall probability of survival (FIG. 12E-12F). FIG. 12E presents the average tumor volume for each treatment group, control, untreated mice (untreated, full rhombus), IgG1 (circle), IgG1-N297A (upside down triangle), Afuco-IgG1-G236A (square). FIG. 12F presents the probability of survival of mice in each of the tested groups, untreated mice (untreated, line), IgG1 (dashed line), IgG1-N297A (dotted line), Afuco-IgG1-G236A (dash and dot).

As demonstrated in FIGS. 12E and 12F, the double modified-Fc region variant Afuco-IgG1-G236A decrease tumor growth and survival compared to the other Fc region variants. An unpaired two-tailed t test was used to compare between Afuco-IgG1-G236A to IgG1 group. The Fc region variant treatment groups are: untreated mice, no tumor free mice (FIG. 12A), IgG1 treatment, 1/9 tumor free mice (FIG. 12B), IgG1-N297A treatment, 0/9 tumor free mice (FIG. 12C), Afuco-IgG1-G236A treatment, 5/9 tumor free mice (FIG. 12D).

### Example 6. Impact of Anti mGITR hIgGs on the Tumor Microenvironment (TME) and Draining Lymph Node (dLN) Immune Landscape

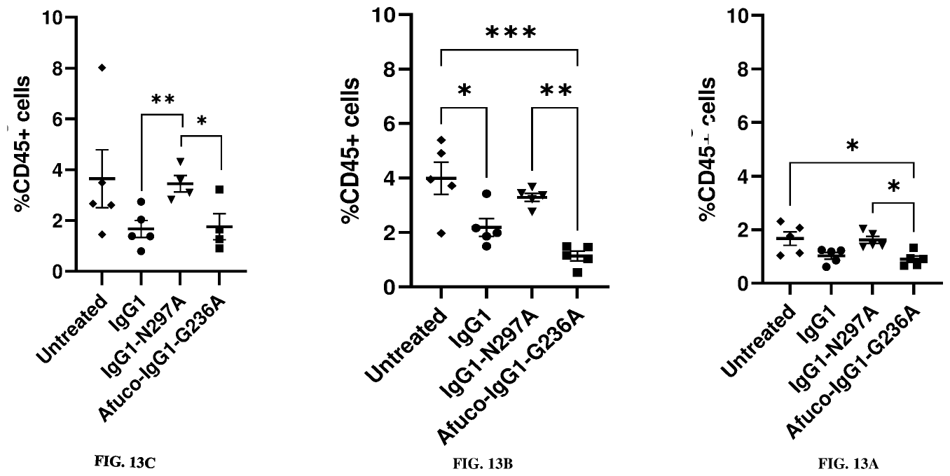

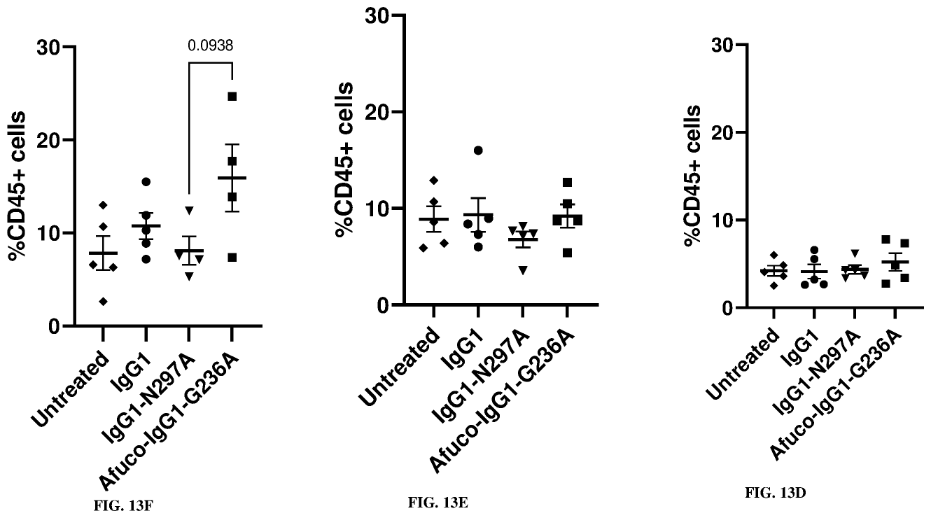

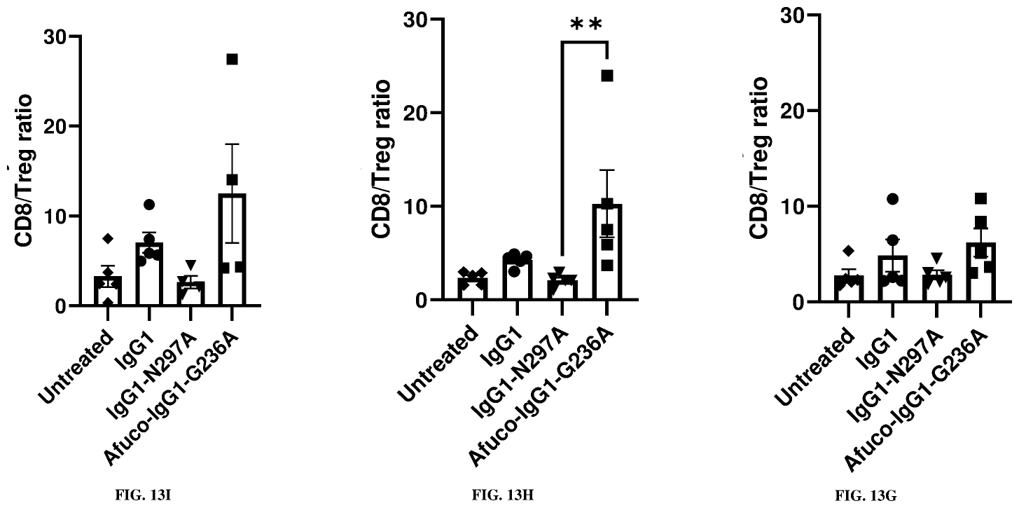

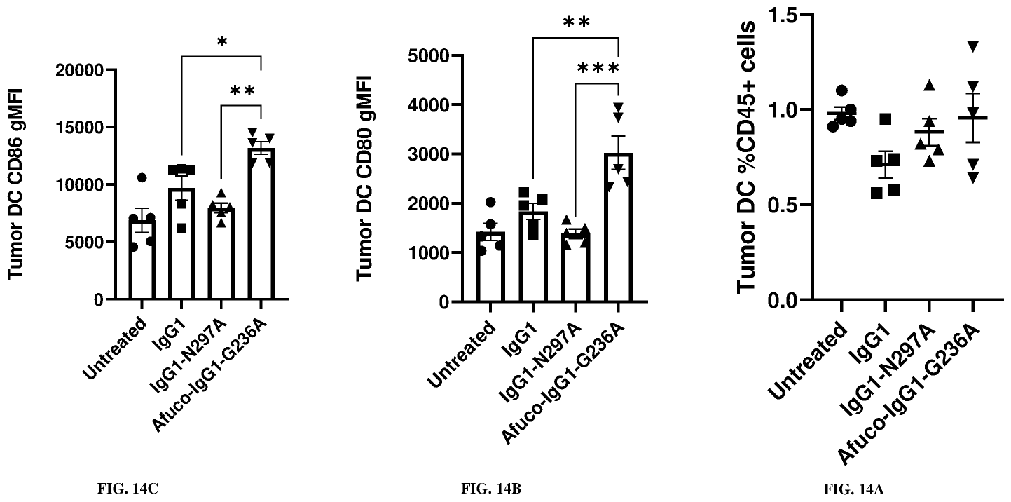

Following the characterization of the Fc-engineered antibody variants, Afuco-IgG1-G236A was found to be a remarkably efficient and suitable human IgG scaffold of GITR mAb, leading to enhanced antitumor activity, the effect of the anti-GITR Fc region variants on the TME was assessed. Humanized FcγR mice with refractory MC38 tumors were treated with 100 μg/mice/injection intraperitoneal (IP) of anti-mGITR hIgG1 Fc region variants, Afuco-IgG1-G236A (GA-aFuc), IgG1 Fc-silent N297A (NA), IgG1, untreated mice were used as control. Mice were sacrificed and tumors were harvested at three time points following treatment, 1 day, 4 days (total of 2 mAb administrations) and 8 days (total of 3 mAb administrations). For the assessment of the TME composition, single cell suspensions were generated and analyzed for the percentages of lymphocytes by flow cytometry. The flow cytometry results for the frequency of Tregs in TMEs of mice 1-, 4- and 8-days post onset of anti-GITR Fc region variants treatment are presented in FIGS. 13A-13C, respectively, Tregs were detected for CD45+CD3+CD11b−CD8−CD4+FoxP3+. The flow cytometry results for the frequency of CD8+ cells in TMEs of mice 1-, 4- and 8-days post onset of anti-GITR Fc region variants treatment are presented in FIGS. 13D-13F, respectively, CD8+ cells were detected for CD45+CD3+CD11b−CD8+CD4−. CD8/Tregs ratios in TMEs of mice 1-, 4- and 8-days post onset of anti-GITR Fc region variants treatment are presented in FIGS. 13G-13I, respectively. FIGS. 13A-13I, untreated mice (untreated, rhombus), IgG1 (circle), IgG1-N297A upside down triangle), and Afuco-IgG1-G236A (square). An unpaired two-tailed t-test was used. Data are represented as mean±SEM.

As can be seen from FIGS. 13A-13I, a decrease in Treg frequency after treatments with all Fc region variants except the IgG1 Fc-silent N297A (NA) was apparent, which indicates a Fc-mediated depletion of intra-tumoral Tregs. The Treg frequency decrease occurs at early time point (1 day) with the Fc-enhanced variant Afuco-IgG1-G236A (GA-aFuc), while CD8+ T cell frequency remains the same at this early time point. Notwithstanding, at the later time point (8 days) an increase in CD8+ T cell frequencies was observed with the Fc-enhanced variants, but not by IgG1. GA-aFuc demonstrated the highest CD8/Treg ratio at an early time point (4 days), while other variants demonstrated at a later time point. CD8/Treg ratio showed a significant increase at early time point only for the GA-aFuc treatment compared to IgG1-N297A, while at a later time point the increase is also observed between IgG1 Fc region variants compared to IgG1-N297A.

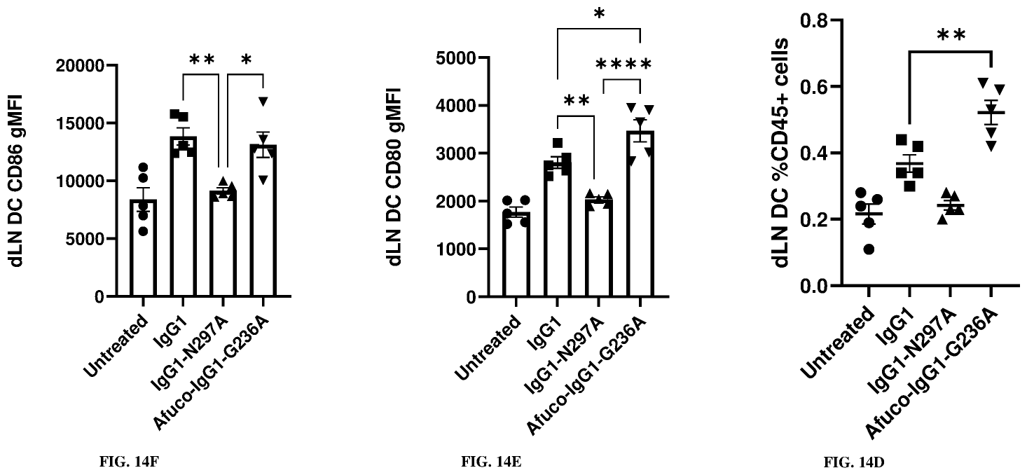

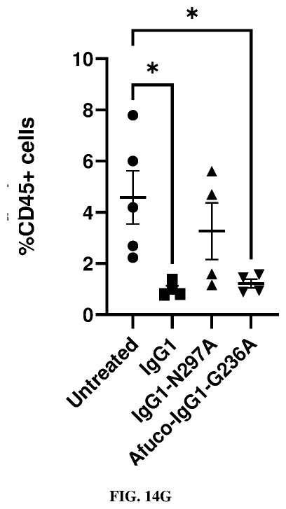

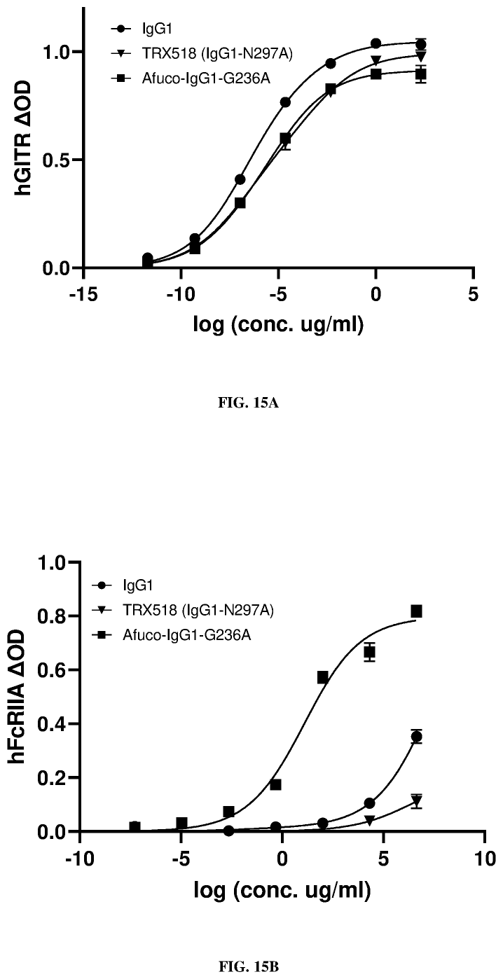

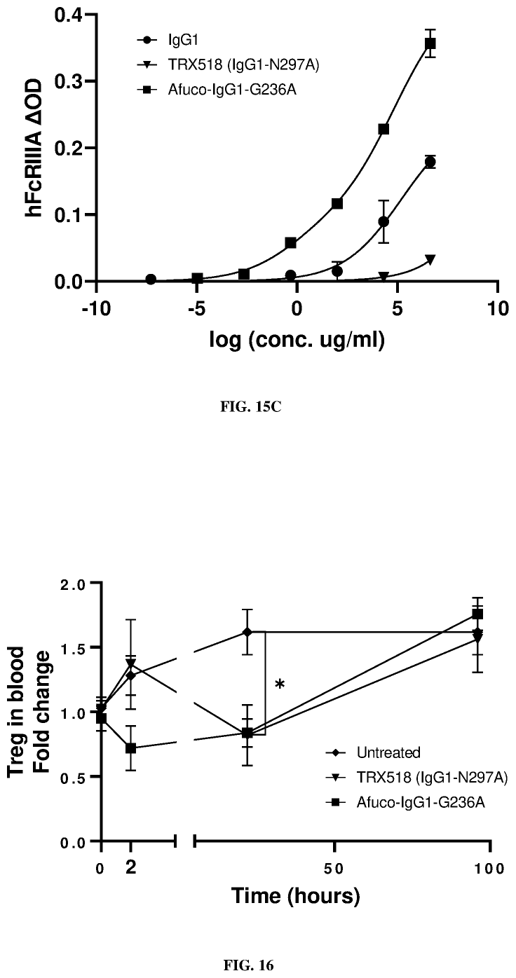

DC activation state within the TMEs and dLNs was assessed by measuring DCs and DC's activation markers in TMEs and dLNs of humanized FcγR inoculated with MC38 cells and treated with human anti GITR Fc region variants Afuco-IgG1-G236A (GA-aFuc), IgG1 Fc-silent N297A (NA), and IgG1. Tumors and dLNs from untreated mice served as control. Tumors and dLNs were harvested 4 and 8 days after mAb treatment onset, and then single cell suspensions were generated from the harvested organs and analyzed by flow cytometry. FIGS. 14A-14F flow cytometry for the detection of DCs and activated DCs in TMEs (14A-14C) and dLNs (14D-14F), 4 days after the onset of treatment with either Afuco-IgG1-G236A (GA-aFuc, upside down triangle), IgG1 Fc-silent N297A (IgG1-N297A, triangle), and IgG1 (square). Untreated mice served as control (untreated, circle). FIG. 14A presents, the percentages of DCs in the TMEs detected for CD45+ CD11b− NK1.1− F4/80− CD11c+ MHCII+. FIG. 14B presents, the geometric mean fluorescence intensity (gMFI) of activated CD80 DCs in the TMEs detected for CD45+ CD11b− NK1.1− F4/80− CD11c+ MHCII+. FIG. 14C presents, gMFI of activated CD86 DCs in the TMEs detected for CD45+ CD11b− NK1.1− F4/80− CD11c+ MHCII+. FIG. 14D presents, the percentages of DCs in dLNs, detected as described for FIG. 14A. FIG. 14E presents, the geometric mean fluorescence intensity (gMFI) of activated CD80 in dLNs DCs detected as described for FIG. 14B. FIG. 14F presents, gMFI of activated CD86 DCs in dLNs, detected as described for FIG. 14C. FIG. 14G presents, the percentages of DCs in TMEs of treated mice, as described for FIG. 14, 8 days after the onset of treatment. Geometric mean fluorescence intensity (gMFI) was calculated using FLOWJO software. Ordinary one-way ANOVA using Tukey's multiple comparisons statistical test was used. Data are represented as mean±SEM.

As can be seen from FIGS. 14A-14G, DCs percentages in the TME decreased between day 4 and day 8 post treatment onset (FIGS. 14A and 14G). Increased DCs percentages were observed in the dLN, 4 days post treatment onset compared to that observed with the IgG1 variant (FIG. 14D). Although all Fc region variants beside the IgG1 Fc-silent N297A (NA) exhibited increased density of CD80 or CD86 activation markers within the TMEs and dLNs (14B-14C and 14E-14F), the Afuco-IgG1-G236A (GA-aFuc) variant demonstrated the highest increase for both markers.

### Example 7. Impact of Anti-Human GITR hIgGs on the Tumor Microenvironment (TME) and Draining Lymph Node (dLN) Immune Landscape

To test whether the optimized Fc region variant (Afuco-IgG1-G236A) keeps its enhanced anti-tumor activity while targeting hGITR, the heavy and light chains of anti hGITR humanized antibody TRX518) were synthesized and cloned into mammalian expression vector to form the anti hGITR humanized antibody. The G236A and N297A mutations were introduced by site-directed mutagenesis into the CH2 domain to generate TRX518 (N297A) and Afuco-IgG1-G236A Fc region variant of the same GITR mAb clone with enhance binding to the activating hFcγRs. The amino acid sequence of the heavy chain constant region, of the Afuco-IgG1-G236A variant, in which Alanine in position 236 substituted Glycine, is set forth in SEQ ID NO: 13. The light chain constant region of this variant, as well of the TRX518 is according to SEQ ID NO: 14.

Following production, the binding affinities of anti hGITR (anti hGITR) Fc region variants were measured and verified by comparative ELISA in which OD450 values were plotted against increasing concentrations of tested antibodies to assess binding to plate-bound protein. FIG. 15A present the ELISA results for the binding of the humanized tested antibodies, IgG1 (circle), hGITR TRX518 (TRX518 (IgG1-N297A), upside down triangle), Afuco-IgG1-G236A (squares) to hGITR. The binding affinities of the Fc region variants to the hFcγRs members, RIIA and RIIIA were also measured by ELISA. 15B-15C, present the ELISA results for the binding of the tested antibodies, IgG1 (circle), hGITR TRX518 (TRX518 (IgG1-N297A), upside down triangle), Afuco-IgG1-G236A (squares) to FcγRIIA and FcγRIIIA, respectively.

The results depicted in FIG. 15A indicate that the different Fc scaffolds of anti hGITR TRX518 preserved the binding affinity to hGITR with comparable binding affinity. The lack of binding to the two human FcγRs by TRX518 (IgG1-N297A, (triangle) is apparent, while “Afuco-IgG1-G236A” (square) demonstrated increased binding to both human FcγRIIA and FcγRIIIA.

### Example 8. FcγR Dependent Tumor Infiltrating Lymphocytes (TILs) Modulation Following Anti GITR Treatment

To assess the influence of humanized anti hGITR Fc region variants on modulation of immune cells in the blood and within the TME, mice humanized for FcγR and GITR (hFcγR/hGITR) were generated.

Humanized FcγR/hGITR mice with refractory MC38 tumors were treated with the anti hGITR antibody TRX518 (IgG1-N297A), and with its Fc region variant Afuco-IgG1-G236A. Treg levels in blood were assessed over time following one administration of the anti hGITR Fc region variant. FIG. 16 presents the results for blood Tregs frequencies post treatment with anti-hGITR antibody TRX518 (IgG1-N297A, upside down triangle) Afuco-IgG1-G236A (square), untreated mice as control (untreated, rhombus). Blood Treg frequency decrease occurred similarly, 22 hours post treatment with both Fc-silent IgG1-N297A (triangle) and Afuco-IgG1-G236A (square) variants but Treg frequency decrease occurred at an earlier time point (2 hours) following treatment with Afuco-IgG1-G236A than following treatment with IgG1 Fc-silent N297A. Four days from treatment onset Treg level recovered in blood.

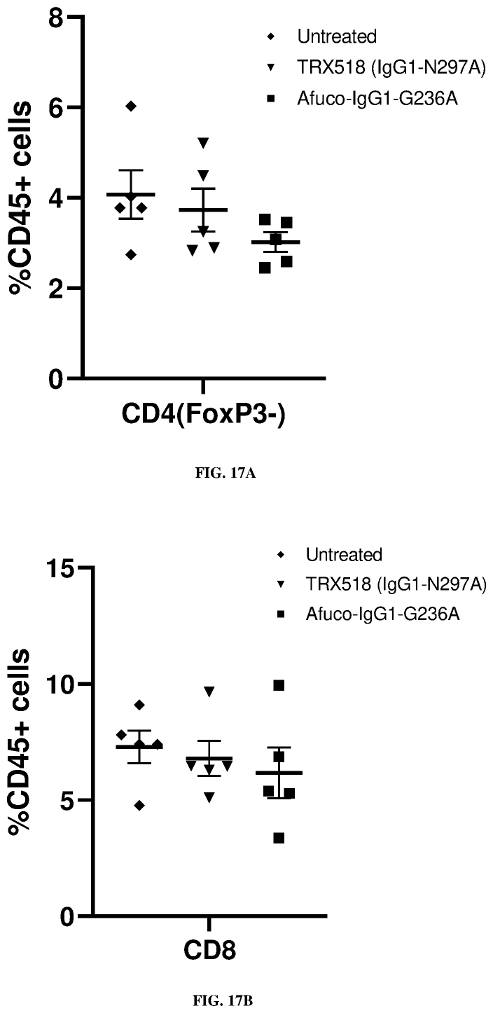

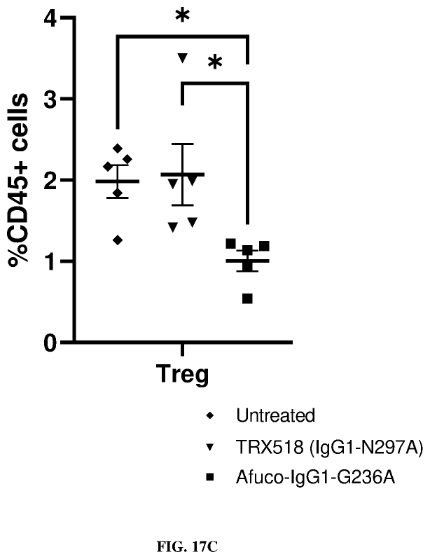

Next, the TME composition was assessed, tumors were harvested after 4 days from treatment onset (after 2 antibody administrations), and single cell suspensions were generated and analyzed by flow cytometry for modulation of the immune compartment in the TME. FIGS. 17A-17C present flow cytometry of the effect of the different Ab variants on the percents of CD4+ FoxP3, CD8+, and Tregs in the TME, respectively. TRX518-IgG1-N297A (TRX518 (IgG1-N297A, upside down triangle), or Afuco-IgG1-G236A (squares), untreated mice as control (untreated, rhombus). Unlike the Treg depletion in the blood and in the huFcγR/GITR in-vivo model described above for TRX518-IgG1-N297A, Treg depletion within the TME was exclusive to the Afuco-IgG1-G236A treated group (FIG. 17C), while no changes were observed in the other TILs (FIGS. 17A-B). Anti hGITR mediated-Treg depletion was found to be FcγR dependent, in contrast to the Fc silent antibody TRX518, currently in clinical trials.

### Example 9. Treg Depletion by Anti GITR Afuco-IgG1-G236A Variant is NK-Independent

For the in vivo NK depletion assay, humanized FcγR mice were challenged with refractory MC38 tumors were treated twice (at day 0 and day 3) with 100 μg/mice/injection IP, anti-mGITR hIgG1 Fc region variants, Afuco-IgG1-G236A (GA-aFuc) and IgG1 Fc-silent N297A (NA), while depleting NK cells. The humanized FcγR mice were challenged with refractory MC38 tumor cells 14 days (day −14) before the first treatment with the anti-mGITR hIgG1 Fc region variants (day 0). Depletion of the NK cells with αNK1.1 (250 ug, #BE0036, BioXCell) was performed 1- and 2-days before and after the first treatment with the anti-mGITR hIgG1 Fc region variants (days −2, −1, 1, and 2). NK cells absence was verified on day 0 and day 4. Mice treated with PBS or PBS and αNK1.1 were used as control. On days 0 and 4 following the onset of the anti-mGITR hIgG1 Fc region variants treatment, blood was drawn from the tested animals and analyzed for NK cell presence by flow cytometry. Additionally, single cell suspensions generated from tumors harvested on day 4 following the onset of the treatment, were analyzed for the modulation in the immune compartment in the TME by flow cytometry.

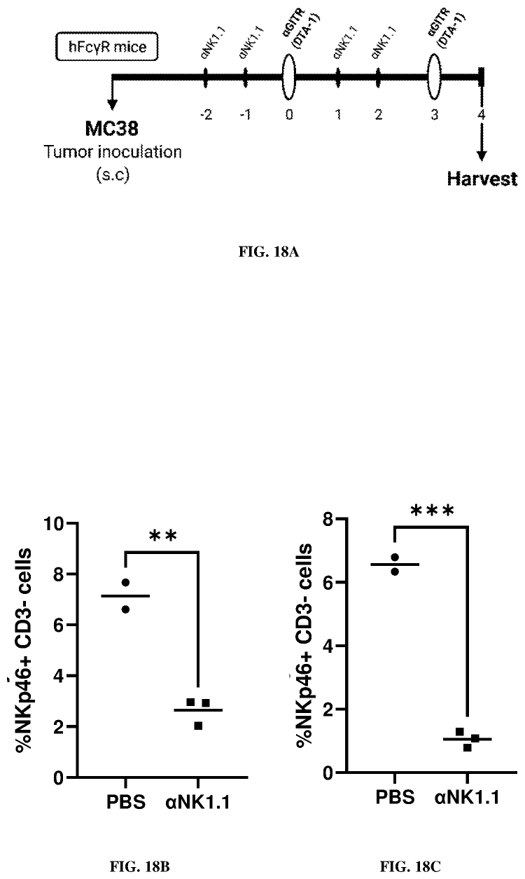

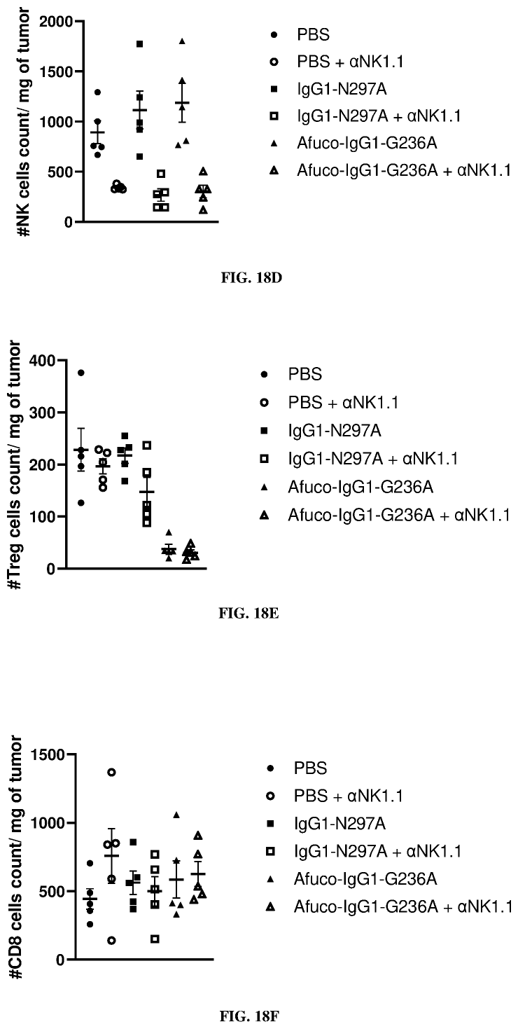

FIG. 18A presents the timeline from the inoculation of refractory MC38 tumor cells into mice to day 4 from the onset of anti-mGITR hIgG1 Fc region variants treatment when blood was drawn and tumors were harvested. FIGS. 18B-18C, present flow cytometry of NK cell in the blood samples from mice on days 0 and 4 from onset of anti-mGITR hIgG1 Fc region variants treatment and following NK cells depletion, respectively. NK cells were detected for CD45+CD3-NKp46+, blood from mice treated with PBS only served as negative control. FIGS. 18D-18F, present, cells count per mg tumor of NK cells (detected for CD45+CD3−NKp46+), Treg cells (detected for CD45+CD3+CD11b−CD8−CD4+FoxP3+), and CD8+ cells (detected for CD45+CD3+CD11b−CD8+CD4−), respectively. Mice treated with PBS (PBS, full circle), Mice treated with PBS and αNK1.1 (PBS+αNK1.1, empty circle), IgG1-N297A (full square), IgG1-N297A and αNK1.1 (IgG1-N297A+αNK1.1, empty square), Afuco-IgG1-G236A (full triangle), Afuco-IgG1-G236A and αNK1.1 (Afuco-IgG1-G236A+αNK1.1 empty triangle). An unpaired two-tailed t-test was used. Data are represented as mean±SEM.

As can be seen from FIGS. 18B-18C, a significant depletion of NK cells was achieved in αNK1.1 injected mice as early as day 0 of the onset of anti-mGITR hIgG1 Fc region variants treatment, compared to negative control mice injected with PBS only. Between day 0 and day 4 of the onset of anti-mGITR hIgG1 Fc region variants treatment, NK cells' depletion was even significantly broader in αNK1.1 injected mice. As can be seen from FIGS. 18D-18F, the absolute number of intra-tumoral NK cells show depletion in all αNK1.1 injected groups, compared to the PBS injected control mice. In the afuco-IgG1-G236A (GA-aFuc) treated group, Treg cells were depleted in an independent manner from NK cells, in the IgG1 Fc-silent N297A (NA) treated group much moderated depletion of Treg was observed. CD8+ cells levels were conserved 4 days after treatment onset in mice groups treated with either afuco-IgG1-G236A (GA-aFuc) or the IgG1 Fc-silent N297A (NA). The in vivo NK depletion assay demonstrated that Treg depletion is independent of NK presence.

### Example 10. Generation and Characterization of Non-Agonistic Bispecific Afuco-IgG1-G236A Anti GITR Antibodies

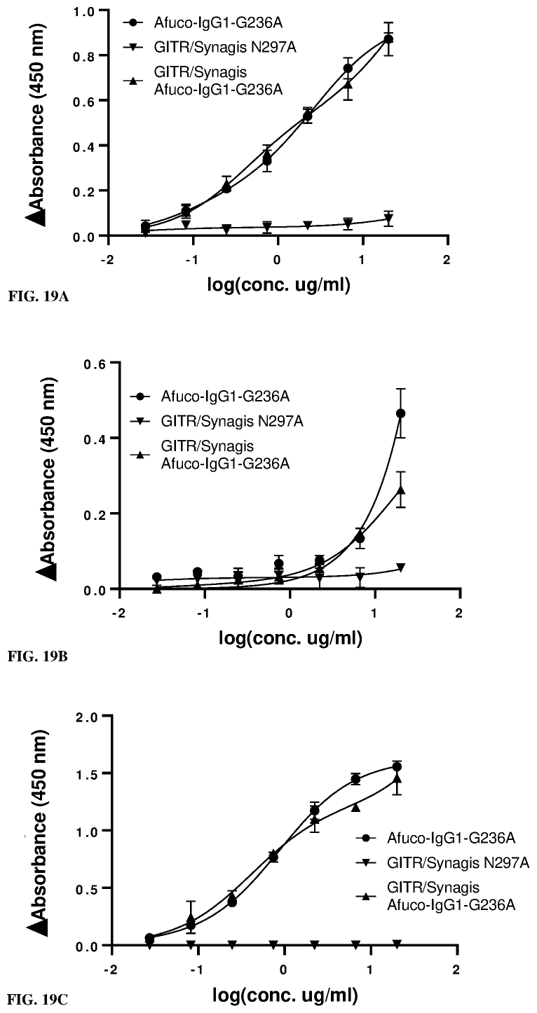

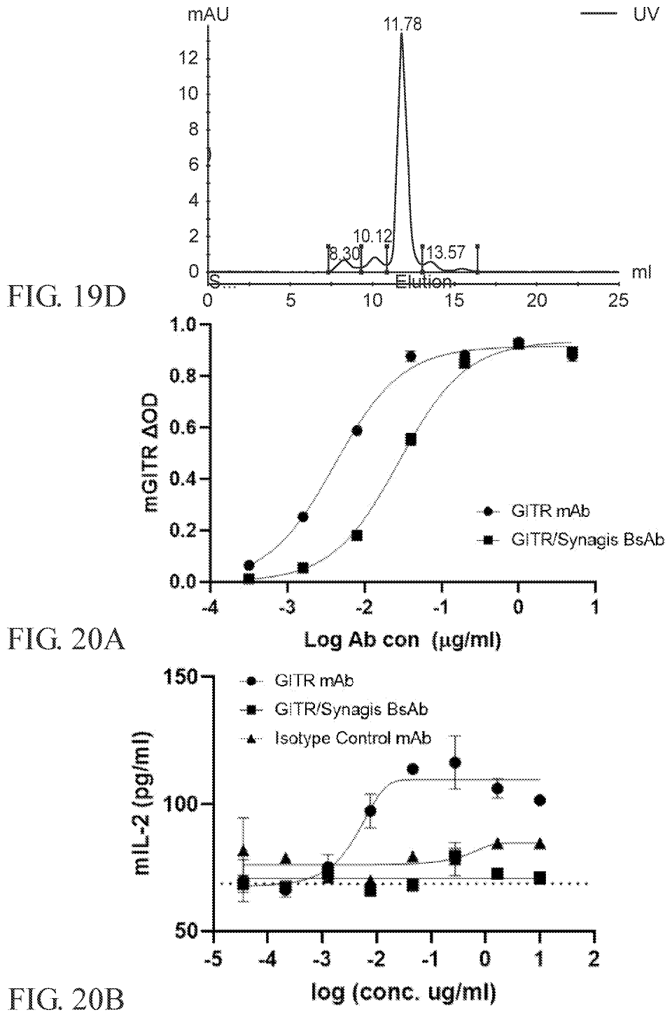

For the assessment of the necessity of bivalent GITR agonism in tandem with enhanced FcγR targeting, non-agonistic bispecific antibodies were generated, using conventional methods, based on the Fc scaffolds of Afuco-IgG1-G236A and IgG1-N297A and on the Fab of Synagis (Palivizumab), a humanized anti-RSV (respiratory syncytial virus). The Afuco-IgG1-G236A Fc scaffold and Synagis Fab were used to generate GITR/Synagsis Afuco-IgG1-G236A bispecific antibody and IgG1-N297A and Synagis Fab were used to generate GITR/NA Synagis bispecific antibody. The novel bispecific antibodies were purified and characterized by ELISA, High-performance liquid chromatography (HPLC), and Mass-spectrometry. For the HPLC, samples of the two bispecific non-agonistic Abs were digested with trypsin using the S-trap method. The resulting peptides were analyzed using nanoflow liquid chromatography (nanoAcquity) coupled to high resolution, high mass accuracy mass spectrometry (Exploris). Each sample was analyzed on the instrument separately in a random order in discovery mode. The data was processed using Byonic search engine against the IgG1 peptide and the Fc glycan library. Data as quantified using Skyline and validated manually. The binding of the bispecific antibodies to FcγRs, FcγRIIA, FcγRIIB and FcγRIIIA was measured. OD450 values obtained in the ELISA test were plotted against increasing concentrations of indicated antibody to assess binding to plate-bound protein. The monospecific afuco-IgG1-G236A was used as control. FIGS. 19A-19C present ELISA results for the binding of the bispecific non-agonist variants, Afuco-IgG1-G236A (circle), GITR/Afuco-IgG1-G236A Syn (DTA1/Syn GA-aFuc, triangles) and GITR/NA Syn (DTA1/Syn, upside down triangles) to the different hFcγRs, FcγRIIA, FcγRIIB and FcγRIIIA, respectively. The monospecific Afuco-IgG1-G236A (DTA1 GA-aFuc) was used as a control (circles). FIG. 19D presents, HPLC analysis of the trypsin digested GITR/Afuco-IgG1-G236A Synagis non-agonistic antibody. Data are represented as mean±SEM.

As can be seen from FIGS. 19A-19D, The GITR/Afuco-IgG1-G236A Synagis humanized bivalent (bispecific) and monovalent Afuco-IgG1-G236A (monospecific) antibodies possess similar binding abilities to the different hFcRs. Nevertheless, the GITR/Afuco-IgG1-G236A Synagis showed preferential binding of the Fc domain to activating Fc receptors. The HPLC analysis revealed a protein purity of 80%. In addition, Mass-spectrometry analysis revealed a yield of ˜73.13% non-fucosylated forms of GITR/Afuco-IgG1-G236A Synagis. A bispecific anti mouse GITR/Synagis was generated with high purity and with the desire binding property by both Fab and Fc domains.

### Example 11. In Vitro Activity Assay of Anti-Mouse-GITR/Synagis Bispecific mAbs

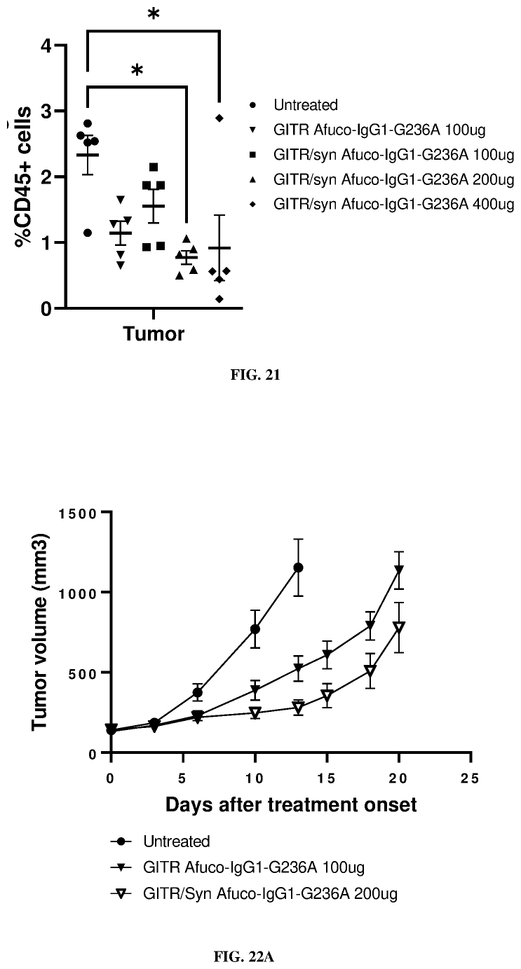

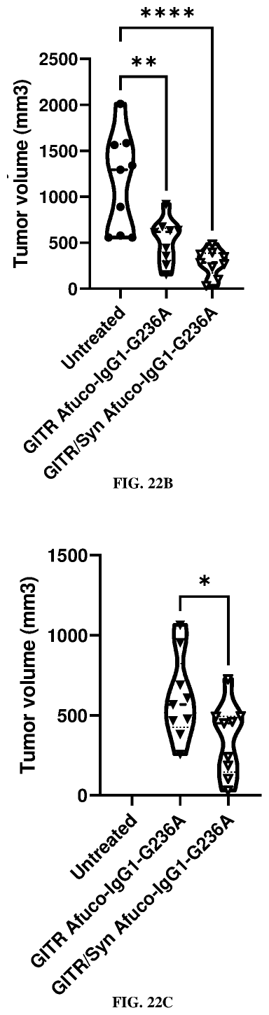

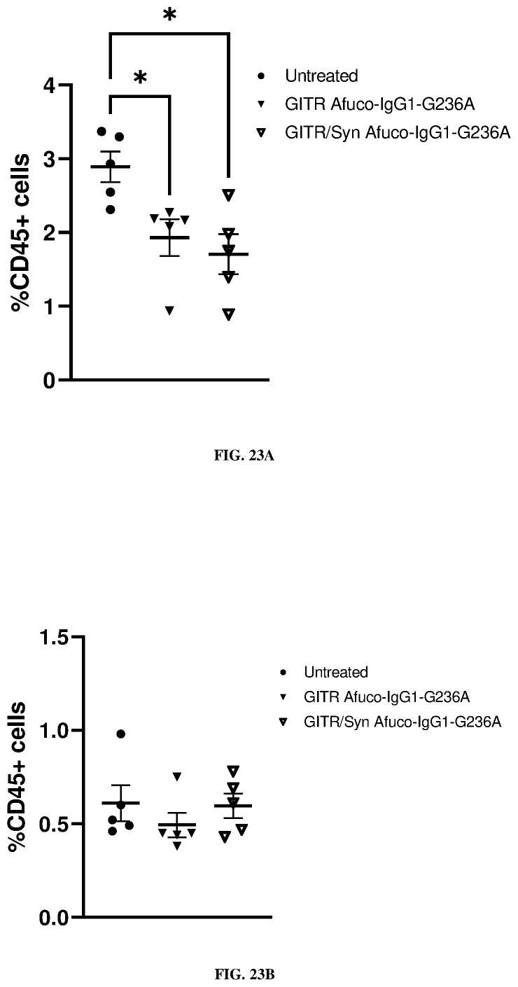

Anti-mouse GITR (mGITR) non agonist bispecific mAb with the N297A substitution in the Fc, denoted mGITR/NA Synagis was generated. The novel mGITR/NA Synagis capacity to bind mGITR was assessed by ELISA compared to the monospecific mIgG1-N297A. Optical Density 450 (OD450) values were plotted against increasing concentrations of mGITR/NA Synagis and mIgG1-N297A to assess binding to plate-bound protein. Next, isolated T cells from WT) mice were incubated with anti-CD3 and increasing concentrations of anti mGITR/NA Synagis, or the monospecific mIgG1-N297A. A non-GITR binding antibody with the N297A substitution in the Fc region served as isotype control. ELISA was performed on the supernatant, to detect mouse IL-2 secreted by the T cells, 24 hours post incubation. The IL-2 ELISA was Perform using Biolegened ELISA MAX™ Deluxe Set Mouse IL-2 (BLG-431004) according to manufacturer's instructions. Data are represented as mean±SEM. FIG. 20A present the novel mGITR/NA Synagis (squares) binding capacity of mGITR compared to the monospecific mIgG1-N297A (full circles). FIG. 20B presents murine IL-2 (mIL-2) secretion (pg/ml) by T cells (activated with anti-CD3 antibodies) following their incubation with increasing concentrations of anti mGITR/NA Synagis (squares), the monospecific mIgG1-N297A (full circles), or a non-GITR binding antibody with the N297A substitution in the Fc region (Isotype control NA) as an isotype control (isotype control NA, triangles). The dotted line represents the natural threshold for IL-2 concentration following the activation with anti-CD3 and without any Ab treatment.

As can be seen in FIGS. 20A-20B, the bispecific mGITR/NA Synagis possess lower binding capacity to mGITR compared to the monospecific mIgG1-N297A. Already at antibodies' concentration of ˜10³ μg/ml, the OD450 of mGITR/NA Synagis is significantly lower (0.18) compared with mIgG1-N297A (0.6). A saturation point is reached by mIgG1-N297A at ˜10-1.5 μg/ml with a plateau with OD450 value of ˜0.9, while mGITR/NA Synagis reached the same OD value and a plateau at antibodies' concentration of ˜100 μg/ml. This difference coincides with the antibodies' valences, each bispecific mGITR/NA Synagis antibody capable of binding only one mGITR molecule, while each monospecific mIgG1-N297A antibody capable of binding two mGITR molecules.

mGITR/NA Synagis did not elicit mIL-2 secretion by anti-CD3 activated murine T cells at increasing concentration. The mIL-2 levels secreted following the incubation of T cells with the mGITR/NA Synagis were even lower the non-GITR binding, isotype control antibody. The lack of ability of mGITR/NA Synagis to elicit IL-2 secretion by T cells indicates the lack of agonistic activity. However, incubation with mIgG1-N297A elicited mIL-2 secretion by anti-CD3 activated murine T cells in a dose dependent manner at antibodies concentration between ˜10⁻³ to ˜10^{-1.5} μg/ml, when the secretion levels stayed the same despite increasing concentrations of mIgG1-N297A. At the concentration range of ˜10⁻³ to ˜10^{1.5} μg/ml, mIgG1-N297A showed an agonist activity by binding mGITR and IL-2 secretion by activated T cells in a dose dependent manner. The bispecific technique, enabled the generation of a non-agonist anti GITR.

### Example 12. Anti-GITR Variants, GA-aFuc and Bispecific Non-Agonistic Humanized afuco-IgG1 Exhibit In Vivo Anti-Tumor Activity

Humanized FcγR mice were challenged with refractory MC38 tumors. 14 days post challenge, mice were treated with 100 μg mouse anti-DTA-1 (anti-GITR) GA-aFuc Ab or with 100 μg, 200 μg, or 400 μg of a humanized GA-aFuc Fc region variant of the non-agonistic bispecific antibody (GITR/syn GA-aFuc) Ab. Untreated mice served as negative control. Tumors were harvested 24 hr post treatment, single cell suspensions were generated and analyzed for Tregs modulation in the TME by flow cytometry. FIG. 21 presents, flow cytometry analysis of the percent of Tregs in the TME following the treatment with 100 μg (square), 200 μg (triangle), or 400 μg (rhombus) of a GITR/syn GA-aFuc, and with 100 μg GA-aFuc (upside down triangle). Untreated mice served as control (untreated, circle). Tumors from untreated nice were analyzed as well as a negative control. Tregs were detected by CD45+CD3+CD11b−CD8−CD4+FoxP3+. Data are represented as mean±SEM. (n=5 mice/group, one-way ANOVA).

As can be seen from FIG. 21, both non-agonistic and agonistic aGITR antibodies showed a significant ability to immunomodulate the TME by in vivo Tregs depletion. The reduction in Treg percentages in the TME correlated to the concentration of the novel non-agonistic GITR/syn GA-aFuc.

### Example 13. Agonistic and Non-Agonistic Humanized Afuco-IgG1 GA Variants Exhibit in Vivo Anti-Tumor Activity

To further assess the in vivo anti-cancer immunity of GITR/syn GA-aFuc and GA-aFuc, hFcγR mice with refractory MC38 tumors were treated 14 days post tumor cells inoculation with GA-aFuc Ab or with the non-agonistic GITR/syn GA-aFuc Ab and monitored for tumor progression (tumor volume) for 20 days. Untreated mice served as a control, and were sacrificed on day 13 to prevent further suffering by the animals FIGS. 22A-22C present the results of the tumor volume (mm³) progression in hFcγR mice with MC38 tumors following treatment with 100 μg GA-aFuc Ab (full triangles) or with 200 μg of the non-agonistic GITR/syn GA-aFuc Ab (empty triangles). Untreated mice served as a control (full circles), n=9 per each tested group. FIG. 22A presents, 20 days tumor volume measurements. FIG. 22B presents, tumors volumes of mice of all groups on day 13 from treatment onset. FIG. 22C presents, tumors volumes of mice of GA-aFuc Ab and GITR/syn GA-aFuc Ab tested groups on day 15 from treatment onset. Untreated mice were sacrificed on day 13 and do not appear in this figure.

From FIGS. 22A-22C it is clear that both non-agonistic aGITR Abs, GITR/syn GA-aFuc and GA-aFuc demonstrated successful anti-tumor efficacy in-vivo. However, the non-agonist GITR/syn GA-aFuc exhibited a significantly higher cancer immunity compared to the agonistic aGITR antibody GA-aFuc. And so, the non-agonist aGITR variant showed an increased ability to suppress tumor growth in vivo, compared to a matched Fc agonistic aGITR mAb.

### Example 14. In Vivo Dendritic Cells Activation by Agonistic and Non-Agonistic Humanized afuco-IgG1 GA Variants

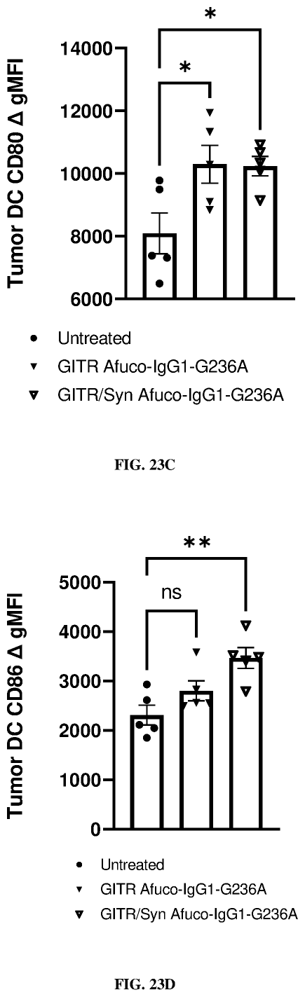

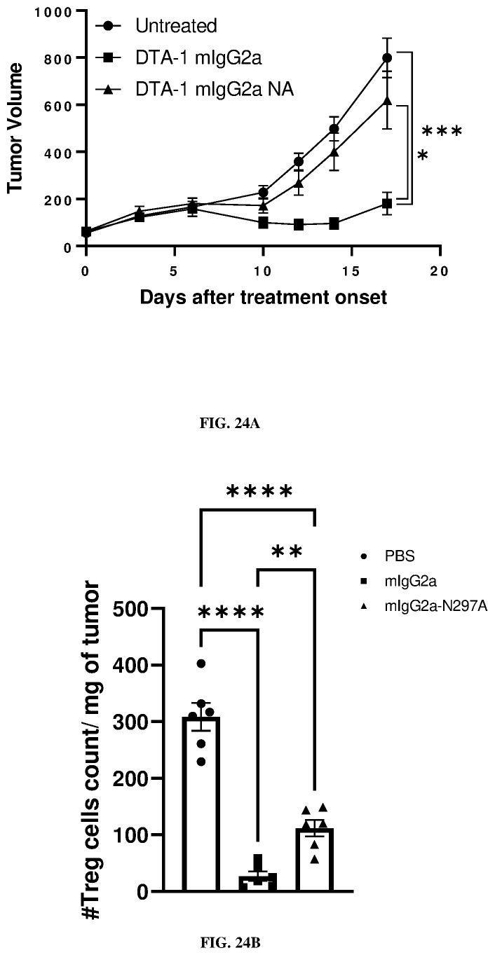

Humanized FcγR mice were inoculated with refractory MC38 tumors. The mice were treated twice, 0- and 3-days post treatment onset with 100 μg GA-aFuc Ab, or with 200 μg, GITR/syn GA-aFuc Ab. Untreated mice served as negative control. Tumors were harvested 24 hr following the second treatment, single cell suspensions were generated and analyzed for of the TME, namely, Tregs depletion and dendritic cells (DCs) activation, by flow cytometry. FIGS. 23A-23B present, flow cytometry analyses of the percent of Tregs and DCs in the TME, respectively, following the treatment with 200 μg of a GITR/syn GA-aFuc (GITR/syn Afuco-IgG1-G236A upside down empty triangles), and with 100 μg GA-aFuc (Afuco-IgG1-G236A upside down full triangles). Tumors from untreated nice were analyzed as well as a negative control (circles). Tregs were detected by CD45+CD3+CD11b−CD8−CD4+FoxP3+ and DCs were detected by CD45+ CD11b− NK1.1− F4/80− CD11c+ MHCII+. Data are represented as mean±SEM. (n=5 mice/group, one-way ANOVA). FIGS. 23C-23D present flow cytometry analyses of the activation of DCs by detecting the percent of CD80+ or CD86+] expressing DCs, respectively. GITR/syn Afuco-IgG1-G236A (upside down empty triangles), Afuco-IgG1-G236A upside down full triangles, tumors from untreated nice were analyzed as well as a negative control (circle). Geometric mean of the fluorescent intensities (gMFI) was calculated using FLOWJO software. DCs were detected for by CD45+ CD11b− NK1.1− F4/80− CD11c+ MHCII+, CD80+ DCs were detected with the addition of anti-CD80, CD86+ DCs were detected with the addition of anti-CD86. Data are represented as mean±SEM, each dot represents an individual mouse (n=5 mice/group, one-way ANOVA).

FIGS. 23A-23B show that while Tregs underwent significant depletion following the treatment with either aGITR Abs, GITR/syn GA-aFuc and GA-aFuc compared to control mice, DCs levels were stable. Indicating that the agonistic and/or non-agonistic activity of the aGITR Abs does not affect or modulate the levels of DCs.

FIGS. 23C-23D show that treatment with either GITR/syn GA-aFuc or GA-aFuc Abs increased CD80+ DCs significantly. Treatment with GITR/syn GA-aFuc Abs increased remarkably increased the levels of CD86+ DCs, a lesser increase of CD86+ DCs followed the treatment with GA-aFuc Abs. Fc region mediated DCs activation was exhibited by both GITR/syn GA-aFuc or GA-aFuc Abs.

### Example 15. In Vivo Anti-Cancer Activity of Mouse aGITR-mIgG2a Variants in Wild Type Mice

Anti-GITR-mIgG2a Abs that has preferential binding to activating murine receptors FcγRI and FcγRIV were assessed for their anti-cancer activity. Wild type (WT) C57BL/6J mice with MC38 tumors were treated 8 days post tumor cells inoculation with 100 μg mouse anti-GITR (DTA-1) Ab (DTA-1 mIgG2a) or 100 μg mIgG2a-N297A Ab (DTA-1 mIgG2a NA) with the silent mutation N297A in the Fc region of mIgG2a is the parallel mouse Fc with enhancement to the activating Fc receptors. Tumors' progression was monitored by measuring tumors volumes for 17 days. Untreated mice were used as control.

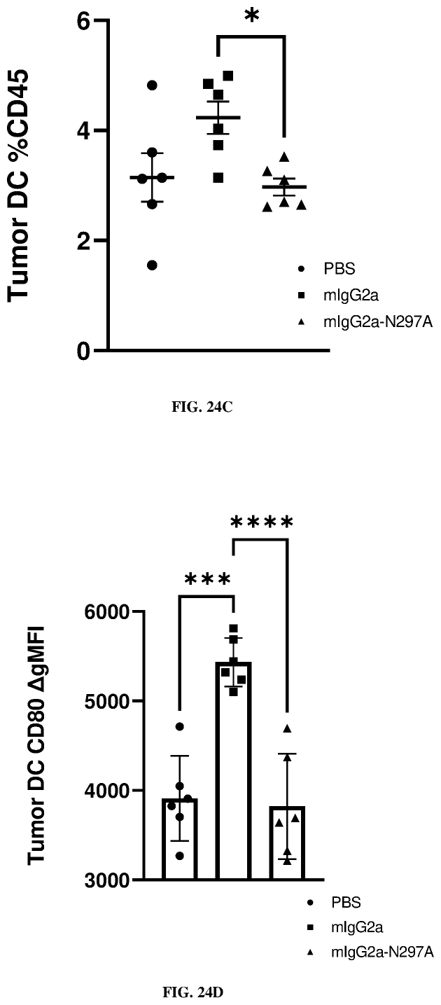

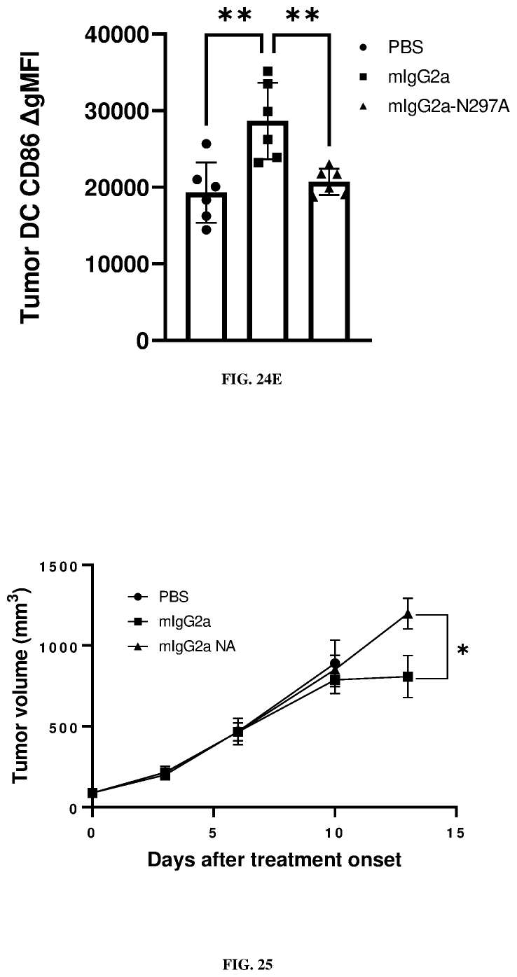

FIG. 24A present the results of 17 days tumor volume progression surveillance in WT mice with MC38 tumors following treatment with 100 μg DTA-1 mIgG2a (squares) or with 100 μg of DTA-1 mIgG2a NA Ab (triangles). Untreated mice served as a control (full circles). Data are represented as mean±SEM, n=9 mice/group

As can be seen from FIG. 24A, Mice treated with 100 μg of the Fc-engaging DTA-1 mIgG2a Ab, showed statistically significant decreases in tumor growth and complete response when comparing to mIgG2a-N297A

Next, WT mice with MC38 tumors were treated with 100 μg of DTA-1 mIgG2a Ab and 100 μg of DTA-1 mIgG2a NA, while untreated mice served as negative control. Tumors were harvested 4 days post treatment and single cell suspensions were generated and analyzed for modulation in the immune compartment in the TME by flow cytometry. the immunomodulation parameters that were assayed were Tregs count/mg tumor (detected using anti-FoxP3) and DCs percentages out of total lymphocyte (detected using anti-CD11c/anti-CD45), DCs activation status (detected using anti-CD80 or anti-CD86). FIG. 24B-24E present, flow cytometry analyses of the above detailed parameters, in harvested tumors of WT mice treated with 100 μg of DTA-1 mIgG2a Ab (mIgG2a, squares) and 100 μg of DTA-1 mIgG2a NA (mIgG2a-N297A, triangles) or of untreated mice (PBS, circles), used as a control. Each dot represents an individual mouse n=6 mice/group, and data are represented as mean±SEM and analyzed using one-way ANOVA. Delta gMFI was calculated using FLOWJO software. Unpaired t-test was used. FIG. 24B presents, Tregs count/mg tumor detected using anti-FoxP3 in all tested groups. FIG. 24C presents, DCs percentages out of total lymphocyte (detected using anti-CD11c/anti-CD45) in all tested groups. FIG. 24D presents, delta gMFI of CD80+ activated DCs in all tested groups. FIG. 24E presents, delta gMFI of CD86+ activated DCs in all tested groups.

As can be seen from FIG. 24B-24E, both DTA-1 mIgG2a and DTA-1 mIgG2a NA elicited a remarkable and significant Tregs depletion in vivo, compare to untreated mice. Furthermore, the DTA-1 mIgG2a Ab elicited significantly greater Tregs depletion compared to the Fc-null antibody, mIgG2a NA, indicating that the specificity and the Fc interaction of the Ab contribute to the immunomodulation abilities of the Ab. The in vivo treatment with DTA-1 mIgG2a Ab modulated the TME immune cell population, increasing DCs percentages significantly compared to the DTA-1 mIgG2a NA, suggesting that the Fc interaction is crucial for the increase of DCs in the TME. Additionally, in vivo treatment with DTA-1 mIgG2a Ab significantly increased DCs activation as reflected in the number of CD80+ and CD86+ activated DCs in the TME compared with both untreated mice and DTA-1 mIgG2a NA. remarkably in the case of CD80+ activated DCs, the increase of this population of DCs by DTA-1 mIgG2a Ab compared to DTA-1 mIgG2a NA was even greater than the increase compared to untreated mice. To summarize, DTA-1 mIgG2a, in comparison to an Fc-null antibody, was shown to have enhance anti-tumor respond, enhance Treg reduction, increase in DC percentages in the TME, and elevated DC activation markers, CD86 and CD80.

This Fc-engaging antibody, DTA-1 mIgG2a, was further assessed in various DC knock-out (KO) mice models to determine the dependency of anti-GITR anti-tumor efficacy on the presence of DCs.

### Example 16. In Vivo Anti-Cancer Activity of Mouse aGITR-mIgG2a Variants in a BATF3−/− Mice Model

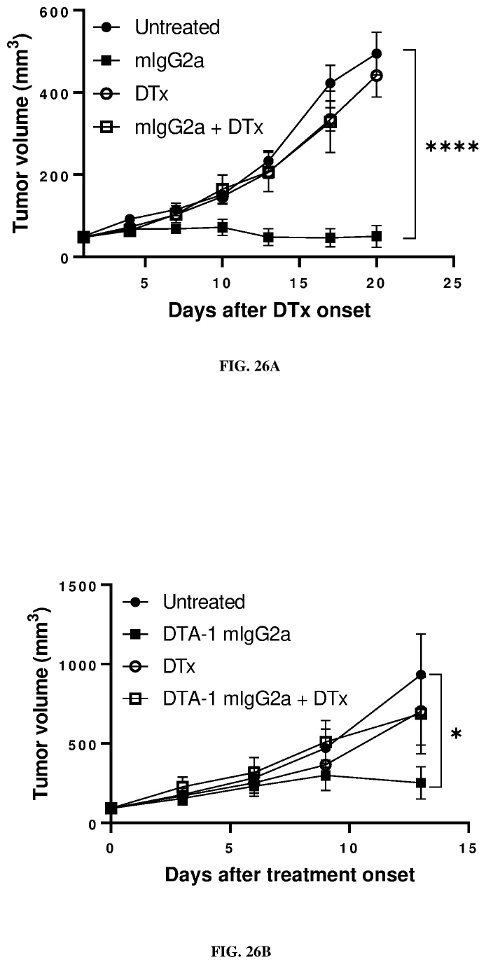

cDC1 cells are a subset of DCs highly effective in presentation of cell-associated antigens in association with class I MHC molecules (MHC-I). This trait of the cDC1 cells contribute to their major role in CD8⁺ T-cell priming, in particular, against infected or tumor cells. BATF3^{-/-} mice, which lack only the DCs subset of cDC1 cells, with MC38 tumors were treated 9 days post inoculation with the DTA-1 mIgG2a Ab or the DTA-1 mIgG2a-NA Ab. Tumor volume (mm³) progression was monitored for 13 days. Untreated mice served as negative control and were sacrificed on day 10 post treatment to avoid unnecessary suffering of the animals. FIG. 25 presents the results of 13 days of tumor volume (mm³) progression in BATF3^{-/-} mice with MC38 tumors treated with 100 μg of DTA-1 mIgG2a (mIgG2a, squares) Ab and 100 μg DTA-1 mIgG2a-NA Ab (mIgG2a NA triangles), or untreated mice (PBS, circles) used as control. Data are represented as mean±SEM, n=8, Unpaired t-test.

FIG. 25 shows that for the first 6 days post treatment tumor progression in all groups is substantially the same. From day 6 to day 9 post treatment, tumor growth rate in mice treated with the Fc-engaging Ab, DTA-1 mIgG2a, starts to decrease, while tumor growth rate in the other groups remains substantially the same. From day 10 post treatment, mice treated with the DTA-1 mIgG2a Ab, showed statistically significant decreases in tumor growth comparing to mIgG2a-N297A and untreated mice. Without wishing to be bound by any theory or mechanism of action, the results indicate that the lack of cDC1 cells hinders the efficacy of Fc-null aGITR Ab, DTA-1 mIgG2a-NA Ab, rendering it ineffective likely due to a deficiency in T cell priming and subsequent lack of agonist effect. The delayed effect of the DTA-1 mIgG2a Ab (detected only from day 10 post treatment) is likely due to its preserved Treg depletion activity but lack of agonism on effector cells.

### Example 17. In Vivo Anti-Cancer Activity of Mouse aGITR-mIgG2a Variants in XCR1-IDTR and ZBTB46-iDTR Mice Models

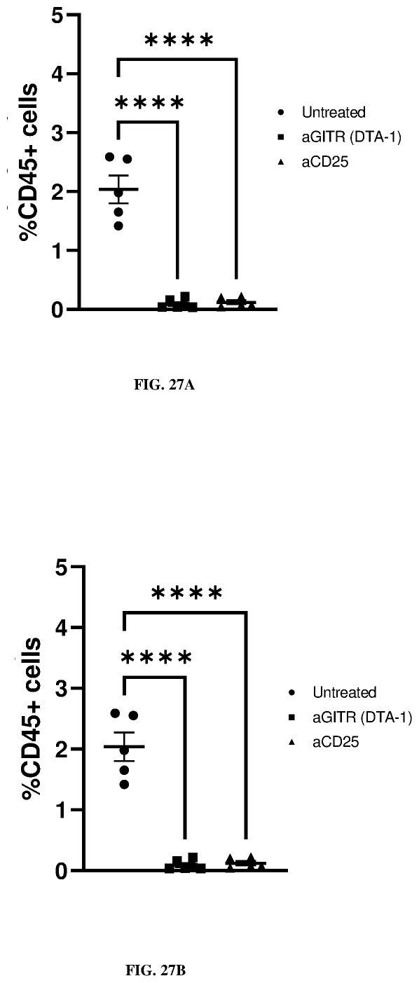

XCR1-iDTR and ZBTB46-iDTR mice models, are models in which DCs can be transiently depleted upon the injection of the Diphtheria Toxin (DT) via the DT receptor (DTR). XCR1-iDTR is an inducible cDC1 cells depleting model and ZBTB46-iDTR is an inducible cDC1 and cDC2 cells depleting model. cDC2 cells are especially efficient in the priming of CD4+ T cells and cDC1 cells, promotes CD8+ T-cells priming, as mentioned before. XCR1-iDTR and ZBTB46-iDTR mice with MC38 tumors, were treated with 100 μg DTA-1 mIgG2a Ab, 20 ng/gr body weight DT, or the combination of 100 μg DTA-1 mIgG2a Ab and DT 20 ng/gr body weight. The DT was injected every other day at 20 ng/g, to deplete DCs. Tumor volume progression was monitored for 13 days (XCR1-iDTR mice) or 20 days (ZBTB46-iDTR mice). FIGS. 26A-26B present the results of tumor volume (mm³) progression in XCR1-iDTR and ZBTB46-iDTR mice treated with 100 μg DTA-1 mIgG2a Ab (mIgG2a, full square), 20 ng/gr body weight DT (DTx, circle), or the combination of 100 μg DTA-1 mIgG2a Ab and 20 ng/gr body weight DT (mIgG2a+DTx, empty square), respectively. Untreated mice were used as control (untreated, circle), for XCR1-iDTR assay n=5-10 mice/group, for ZBTB46-iDTR assay n=8 mice/group. Data are represented as mean±SEM, Unpaired t-test.

As can be seen in FIG. 26A, a similar trend to the BATF3−/− mice model assay is observed in an XCR1-iDTR mouse model, a delayed effect of the DTA-1 mIgG2a Ab on the decrease in tumor growth in mice treated with the DTA-1 mIgG2a Ab compared to mice treated with the combination of DTA-1 mIgG2a Ab and DT. In the XCR1-iDTR assay, the effect of the mIgG2a antibody was abrogated upon DT administration, further suggesting that DCs, specifically cDC1s are necessary for the mediation of successful anti-tumor activity of an Fc-engaging DTA-1 antibody.

As can be seen in FIG. 26B, in the ZBTB46-iDTR model, depleted of both cDC1 and cDC2 cells populations, the efficacy of the Fc-engaging DTA-1 mIgG2a Ab was abolished upon cDC depletion by DT. However, treatment with solely DTA-1 mIgG2a Ab showed lower remarkably and significantly tumor volume progression compared to the treatment with the combination of DTA-1 mIgG2a Ab and DT. Treatment with DTA-1 mIgG2a Ab alone even brought tumor growth to a halt from the early stage of day 3 post treatment.

Without wishing to be bound by any theory or mechanism of action, an Fc-engaging DTA-1 antibody requires engagement with DCs to mediate the enhanced anti-tumor effect observed. In the absence of these cells in the system, it loses its efficacy and possibly even has its effect neutralized to that of an untreated group's effect due to detrimental binding to other effector cells in the environment.

### Example 18. In Vivo aGITR Effect on Tregs Depletion Mediated DC Activation

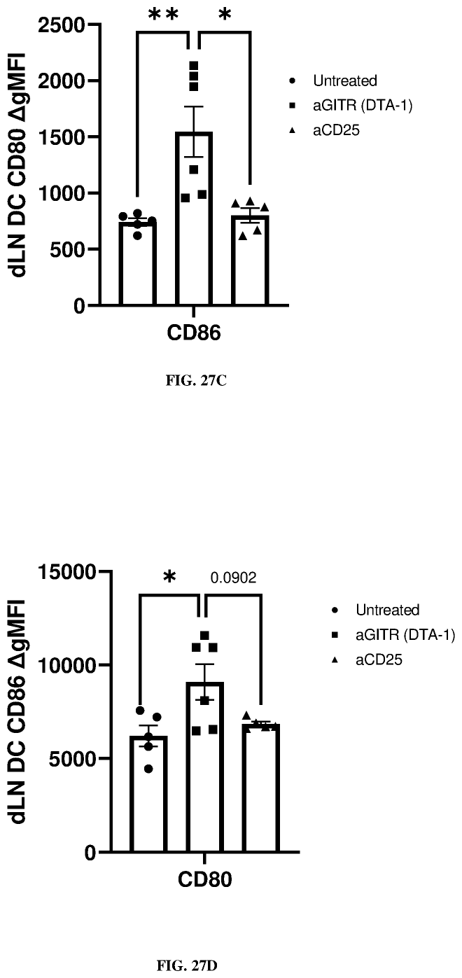

The in vivo effect of anti-GITR Ab on Tregs depletion mediated DC activation was compared to that of anti-CD25, a known in the art, Tregs depleting antibody. To that end, tumors and dLNs from mice C57BL/6J with refractory MC38, treated with 100 μg anti-mouse GITR mIgG2a or 100 μg anti-CD25, were harvested 4 days post treatment. Single cell suspensions were generated from the harvested organs and analyzed for Tregs modulation in the immune compartment in the TME and dLN, or DCs activation in the dLN, by flow cytometry. Untreated mice served as a control. FIGS. 27A-27B present the percentage of Tregs in the TME and percentage of DCs in the dLN, respectively, from mice treated with 100 μg of mouse anti-GITR Ab (aGITR (DTA-1), square), 100 μg of anti-CD25 Ab (aCD25, triangle), or of untreated mice (untreated, circle). Tregs were detected by CD45+CD3+CD11b−CD8−CD4+FoxP3+, and DCs were detected by CD45+CD11b− NK1.1− F4/80− CD11c+ MHCII+. Each dot represents an individual mouse, n=5/6 mice/group. Data are shown as mean±SEM and analyzed using one-way ANOVA. FIGS. 27C-27D present the CD80+ and CD86+ activated DCs, respectively, in dLNs from mice treated with 100 μg of mouse anti-GITR Ab (aGITR (DTA-1), square), 100 μg of mouse anti-CD25 Ab (aCD25, triangle), or of untreated mice (untreated, circle). Activated CD80+ DCs were detected for CD45+ CD11b− NK1.1− F4/80− CD11c+ MHCII+ CD80+, and activated CD86 DCs were detected for CD45+ CD11b− NK1.1− F4/80− CD11c+ MHCII+ CD86+. Each dot represents an individual mouse, n=5/6 mice/group. Delta gMFI was calculated using FLOWJO software. Data are shown as mean±SEM and analyzed using one-way ANOVA.

FIGS. 27A-27B show a significant and almost complete depletion of Tregs in the TME in both anti-GITR AB and anti-CD25 treated mice, compared to the untreated mice. Anti-GITR AB showed a significant increase in the percentage of DCs in the dLN compared to the anti-CD25 and untreated mice. The DCs percentages in untreated mice and anti-CD25 treated mice were similar.

FIGS. 27C-27D show that anti-GITR Ab elicited a significant activation of CD80+ compared to untreated mice and a remarkable increase compared to anti-CD25. Furthermore, anti-GITR Ab elicited a significant activation of CD86+ DCs, compared to anti-CD25 and even higher increase compared to untreated mice.

Since the anti GITR and anti-CD25 treatments mediated an almost complete depletion of Tregs but only anti-GITR exhibited an increase in DCs and elicited their activation, the in vivo Treg depletion mediated DC activation was shown to be GITR dependent.

### Example 19. FcγRIIA and FcγRIIIA Requirement for Optimal Anti mGITR Antibodies-Mediated Anti-Tumor Immunity

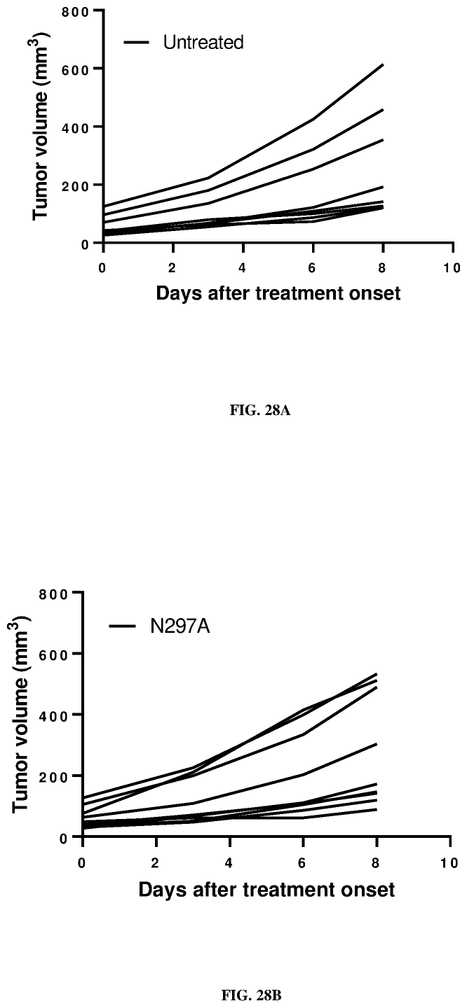

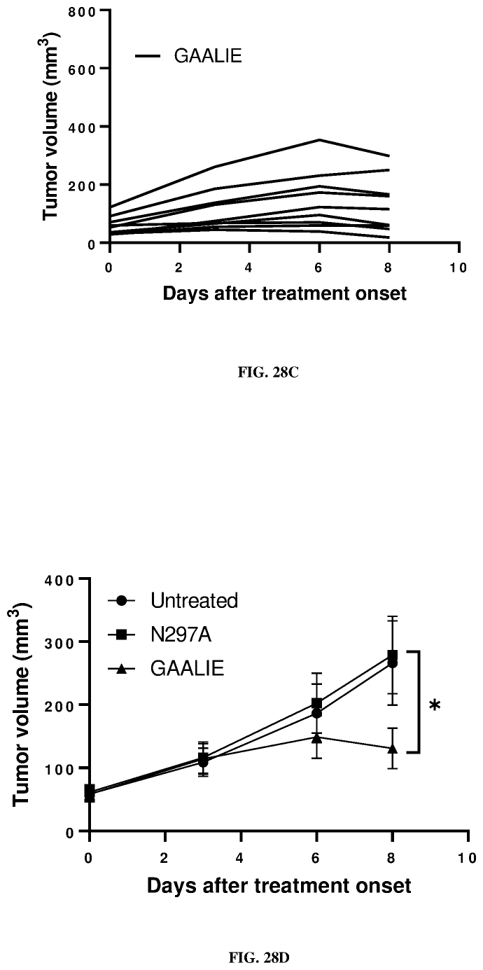

Humanized FcγR mice were inoculated with MC38 cells and once tumor reached average volume of 55 mm³, the mice were treated with chimeric human Fc region variants. The tumor bearing mice were treated every 3 days for a total of 3 treatments (each treatment 100 μg/mice) with IgG1-N297A, or IgG1-GAALIE. Tumor progression was monitored for each animal, as average tumor volume for each treatment group. FIGS. 28A-28C, present tumor volume progression in mice treated with IgG1-N297A (N297A), or with IgG1-GAALIE (GAALIE), untreated mice were used as control (untreated). Each line represents one animal. FIG. 28D presents the results for average tumor volume for each treatment group IgG1-N297A (N297A, n=8), IgG1-GAALIE (GAALIE, n=9), control, untreated mice (untreated n=8). An unpaired two-tailed t test was used to compare between IgG1-N297A and IgG1-GAALIE groups. Data are represented as mean±SEM.

As can be seen from FIGS. 28A-28D, by day 8 post treatment there was apparent tumor volume decrease in mice treated with IgG1-GAALIE, while mice treated with IgG1-N297A exhibit increase in tumor volume. As demonstrated in example 5, GAALIE exhibited FcγRIIa and FcγRIIIa-enhancement which is herein reflects a statistically significant decrease in tumor growth in comparison to IgG1-N297A. The IgG1-GAALIE demonstrates superior anti-tumor activity.

### Example 20. In Vivo Comparison of the Anti-Tumor Immunity of aGITR Variants in MC38 and B16F10 Cancer Inflicted Mice

hFcγR mice inoculated with MC38 or B16F10 cells and are treated with human Fc region variants of DTA-1 (anti-mouse GITR): IgG1-N297A, GAALIE, IgG1, and GA-aFuc. Tumor volume progression and overall survival of the mice are monitored. Next, tumor immune profile post treatment is mapped using single cell multi-omics, which enables the parallel single-cell genomic and transcriptomic simultaneous amplification and a multilevel analysis based on a single cell data.

### Example 21. Generation of Animal Models for the Assessment of the Binding and Activity of Anti-hGITR

Humanized GITR/FcγR, GITR-Tg mice are generated using two different approaches, the genetic engineering approach and the more traditional approach of breeding. For the genetic engineering approach, a human Bacterial Artificial Chromosome clone (BAC) that contains the GITR gene and its associated gene expression genomic regulatory elements is being evaluated. For the breeding approach, the abovementioned humanized FcγR mice are bred with hGITR knock-In mice, in which the mouse GITR was replaced with the human sequence. The anti-hGITR binding and activity is evaluated in vivo using, for example, two commercially available humanized GITR mice, Biocytogen and GenOway.

### Example 22. In Vitro Anti-hGITR Mediated Peripheral Blood Mononuclear Cells (PBMCs) Activation

To test the potential activation of anti-human GITR mAbs, Fresh or frozen human PBMCs from a donor are distributed evenly into tubes and incubated with anti-hGITR Ab and anti human-CD3. PBMCs activation state is evaluated by T cell proliferation (CellTraceViolet dye) and CD25, CD44 expression by flow cytometry. The presence of IL-2 is detected using ELISA.

1-42. (canceled)

43. An isolated afucosylated antibody that specifically binds Glucocorticoid-induced TNFR-related (GITR) protein, wherein the antibody comprises a variable region (Fab), and a modified human IgG1 constant region (Fc), wherein the modified human IgG comprises an Asparagine residue (N) at position 297, the substitution G236A of amino acid residue Glycine (G) at position 236 to Alanine residue (A), (herein afuco-G236A); or,
wherein the modified human IgG comprises the amino acid substitutions of: the Glycine residue at position 236 to an Alanine residue (G236A), substitution of the Alanine residue at position 330 to a Leucine residue (A330L), and substitution of the Isoleucine residue at position 332 to a Glutamic acid residue (I332E), (herein GAALIE).

44. The antibody according to claim 43, wherein the GITR is human GITR (hGITR).

45. The antibody according to claim 44, wherein the Fab of the antibody comprises a set of 6 CDR sequences, wherein the set is selected from the group consisting of:
i. Heavy chain (HC) CDR1 comprising the sequence GFSLSTSGMG (SEQ ID NO: 1), HC CDR2 comprising the sequence IWWDDDK (SEQ ID NO: 2), HC CDR3 comprising the sequence ARTRRYFPFAY (SEQ ID NO: 3), light chain (LC) CDR1 comprising the sequence QNVGTN (SEQ ID NO: 4), LC CDR2 comprising the sequence SAS or the sequence SAST (SEQ ID NO: 5), LC CDR3 comprising the sequence QQYNTDPLT (SEQ ID NO: 6);
ii. HC CDR1 comprising the sequence SYGMH (SEQ ID NO: 7), HC CDR2 comprising the sequence VIWYEGSNKYYADSVKG (SEQ ID NO: 8), HC CDR3 comprising the sequence GGSMVRGDYYYGMDV (SEQ ID NO: 9), LC CDR1 comprising the sequence RASQGISSALA (SEQ ID NO: 10), LC CDR2 comprising the sequence DASSLES (SEQ ID NO: 11), and LC CDR3 comprising the sequence QQFNSYPYT (SEQ ID NO: 12); and
iii. HC CDR1 comprising the sequence GYTFTRYW (SEQ ID NO: 25), HC CDR2 comprising the sequence IYPGDGDT (SEQ ID NO: 26), HC CDR3 comprising the sequence ARNPLTTATAWFVY (SEQ ID NO: 27), LC CDR1 comprising the sequence ENIYSN (SEQ ID NO: 28), LC CDR2 comprising the sequence AAT, and LC CDR3 comprising the sequence QHFWGPPWT (SEQ ID NO: 29).

46. The afuco-G236A antibody according to claim 43, comprising a heavy chain sequence set forth in SEQ ID NO: 13, a light chain sequence set forth in SEQ ID NO: 14, or both.

47. The GAALIE antibody according to claim 43 comprising a heavy chain sequence set forth in SEQ ID NO: 15, a light chain sequence set forth in SEQ ID NO: 14, or both.

48. The antibody according to claim 43 wherein the antibody is a chimeric antibody or a humanized antibody.

49. A conjugate comprising an antibody according to claim 43.

50. A polynucleotide sequence encoding at least one chain of an antibody of claim 43.

51. The polynucleotide sequence according to claim 50, comprising a sequence selected from SEQ ID NO: 22 and SEQ ID NO: 23, encoding an antibody heavy chain; or a variant thereof having at least 80% sequence identity with said polynucleotide sequence.

52. A vector, plasmid or construct comprising at least one polynucleotide sequence according claim 50, or a host cell comprising said vector, plasmid or construct.

53. The vector, plasmid or construct according to claim 52, comprising a polynucleotide sequence selected from SEQ ID NO: 22 and SEQ ID NO: 23, encoding an antibody heavy chain; or a variant thereof having at least 80% sequence identity; and a polynucleotide sequence of SEQ ID NO: 24 or a variant thereof having at least 80% sequence identity.

54. A composition comprising a plurality of antibodies according to claim 43, wherein the Fc region comprises an Asparagine residue (N) at position 297, the substitution G236A of amino acid residue Glycine (G) at position 236 to Alanine residue (A), and wherein about 65-100% of the antibodies in the composition comprise a mature core carbohydrate structure which lacks fucose, attached to the Asparagine residue (N) at position 297 of the Fc region.

55. The composition according to claim 54, wherein about 80-100% of the antibodies in the composition comprise a mature core carbohydrate structure which lacks fucose, attached to the Asparagine residue (N) at position 297 of the Fc region. A pharmaceutical composition comprising as an active ingredient at least one antibody according to any one of claims 1-7, or an antibody conjugate according to claim 8, or the composition of claim 10, and at least one carrier, excipient, or diluent.

56. A kit comprising an antibody according to claim 43, being packaged in a packaging material and identified in print, in or on said packaging material.

57. A method for producing an anti hGITR antibody according to claim 43, comprising:
(i) transfecting of vectors, plasmids, or constructs comprising polynucleotide sequences encoding heavy and light chain pairs, into cells;
(ii) culturing cells which comprise at least one polynucleotide encoding a heavy or light chain of an antibody according to any one of claims 1-7 under conditions which allow expression of the heavy and/or light chains; and
(iii) recovering said chains from the cell.

58. A method for treating cancer or tumor in a subject in need thereof or for enhancing anti-tumor immunity, comprising administering to the subject a therapeutically effective amount of the antibody of claim 43.

59. A method for delaying, slowing, or preventing tumor growth or recurrence, or reducing formation, growth or spread of metastases, comprising administering to the subject the antibody of claim 43.

60. The method of claim 58, wherein the cancer is a metastatic cancer.

61. The method according to claim 43, wherein treatment of cancer or tumor comprises administering or performing at least one additional anti-cancer therapy.

62. A method of enhancing the immune co-stimulatory activity of hGITR, enhancing production of pro-inflammatory cytokines, depleting or inhibiting regulatory T cells (Tregs), or activating dendritic cells, comprising administering to the subject the antibody of claim 43.
